SEQUENCE LISTING (1 sequence)

SEQ ID NO: 1 - protein, 240 aa, Artificial Sequence
  P128 chimera
MSLDSLKKYNGKLPKHDPSFVQPGNRHYKYQCTWYAYNRRGQLGIPVPLWGDAADWIGGAKGAGYGVGRTPKQGACVIWQRGVQGGSPQYGHVAFVEKVLDGGKKIFISEHNYATPNGYGTRTIDMSSAIGKNAQFIYDKKLETPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

(19) United States
(12) Patent Application Publication
(10) Pub. No.: US 2019/0282673 A1
(43) Pub. Date: Sep. 19, 2019

(54) STAPHTAME ACTIVITY ON BIOFILMS

(71) Applicant: GangaGen, Inc., Newark, CA (US)

(72) Inventors: Shankaramurthy Channabasappa, Bangalore (IN); Sandhya Nair, Bangalore (IN); Umender Sharma, Malleshwaram (IN); Bharathi Sriam, Yelahanka New Town, Bangalore (IN); Aradhana Vipra, Bangalore (IN)

(21) Appl. No.: 16/328,226

(22) PCT Filed: Aug. 25, 2017
(86) PCT No.: PCT/US2017/048682

§ 371 Date: Feb. 25, 2019

(30) Foreign Application Priority Data
Aug. 26, 2016 (IN) 201641029197
Nov. 10, 2016 (IN) 201641038513
Apr. 14, 2017 (IN) 201741013414

(51) Int. Cl. A61K 38/47; A61K 45/06; A61P 31/04

(52) U.S. Cl. CPC A61K 38/47; A61P 31/04; A61K 45/06

(57) ABSTRACT

Methods and compositions for the treatment of biofilms, particularly comprising Staphylococcal strains, of particular use in treating MRSA infections, including dormant or difficult to treat biofilm forms.

### CROSS-REFERENCES TO RELATED APPLICATIONS

This Patent Cooperation Treaty application claims the benefit of priority to Indian Provisional Patent Application No. 201641029197 filed Aug. 26, 2016, and titled “Staphtame Activity on Biofilms;” to Indian Provisional Patent Application No. 201641038513, filed Nov. 10, 2016, and titled “Staphtame Activity on Biofilms”; and to Indian Provisional Patent Application No. 201741013414, filed Apr. 14, 2017, and titled “Staphtame Activity on Biofilms”; the entire disclosures of which are hereby incorporated by reference herein for all purposes.

### FIELD OF THE INVENTION

The present disclosure relates to the field of biotechnology, particularly regarding therapy and treatment of bacterial infections. Compositions and methods useful for treatment of various bacterial infections are described.

### BACKGROUND OF THE INVENTION

Bacteria are ubiquitous, and are found in virtually all habitable environments. They are common and diverse ecologically, and find unusual and common niches for survival. They are present throughout the environment, and are present in soil, dust, water, and on virtually all surfaces. Many are normal and beneficial strains, which provide a synergistic relationship with hosts. Others are not so beneficial, or cause problems along with benefits.

Pathogenic bacteria can cause infectious diseases in humans, in other animals, and also in plants. Some bacteria can only make particular hosts ill; others cause trouble in a number of hosts, depending on the host specificity of the bacteria. Diseases caused by bacteria are almost as diverse as the bacteria themselves and include food poisoning, tooth decay, anthrax, general infectious diseases, and even certain forms of cancer. These are typically the subject of the field of clinical microbiology.

Staphylococcus aureus (S. aureus) is known to form biofilms and the bacteria residing in the biofilms have been shown to be highly resistant to the action of antibiotics. Otto (2008) “Staphylococcal biofilms” Curr. Top. Microbiol. Immunol. 322:207-28. Thus, in clinical conditions where biofilms play a role in pathogenesis, including wounds in diabetic patients and in endocarditis, treatment failures are frequent despite the long duration of many treatments. Thwaites, et al. (2011). UK Clinical Infection Research Group “Clinical management of Staphylococcus aureus bacteremia” Lancet Infect. Dis. 11:208-22. The phenotypic resistance of bacteria residing in biofilms has been attributed to multiple factors including both the biofilm matrix acting as a permeability barrier and the presence of slow growing bacteria with poor metabolic rates known as persisters. Keren, et al. (2004) “Persister cells and tolerance to antimicrobials” FEMS Microbiol. Lett. 230:13-18; Lewis (2008) “Multidrug tolerance of biofilms and persister cells” Curr. Top. Microbiol. Immunol. 322:107-31; and Nguyen, et al. (2011) “Active starvation responses mediate antibiotic tolerance in biofilms and nutrient-limited bacteria” Science 334:982-986.

### BRIEF SUMMARY OF THE INVENTION

Provided herein are methods, combination therapies, and compositions for improved eradication of bacteria. For example, provided is a method comprising administering a P128 chimera (e.g., SEQ ID NO: 1, or an amino acid sequence which lacks the initial methionine of SEQ ID NO: 1) to a subject, wherein the administering prevents formation of, or destroys, a biofilm comprising Staphylococcus in the subject. In preferred embodiments, the administering prevents formation of the biofilm; the administering destroys the biofilm; which can form, for example, on a catheter, implant, prosthesis, valve, bandage, or foreign body.

In another aspect, the invention provides a method comprising administering to a subject a synergistic therapy or combination of composition comprising: a P128 chimera with an antibiotic selected from oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, and/or azithromycin; wherein the combination prevents formation of, or destroys, a biofilm comprising Staphylococcus in the subject. In preferred embodiments, the antibiotic is: oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, or azithromycin; the combination prevents formation of the biofilm; the combination destroys the biofilm; or the biofilm forms on a catheter, implant, prosthesis, valve, surface, bandage, or foreign body, whether in vitro or in vivo.

Another aspect of the invention provides a method comprising administering a synergistic therapy or combination composition comprising: a P128 chimera; and an antibiotic, e.g., selected from oxacillin, linezolid, and daptomycin; wherein the synergistic combination prevents formation of, or destroys, a biofilm comprising Staphylococcus. In various embodiments, the antibiotic is: oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, or azithromycin; the administering prevents formation of the biofilm; the administering destroys the biofilm; the biofilm forms on a catheter, implant, prosthesis, valve, surface, bandage, or foreign body; or the biofilm is in vitro or in vivo.

The invention further provides a method comprising administering a synergistic therapy or combination composition comprising: a P128 chimera; and an antibiotic, e.g., selected from oxacillin, vancomycin, linezolid, daptomycin, gentamycin, ciprofloxacin, cefazolin, clindamycin, rifampicin, tigecycline, dalbavancin, telavancin, ceftobiprole, co-trimethaxazole, or azithromycin; wherein the synergistic combination reduces growth of planktonic Staphylococcus cells. In certain embodiments, the antibiotic is: ciprofloxacin; linezolid; daptomycin; vancomycin; gentamycin, cefazolin, clindamycin, rifampicin, tigecycline, dalbavancin, telavancin, ceftobiprole, co-trimethaxazole, or azithromycin; the reduction in growth: is at least about 10-40%; is at least about 40-80% or is at least about 80-99% or more; the reduction in growth reduces the cells over a period of the administering; or the cells are in vitro or in vivo. The methods, therapy, or combination compositions may also reduce the population or growth of small colony variants of a target infection.

### BRIEF DESCRIPTION OF THE DRAWINGS

FIG. 1 shows scanning electron micrographs of S. aureus BK1 biofilms on the surface of microtitre plates treated with P128 or gentamycin.

FIG. 2 shows biofilm eradication/biomass removal activity of P128: Crystal violet staining, A: Crystal violet staining of S. aureus MW2 biofilms in microtitre plates treated with daptomycin, vancomycin, linezolid or P128 at the indicated concentrations for 2, 4 and 24 h, B: OD 570 readings of P128 and antibiotic treated wells.

FIG. 3 shows anti-biofilm activity of P128 on preformed MRSA biofilms on catheters. A. Safranin stained images of S. aureus MW2 biofilms on the surface of catheters treated with P128 B. Scanning electron micrographs of S. aureus MW2 biofilms on the surface of catheters treated with P128 or vancomycin.

FIG. 4A shows S. aureus ATCC29213 biofilms in microtitre plates were treated with the indicated concentrations of P128 for 6 h and the cell viability was determined by plating on TSB agar plates. FIG. 4B shows S. aureus MW2 biofilm on catheter surface-Viable cells remaining on the catheter surface after treatment with P128 or antibiotics. Vanco: Vancomycin, Dapto: Daptomycin

FIG. 5 shows prevention of multi-species biofilms by P128 by virtue of its ability to kill S. aureus. The first three tubes contain Pipette tips transferred from cultures treated with increasing concentrations of P128 (10, 50 and 250 μg/mL), while the last tube contains a tip from untreated culture. The biofilm formation could only be seen in the last tube.

FIGS. 6A-C show activity of P128 and SOC antibiotics on S. epidermidis (FIG. 6A) S. haemolyticus (FIG. 6B) and S. lugdunensis (FIG. 6C) biofilm-P128 at 8 μg/mL (1×MIC) removed all visual biomass indicating activity on preformed biofilm, whereas the SOC antibiotics at 250×MIC or 100×MIC failed to remove biomass.

FIG. 7 shows anti-biofilm activity of P128 on preformed MRSE biofilms on catheters—Scanning electron micrographs of S. epidermidis B9470 biofilms on the surface of catheters treated with P128 or vancomycin.

FIG. 8 shows eradication of 48 h preformed CoNS biofilms on the surface of catheters by P128 visualized by scanning electron microscopy.

FIG. 9A shows that the hemB mutant showed large colonies only around the disc loaded with Haemin. FIG. 9B shows that the menD mutant showed large colonies only around the disc loaded with Menadione. FIG. 9C shows that DMSO did not affect the growth of bacteria tested (Assay control). FIG. 9D shows P128 activity by lawn inhibition assay. Arrow indicates zone of inhibition.

FIG. 10 shows time kill curves in P128 in serum.

FIG. 11 shows the effect of P128 on S. aureus biofilm formed on implanted catheter surface, visualized by safranin-stain.

FIG. 12 shows synergy of P128 with Vancomycin: Efficacy of P128 on biofilm formed on catheters implanted subcutaneously in mice, visualized by safranin-staining.

### DETAILED DESCRIPTION OF THE INVENTION

### I. Introduction

Staphylococcus aureus is responsible for causing a variety of community acquired and hospital acquired infections in humans all over the world. See Nizet and Bradley “Staphylococcal infections” pp 489-515 in Remington, et al. (eds., 2011) Infectious Diseases of the Fetus and Newborn Infant (7th Ed.) Elsevier, Philadelphia. A significant number of the clinical isolates of S. aureus have evolved to become resistant to commonly used antibiotics. Emergence of hospital and community associated methicillin resistant S. aureus (MRSA) has worsened the situation further. Yayan, et al. (2015) “No Outbreak of Vancomycin and Linezolid Resistance in Staphylococcal Pneumonia over a 10-Year Period” PLoS One 10:e0138895. Resistance has also been reported against both recently-introduced and last-resort drugs used for treating S. aureus such as vancomycin, daptomycin and linezolid. Kelley, et al. (2011) “Daptomycin non-susceptibility in vancomycin-intermediate Staphylococcus aureus (VISA) and heterogeneous-VISA (hVISA): implications for therapy after vancomycin treatment failure” J. Antimicrob. Chemother. 66:1057-60 and Gu, et al. (2013) “The emerging problem of linezolid-resistant Staphylococcus” J. Antimicrob. Chemother. 68:4-11. Thus, there is an urgent need to develop new therapies against this important human pathogen.

The majority of the conventional drugs have been shown to have poor anti-persister activity. Rogers, et al. (2012) “Enhancing the utility of existing antibiotics by targeting bacterial behaviour?” Br. J. Pharmacol. 165:845-57. Thus, drugs which show potent bactericidal activity on non-replicating or slowly replicating persisters are expected to be more effective in eradicating biofilms. Lewis (2008) “Multidrug tolerance of biofilms and persister cells” Curr. Top. Microbiol. Immunol. 322:107-31. Towards this end, alternate approaches for killing bacteria in biofilms are being investigated. Chung and Toh (2014) “Anti-biofilm agents: recent breakthrough against multi-drug resistant Staphylococcus aureus” Pathog. Dis. 70:231-39. Included among these are bacteriophages and phage derived proteins (enzybiotics), which have been found to kill bacteria in biofilms, thus offering a viable alternative. Parasion, et al. (2014) “Bacteriophages as an alternative strategy for fighting biofilm development” Pol. J. Microbiol. 63:137-45. A S. aureus specific bacteriophage has been shown to be efficacious in an in vivo animal infection model involving biofilms. Seth, et al. (2013) “Bacteriophage therapy for Staphylococcus aureus biofilm-infected wounds: a new approach to chronic wound care” Plast Reconstr. Surg. 131:225-34. Many enzybiotics do not require metabolically active bacteria for inhibitory action and thus can effectively kill non-replicating bacteria. Loeffler, et al. (2001) “Rapid killing of Streptococcus pneumoniae with a bacteriophage cell wall hydrolase” Science 294:2170-2172; and Paul, et al. (2011) “A novel bacteriophage Tail-Associated Muralytic Enzyme (TAME) from Phage K and its development into a potent antistaphylococcal protein” BMC Microbiol. 11:226. In fact, one of the most commonly used assays for measuring bactericidal activity of enzybiotics is the OD fall assay, which measures lysis of bacteria in a buffer solution. Schuch, et al. (2014) “Combination therapy with lysin CF-301 and antibiotic is superior to antibiotic alone for treating methicillin-resistant Staphylococcus aureus-induced murine bacteremia” J. Infect. Dis. 209:1469-78. Lysostaphin, the earliest known cell wall hydrolase, has been shown to eradicate the biofilms formed by either S. aureus or S. epidermidis strains which were resistant to standard of care (SoC) antibiotics oxacillin and vancomycin. Wu, et al. (2003) “Lysostaphin as a potential therapeutic agent for staphylococcal biofilm eradication” Antimicrob. Agents Chemother. 47:3407-14. A number of phage derived lysins have shown potent bactericidal activity on S. aureus biofilms. Pastagia, et al. (2013) “Lysins: the arrival of pathogen-directed anti-infectives” J. Med. Microbiol. 62:1506-16. Despite demonstration of efficacy in vitro and in various animal models, only a few phage lysins have progressed to evaluation in clinical trials. Roach and Donovan (2015) “Antimicrobial bacteriophage-derived proteins and therapeutic applications” Bacteriophage 5:e1062590.

P128 which incorporates a phage tail associated muralytic enzyme (TAME) possessing anti-Staphylococcal activity, is currently under testing in a clinical trial (ClinicalTrials.gov Identifier: NCT01746654) for clearance of S. aureus from the nasal surface of patients including chronic kidney disease patients who carry S. aureus in the nares. P128 has the sequence shown in SEQ ID NO: 1 or, in a typical embodiment, a sequence shown in SEQ ID NO: 1, which lacks the initial methionine). P128 possesses potent anti-staphylococcal activity against sensitive and drug resistant strains of S. aureus growing as planktonic cells or in biofilms. Paul, et al. (2011) “A novel bacteriophage Tail-Associated Muralytic Enzyme (TAME) from Phage K and its development into a potent antistaphylococcal protein” BMC Microbiol. 11:226; Vipra, et al. (2012) “Antistaphylococcal activity of bacteriophage derived chimeric protein P128” BMC Microbiol. 12:41-50; and Drilling, et al. (2016) “Fighting sinus-derived Staphylococcus aureus biofilms in vitro with a bacteriophage-derived muralytic enzyme” Int. Forum Allergy Rhinol. 6:349-55. The mechanism of killing of staphylococci by P128 involves cleavage of the pentaglycine cross bridge of peptidoglycan. Sundarrajan, et al. (2014) “Bacteriophage-derived CHAP domain protein, P128, kills Staphylococcus cells by cleaving interpeptide cross-bridge of peptidoglycan” Microbiology 160:2157-69. Absence of a pentaglycine in species other than staphylococci makes it inactive on other bacteria. P128 is equally active on bacteria growing in media or on bacteria under conditions of non-replication and nutrient starvation. The lack of inhibitory activity on bacteria other than Staphylococci and on eukaryotic cells (Paul, et al. (2011) “A novel bacteriophage Tail-Associated Muralytic Enzyme (TAME) from Phage K and its development into a potent antistaphylococcal protein” BMC Microbiol. 11:226; and George, et al. (2012) “Biochemical characterization and evaluation of cytotoxicity of antistaphylococcal chimeric protein P128” BMC Res. Notes 5:280) predicts it to be a safe drug candidate for treating human infections involving staphylococci. In order to further explore the utility of P128 to treat serious, difficult to treat infections caused by S. aureus such as bacteremia, infective endocarditis, catheter associated infections and chronic diabetic wounds, the anti-staphylococcal activities of P128 in combination with SoC drugs on planktonic cells and biofilms were tested. P128 was found to kill staphylococci in biofilms in a rapid manner and importantly, was highly synergistic with antibiotics in killing S. aureus in biofilms. P128 could also prevent biofilm formation in multi-species model mimicking biofilm formation in chronic wounds. Sun, et al. (2008) “In vitro multispecies Lubbock chronic wound biofilm model” Wound Repair Regen. 16:805-13. Potent anti-biofilm activity of P128 and synergy with SoC antibiotics makes it a good candidate for further development for treating biofilm associated S. aureus infections.

P128 is an anti-staphylococcal protein comprising a cell wall-degrading enzymatic region and a staphylococcus-specific binding region (also called a cell binding domain), which possesses specific and potent bactericidal activity against sensitive and drug resistant strains of S. aureus. To explore P128's ability to kill S. aureus in a range of environments relevant to clinical infection, the anti-S. aureus activity of P128 alone and in combination with vancomycin, ciprofloxacin and gentamycin on both planktonic and biofilm-embedded cells were investigated. In planktonic cells, P128 showed an additive effect in combination with vancomycin and gentamycin, whereas a synergistic effect was seen in combination with ciprofloxacin. P128 was found to have potent anti-biofilm activity on pre-formed S. aureus biofilms as detected by CFU reduction and a colorimetric minimum biofilm inhibitory concentration (MBIC) assay. Scanning electron microscopic images of biofilms formed on the surface of microtitre plates and on catheters showed that P128 could destroy the biofilm structure and lyse the cells. When tested in combination with antibiotics which are known to be poor inhibitors of S. aureus in biofilms, such as gentamycin, vancomycin and ciprofloxacin, P128 showed highly synergistic anti-biofilm activity resulting in much reduced MBIC values of P128 and the individual antibiotics. Additionally, in an in-vitro mixed biofilm model mimicking the wound infection environment. P128 was able to prevent biofilm formation by virtue of its anti-Staphylococcus activity. Potent S. aureus biofilm inhibitory activity of P128, alone and in combination with antibiotics, is an encouraging sign for developing P128 for treating complicated S. aureus infections involving biofilms.

### II. Anti-Bacterial Treatments and Therapies

As described above, the present disclosure is based, in part, upon the recognition that the combination of a biologic with antibacterial chemotherapeutics has synergistic effects on various targets, including biofilms. Described herein is a particular biologic which actssynergistically with at least one or several standard chemotherapeutics, and which can be used to decrease the dose, duration, or number of different chemotherapeutics used for treatment of biofilms.

### III. Definitions

Two or more therapeutic entities exhibit “synergy” when the combinations exhibit a greater effect than the additive effects of the individual entities, e.g., a substantially better effect than would be expected based on the entities' individual activities. For example, drug synergy occurs when two or more drugs can interact in ways that enhance or magnify one or more positive or advantageous effects of those drugs compared to use when not combined together. This is sometimes exploited in combination preparations, where the therapeutics are admixed or combined into a single formulation, which results in administering them together. Alternatively, the individual compositions may be administered separately, e.g., where each is substantially pure, so they are present in the body at the same time. Negative effects of combination are a form of contraindication. e.g., adverse effects from the combinations.

Measures of synergy typically measure the amount of effect of each component alone when compared to a combination. See, e.g., Geary (2013) “Understanding synergy” Am. J Physiol. Endocrin. Metab. 304:E237-E253, DOI: 10.1152/ajpendo.00308.2012; Torella, et al. (2010) “Optimal Drug Synergy in Antimicrobial Treatments” PLoS Comput Biol. 6:e1000796. PMCID: PMC2880566; and Tallarida (2001) “Drug Synergism: Its Detection and Applications” J. Pharmacology and Exptl Therapeutics 298:865-872. Standard measures of synergy include the Fractional Inhibitory Concentration (FIC) index. See Konate, et al. (2012) “Antibacterial activity against β-lactamase producing Methicillin and Ampicillin-resistant Staphylococcus aureus: fractional Inhibitory Concentration Index (FICI) determination” Annals of Clinical Microbiology and Antimicrobials 11:18. FIC can be calculated from the Minimal Inhibitory Concentrations (MIC) of two drugs. X and Y, as follows:

FIC=([X]/MIC_{X})+([Y]/MIC_{Y})

Drug synergy can occur both in biological activity and because of pharmacokinetics, e.g., where one entity significantly affects the pharmacokinetic properties of the other. Shared metabolic enzymes can cause drugs to remain in the bloodstream much longer in higher concentrations than if individually taken, e.g., where both entities compete for a deactivating mechanism. The biological activity synergy is that normally observed in these combinations.

A P128 chimera will be a chimeric protein comprising the muralytic and targeting domains described in U.S. Pat. No. 8,202,516 or 8,748,150, each of which is incorporated herein by reference. The group of chimeras which make up the group may include variants, e.g., which maintain the functions of the StaphTAME constructs described therein. The TAME designation refers to Tail Associated Muralytic Enzyme, referring to the lytic activity located on phages, typically on the tail of tailed phage, which allows the phage to enter a target host cell. These include variant polypeptides with particular homology ranges to the muralytic domain described. Other members may include chimeras which have similar muralytic functions, or may have different targeting functions which target to the same or similar structural or functional components of targets. In certain embodiments, the targeting domains may be specialized to target biofilm related structures. A preferred embodiment will be representative specific StaphTAME sequences described in the US patents.

Target biofilms of the combinations described herein will be those susceptible to the combinations, preferably those which exhibit synergistic sensitivity. The target biofilms, in most embodiments, will include bacterial components which are susceptible to the P128 chimera when presented in culture distinct from a biofilm format. Thus, the biofilms will generally include one or more susceptible Staphylococcal isolates or strains, or derivatives thereof.

Administering will mean introducing or exposing a target culture or cells to the components of the therapeutic composition. The administering may include multiple components administered together, separately administered simultaneously, or separately administered such that the components are capable to interact because the concentrations are sufficient at a moment in time. Thus, separate administration of components of a combination will often be essentially equivalent to co-administration if the active life times overlap so both are present together.

The combination of components may either prevent formation of a biofilm, or may dissolve or destroy an existing biofilm. Prevention may be useful in different circumstances from destroying preexisting biofilms, and the means to optimally achieve one or the other may involve certain differences.

Biofilms often form on catheters, implants, prosthetic devices, bandages, or foreign bodies introduced into and/or left in the body. The piece may include, e.g., joint replacements, bone substitutes or supplements, lens implants, woven, plastic, ceramic, or metal devices or manufactures, electrical or mechanical devices, etc. The piece may be temporary, intermediate, or permanent. The categories of descriptors are not mutually exclusive, e.g., an artificial heart valve might be considered a valve, an implant, and a foreign body.

The Staphylococcus genus includes many different species, including S. aureus and others. See Nizet and Bradley “Staphylococcal infections” pp 489-515 in Remington, et al. (eds. 2011) Infectious Diseases of the Fetus and Newborn Infant (7th Ed.) Elsevier. Philadelphia. The S. aureus species are coagulase positive, which produce a detectable differentiating enzyme activity. Other species are coagulase negative, but the P128 chimeras generally work on both coagulase positive (e.g., S. aureus species) and coagulase negative (e.g., species other than S. aureus) strains. Importantly, there are both coagulase positive and coagulase negative strains which are methicillin resistant, and the P128 does work on non-S. aureus coagulase-positive targets as well as various other non-S. aureus coagulase-negative targets.

“Coordinated” therapy exists when two or more therapies are used together. The coordinated therapy may be simultaneously applied, or sequentially. Where the pharmacological effect of one remains when the other is provided, they will work together during the period when both are present. In certain embodiments, the different therapies may be administered in succession, which may be specifically ordered or randomly ordered. In some cases, a therapy might incorporate other than a drug, e.g., which might be a procedure such as massage or special breathing methods.

For a therapeutic drug, “administering” is dosing to the subject, and may include many means of administration. Administration can be oral, topical, local, systemic, parenteral, non-parenteral, etc. In many cases, the administering will involve inserting drug into the person, e.g., by injection, inhalation, topical absorption, or other.

Two or more drugs may be provided by “simultaneous” administration, e.g., where both are administered with a short period. The administration of drugs might be co-administered in a single formulation, or each administered in rapid succession. Where administration may involve some period of time, they may be successively administered within one medical procedure or visit. Typically a visit may take up to an hour, or the administration procedure may be an infusion, which may extend for a few hours. In other embodiments, the administrations may be virtually instantaneous, e.g., swallowing of a pill or injection of a small volume.

In some embodiments, the drugs might be provided by “successive” administration, e.g., within reasonably short periods, e.g., hours, or within 2, 3, 5, 7, 10, 14, 17, 21, 24, 18, 30, 34, 38 days, etc. In some embodiments, the drugs are administered close enough in time to retain synergistic effect. In some cases, the drugs may be administered in either order, while in others, one will be indicated to be administered before another. Because the pharmacokinetics of different drugs may differ, the combination may have special temporal windows where both are present at the correct site in appropriate concentrations.

In certain embodiments, the presently disclosed compositions and methods incorporate an additional means to achieve a function of increasing the permeability of a biofilm.

A “chemotherapeutic” is a molecular structure which is a non-protein entity, generally to distinguish from natural or engineered proteins. Chemotherapeutics are typically described as “small molecules,” in contrast to typical protein structures. Thus, antibacterial chemotherapeutics will typically be small molecule drugs, whose molecular sizes are smaller than standard proteins, e.g., smaller than proteins having molecular weights in the 10, 15, 20, 25, or 50 kDa size ranges. Examples of antibacterial chemotherapeutics are antibiotics, such as oxacillin, vancomycin, linezolid, daptomycin, gentamycin, ciprofloxacin, cefazolin, clindamycin, rifampicin, tigecycline, dalbavancin, telavancin, and ceftobiprole. These may be representatives of related classes, defined. e.g., by mechanism of action, structure, or other common features, e.g., oxazolidinones (including linezolid, sutezolid, and AZD5847), BTZ043, and SQ109.

In various embodiments, the present disclosure can be applied to treatment of mammals, reptiles, amphibians, or fish. In particular, among the mammals will be primates (human and non-human), valuable livestock, marine or terrestrial mammals including orcas, dolphins, seals, walruses, tetrapods or bipeds such as zoo and exhibition animals such as elephants, camels, goats, sheep, cows, horses, and species designated or recognized as endangered. Among reptiles include snakes, crocodilians, tortoises, turtles, lizards, and tuataras. Amphibian subjects may include salamanders, frogs, and toads. Fish subjects will often be aquaculture subjects, but may be fish in exhibition aquaria, e.g., where admission is charged to view the fish.

A “combination” package will typically package together a plurality of drugs to be administered to the subject. These may be a combination of pills or therapeutic for administration substantially in a single visit with the subject, whether the subject comes to the health care provider, or the opposite. A plurality of therapeutic agents for the method may be provided in sealed card, sealed container, shrink wrap, or formulated capsules. In some embodiments, the drugs may be orally administered, or may include one or more injectable or inhalable. The health care provider will typically confirm that the subject has been dosed, and often provides some additional incentive to do so, as dosing may result in negative side effects which might appear worse than the bacterial infection.

“Cell wall lytic activity” in a phage context is usually a characterization assigned to a structure based upon testing under artificial conditions, but such characterization can be specific for bacterial species, families, genera, or subclasses (which may be defined by sensitivity). Therefore, a “bacterium susceptible to a cell wall degrading activity” describes a bacterium whose cell wall is degraded, broken down, disintegrated, or that has its cell wall integrity diminished or reduced by a particular cell wall degrading activity or activities. Many other “lytic activities” originate from the host bacterial cells, and are important in cell division or phage release. Other phage derived cell wall degrading activities are found on the phage and have evolved to serve in various penetration steps of phage infection but would be physiologically abortive to phage replication if they kill the host cell before phage DNA is injected into the cell. The structures useful in the penetration steps are relevant in that these activities operate on normal hosts from the exterior. In some embodiments, the cell wall degrading activity is provided by an enzyme that is a non-holin enzyme and/or that is a non-lysin enzyme. In some embodiments, the cell binding activity is provided by an enzyme that is a non-holin enzyme and/or that is a non-lysin enzyme.

An “environment” of a bacterium can include an in vitro or an in vivo environment. In vitro environments are typically found in a reaction vessel, in some embodiments using isolated or purified bacteria, but can include surface sterilization, general treatment of equipment or animal quarters, or public health facilities such as water, septic, or sewer facilities. Other in vitro conditions may simulate mixed species populations, e.g., which include a number of symbiotically or interacting species in close proximity. Much of phage and bacterial study is performed in cultures in which the ratios of target host and phage are artificial and non-physiological. An in vivo environment preferably is in a host organism infected by the bacterium. In vivo environments include organs, such as bladder, kidney, lung, skin, heart and blood vessels, stomach, intestine, liver, brain or spinal cord, sensory organs, such as eyes, ears, nose, tongue, pancreas, spleen, thyroid, etc. In vivo environments include tissues, such as gums, nervous tissue, lymph tissue, glandular tissue, blood, sputum, etc., and may reflect cooperative interactions of different species whose survival may depend upon their interactions together. Catheter, implant, and monitoring or treatment devices which are introduced into the body may be sources of infection under normal usage. In vivo environments also may include the surface of food, e.g., fish, meat, or plant materials. Meats include, e.g., beef, pork, fish, chicken turkey, quail, or other poultry. Plant materials include vegetable, fruits, or juices made from fruits and/or vegetables.

“Introducing” a composition to an environment includes administering a compound or composition, and contacting the bacterium with such. Introducing said compound or composition may often be effected by live bacteria which may produce or release such.

A “cell wall degrading protein” is a protein that has detectable, e.g., substantial, degrading activity on a cell wall or components thereof. “Lytic” activity may be an extreme form or result of the degrading activity. Exemplary bactericidal polypeptides include, e.g., the phage derived ORF56 and P128 chimera construct (e.g., SEQ ID NO: 1, or an embodiment which lacks the initial methionine of SEQ ID NO: 1), structurally related entities, mutant and variants thereof, and other related constructs derived.

Alternative phage derived degrading activities will be identified by their location on the phage tails or target host contact points of natural phage, mutated phase remnants (e.g., pyocins or bacteriocins), or encoded by prophage sequences. Preferred segments are derived, e.g., from bacteriophages, phages of Gram positive and Gram negative bacteria, genome sequence of Staphylococcus species, both coagulase-positive and coagulase-negative strains.

The P128 chimeras of the invention also comprise a staphylococcus-specific binding region which can also be referred to as a “cell binding domain” or “CBD.” This domain is typically a targeting motif, which recognizes the bacterial outer surface. In Gram-positive bacteria, the outer surface of the bacteria is typically the murein layer. Thus, the preferred binding segment for these targets will be cell surface entities, whether protein, lipid, sugar, or combination. Binding segments from known lysozymes, endolysins, and such are known and their properties easily found by PubMed or Entrez searches. Other proteins which bind to bacteria include the PGRPs described below, the TLRs, flagellum and pili binding entities, and phage tail proteins involved in target recognition. In a preferred embodiment, the CBD is fused to a TAME protein or to a cell wall degrading protein, both as disclosed herein. In a further preferred embodiment, the CBD is a heterologous domain as compared to the TAME protein or to cell wall degrading protein. That is, the CBD protein is derived from a non-TAME protein or a non-cell wall degrading protein, or is derived from a cell wall binding protein from a different phage, a bacterium or other organism. Thus, the heterologous CBD domain can be used to direct the TAME protein to specific target bacteria or can be used to increase the target range of the TAME protein.

“Small colony variants” (SCVs) constitute a slow-growing subpopulation of bacteria with distinctive phenotypic and pathogenic traits. See, e.g., Brouillette, et al. (2004) “Persistence of a Staphylococcus aureus small-colony variant under antibiotic pressure in vivo” FEMS Immunology and Medical Microbiology 41:35-41; Proctor, et al. (1998) “The Nature of Problem Bacteria: Is Resistance Enough? Staphylococcal Small Colony Variants Have Novel Mechanisms for Antibiotic Resistance” CID 27(Suppl. 1); Kahl, et al. (2005) “Thymidine-Dependent Staphylococcus aureus Small-Colony Variants Are Associated with Extensive Alterations in Regulator and Virulence Gene Expression Profiles” Infection and Immunity 73:4119-4126. Phenotypically, small colony variants have some or all of: a slow growth rate, atypical colony morphology, and unusual biochemical characteristics: which properties make them a challenge for clinical microbiologists to identify. Clinically, small colony variants are better able to persist in mammalian cells and are less susceptible to antibiotics than their wild-type counterparts, and can cause latent or recurrent infections on emergence. Such problems in vitro translate to problems in situ, e.g., in the infectious context.

The small colony variants often exhibit auxotrophy, which is the inability of an organism to synthesize a particular organic compound required for its growth and metabolism (as defined by IUPAC) as a result of mutational changes, and thus can be dependent upon nutritional supplements provided in the culture medium. Two groups of Auxotrophic mutant SCVs being consistently recovered from clinical specimens: (a) SCVs that are deficient in electron transport: defective in the biosynthesis of menadione or haemin, and this phenotype can be reversed by supplementation with menadione or haemin, as is typical for auxotrophic defects; and (b) SCVs that are deficient in thymidine biosynthesis: thymidine-auxotrophic SCVs have a phenotype that is nearly identical to SCVs with a defect in electron transport, and the basis for this is not understood. A third category has been observed which comprises (c) SCVs for which the auxotrophism cannot be defined: e.g., CO2 is a non-specific stimulant for S. aureus growth. SCVs might also arise from other defects (such as defects in F0F1-ATPase and cytochromes) that would not result in auxotrophy for menadione or haemin yet would result in a deficiency in electron transport.

In contrast to the normal S. aureus phenotype, SCVs typically grow as tiny, non-pigmented, and non-hemolytic colonies, e.g., exhibiting less than about 80%, 60%, 50%. 40%, 30%, 20%, 10%, or less colony size after a selected preferred growth period, or approximate rate of growth in selected conditions. SCVs often (i) produce greatly reduced amounts of α-hemolysin; (ii) persist within host cells in in vitro assays; (iii) are auxotrophic for substrates such as menadione, hemin, thiamine, or thymidine; (iv) exhibit delayed coagulase activity (18-24 h); and (v) can revert to their normal phenotype. For example, thymidine-dependent SCVs display two different colony types. (i) “fried-egg” SCVs with translucent edges surrounding a smaller, elevated pigmented center, and (ii) pinpoint colonies, which are nearly 10 times smaller than the normal S. aureus colony.

SCVs generally differ in their growth rate and/or doubling time; growth phase characteristics from normal strains by extended lag phases (mean difference from the normal S. aureus colony. 2.85 h; range. 1 to 6 h) and lower final densities (mean OD at 578 nm [OD₅₇₈], 4.5; range, 2 to 8 compared to a mean OD₅₇₈ of 12.3; range. 9 to 14 for the normal S. aureus colony. After 48 h of incubation at 37° C. on TSA, hemB SCVs were approximately 1 mm in diameter, whereas colonies of the parent strain were 4 mm or larger in diameter. The doubling times were calculated to be about 22.6±3.3 min for the wild type strain and 53.3±4.8 min for the hemB mutant in MHBCA; SCVs in liquid medium in an overnight culture show the doubling time of normal S. aureus is about 20 min, whereas SCVs double in about 180 min.

“GMP conditions” refers to good manufacturing practices, e.g., as defined by the Food and Drug Administration of the United States Government. Analogous practices and regulations exist in Europe. Japan, and most developed countries.

The term “substantially” in the above definitions of “substantially pure” generally means at least about 60%, at least about 70%, at least about 80%, or more preferably at least about 90%, and still more preferably at least about 95% pure, whether protein, nucleic acid, or other structural or other class of molecules.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma.-carboxyglutamate, and O-phosphoserine. Amino acid analog refers to a compound that has the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain a basic chemical structure as a naturally occurring amino acid. Amino acid mimetic refers to a chemical compound that has a structure that is different from the general chemical structure of an amino acid, but that functions in a manner similar to a naturally occurring amino acid.

“Protein”, “polypeptide”, or “peptide” refers to a polymer in which a substantial fraction or all of the monomers are amino acids and are joined together through amide bonds, alternatively referred to as a polypeptide. When the amino acids are α-amino acids, either the L-optical isomer or the D-optical isomer can be used. Additionally, unnatural amino acids, e.g., β-alanine, phenylglycine, and homoarginine, are also included. Amino acids that are not gene-encoded may also be used in the presently disclosed compositions and methods. Furthermore, amino acids that have been modified to include appropriate structure or reactive groups may also be used. The amino acids can be D- or L-isomer, or mixtures thereof. L-isomers are generally preferred. Other peptidomimetics can also be used. For a general review, see, Spatola, in Weinstein, et al. (eds. 1983) Chemistry and Biochemistry of Amino Acids. Peptides and Proteins. Marcel Dekker, New York, p. 267.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. In particular, fusions of sequence may be generated. e.g., incorporating an upstream secretion cassette upstream of desired sequence to generate secreted protein product.

A “fusion protein” refers to a protein comprising amino acid sequences that are in addition to, in place of, less than, and/or different from the amino acid sequences encoding the original or native full-length protein or subsequences thereof. More than one additional domain can be added to a cell wall lytic protein as described herein, e.g., an epitope tag or purification tag, or multiple epitope tags or purification tags. Additional domains may be attached, e.g., which may add additional outer membrane acting activities (on the target or associated organisms of a mixed colony or biofilm), bacterial capsule degrading activities, targeting functions, or which affect physiological processes, e.g., vascular permeability. Alternatively, domains may be associated to result in physical affinity between different polypeptides to generate multi-chain polymer complexes.

The term “nucleic acid” refers to a deoxyribonucleotide, ribonucleotide, or mixed polymer in single- or double-stranded form, and, unless otherwise limited, encompasses known analogues of natural nucleotides that hybridize to nucleic acids in a manner similar to naturally occurring nucleotides. Unless otherwise indicated or by context, a particular nucleic acid sequence includes the complementary sequence thereof.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes typically include at least promoters and/or transcription termination signals. Typically, the recombinant expression cassette includes a nucleic acid to be transcribed (e.g., a nucleic acid encoding a desired polypeptide), and a promoter. Additional factors necessary or helpful in effecting expression may also be used, e.g., as described herein. In certain embodiments, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette. In certain embodiments, a recombinant expression cassette encoding an amino acid sequence comprising a lytic activity on a cell wall is expressed in a bacterial host cell.

A “heterologous sequence” or a “heterologous nucleic acid”, as used herein, is one that originates from a source foreign to the particular host cell, or, if from the same source, is modified from its original form. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

The term “isolated” refers to material that is substantially or essentially free from components which interfere with the activity of an enzyme or biologic. For a saccharide, protein, or nucleic acid as described herein, the term “isolated” refers to material that is substantially or essentially free from components which normally accompany the material as found in its native state. Typically, an isolated saccharide, protein, or nucleic acid is at least about 80% pure, usually at least about 90%, or at least about 95% pure as measured by band intensity on a silver stained gel or other method for determining purity. Purity or homogeneity can be indicated by a number of means well known in the art. For example, a protein or nucleic acid in a sample can be resolved by polyacrylamide gel electrophoresis, and then the protein or nucleic acid can be visualized by staining. For high resolution of the protein or nucleic, HPLC or a similar means for purification may be utilized.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The terms “identical” or percent “identity,” in the context of two or more nucleic acids (e.g., those that encode SEQ ID NO: 1) or protein sequences (e.g., SEQ ID NO: 1), refer to two or more sequences or subsequences that are the same or have a specified percentage of amino acid residues or nucleotides that are the same, when compared and aligned for maximum correspondence, as measured using one of the sequence comparison algorithms or by visual inspection.

The phrase “substantially identical,” in the context of two nucleic acids or proteins, refers to two or more sequences or subsequences that have, over the appropriate segment, at least greater than about 60% nucleic acid or amino acid sequence identity. 65%, 70%, 75%, 80%, 85%, 90%, preferably 91%, 92%, 93%, 94%, 95%, 96%, 97%, 98% or 99% nucleotide or amino acid residue identity, when compared and aligned for maximum correspondence, as measured using one of the following sequence comparison algorithms or by visual inspection. Preferably, the substantial identity exists over a region of the sequences that corresponds to at least about 13, 15, 17, 23, 27, 31, 35, 40, 50, or more amino acid residues in length, more preferably over a region of at least about 100 residues, and most preferably the sequences are substantially identical over at least about 150 residues. Longer corresponding nucleic acid lengths are intended, though codon redundancy may be considered. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l Acad. Sci. USA 85:2444, by computerized implementations of these and related algorithms (GAP. BESTFIT, FASTA, and TFASTA in the Wisconsin Genetics Software Package, Genetics Computer Group, 575 Science Dr., Madison. Wis.), or by visual inspection (see generally, Current Protocols in Molecular Biology. Ausubel, et al., eds., Current Protocols, a joint venture between Greene Publishing Associates. Inc, and John Wiley & Sons, Inc. (1995 and Supplements) (Ausubel)).

Examples of algorithms that are suitable for determining percent sequence identity and sequence similarity are the BLAST and BLAST 2.0 algorithms, which are described in Altschul, et al. (1990) J. Mol. Biol. 215:403-410 and Altschul, et al. (1977) Nucleic Acids Res. 25:3389-3402, respectively. Software for performing BLAST analyses is publicly available through the National Center for Biotechnology Information (ncbi.nlm.nih.gov/) or similar sources. This algorithm involves first identifying high scoring sequence pairs (HSPs) by identifying short “words” of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10. M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff (1989) Proc. Nat'l Acad. Sci. USA 89:10915).

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or proteins are substantially identical is that the protein encoded by the first nucleic acid is immunologically cross reactive with the protein encoded by the second nucleic acid, as described below. Thus, a protein is typically substantially identical to a second protein, for example, where the two peptides differ only by conservative substitutions. Another indication that two nucleic acid sequences are substantially identical is that the two molecules hybridize to each other under stringent conditions, as described below.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence (e.g., a subsequence of a nucleic acid encoding SEQ ID NO: 1), but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is typically at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C. with wash in 0.2×SSC, and 0.1% SDS at 65° C. For PCR, a temperature of about 36° C. is typical for low stringency amplification, although annealing temperatures may vary between about 32-48° C. depending on primer length. For high stringency PCR amplification, a temperature of about 62° C. is typical, although high stringency annealing temperatures can range from about 50° C. to about 65° C. depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to a protein” or “specifically immunoreactive with”, when referring to an antibody refers to a binding reaction which is determinative of the presence of the protein (e.g., a P128 chimera of the invention) in the presence of a heterogeneous population of proteins and other biologics. Thus, under designated immunoassay conditions, the specified antibodies bind preferentially to a particular protein and do not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies. A Laboratory Manual, Cold Spring Harbor Publications, New York, for a description of immunoassay formats and conditions that can be used to determine specific immunoreactivity.

“Conservatively modified variations” of a particular poly nucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at each position where an arginine is specified by a codon, the codon can be altered to another of the corresponding codons described without altering the encoded protein. Such nucleic acid variations are “silent variations,” which are one species of “conservatively modified variations.” Each polynucleotide sequence described herein which encodes a protein also describes possible silent variations, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid which encodes a protein is typically implicit in each described sequence.

Those of skill recognize that many amino acids can be substituted for one another in a protein without affecting the function of the protein, e.g., a conservative substitution can be the basis of a conservatively modified variant of a protein such as the disclosed cell wall lytic proteins. An incomplete list of conservative amino acid substitutions follows. The following eight groups each contain amino acids that are normally conservative substitutions for one another 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V), Alanine (A); 6) Phenylalanine (F), Tyrosine (Y), Tryptophan (W); 7) Serine (S). Threonine (T), Cysteine (C); and 8) Cysteine (C), Methionine (M) (see, e.g., Creighton (1984) Proteins).

Furthermore, one of skill will recognize that individual substitutions, deletions, or additions which alter, add, or delete a single amino acid or a small percentage of amino acids (typically less than 5%, more typically less than 1%) in an encoded sequence are effectively “conservatively modified variations” where the alterations result in the substitution of an amino acid with a chemically similar amino acid. Conservative substitution tables providing functionally similar amino acids are well known in the art.

One of skill will appreciate that many conservative variations of proteins, e.g., cell wall permeabilizing proteins, and nucleic acids which encode proteins yield essentially identical products. For example, due to the degeneracy of the genetic code, “silent substitutions” (e.g., substitutions of a nucleic acid sequence which do not result in an alteration in an encoded protein) are an implied feature of each nucleic acid sequence which encodes an amino acid. As described herein, sequences are preferably optimized for expression in a particular host cell used to produce the outer membrane acting biologics (e.g., yeast, human, and the like). Similarly, “conservative amino acid substitutions,” in one or a few amino acids in an amino acid sequence are substituted with different amino acids with highly similar properties, are also readily identified as being highly similar to a particular amino acid sequence, or to a particular nucleic acid sequence which encodes an amino acid. Conservatively substituted variations of any particular sequence included in the presently disclosed compositions and methods. See also, Creighton (1984) Proteins, Freeman and Company. In addition, individual substitutions, deletions or additions which alter, add or delete a single amino acid or a small percentage of amino acids in an encoded sequence generally are also “conservatively modified variations”.

The presently disclosed compositions and methods can involve the construction of recombinant nucleic acids and the expression of genes in host cells, e.g., bacterial host cells. Optimized codon usage for a specific host will often be applicable. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology. Ausubel, et al., eds., Current Protocols, a joint venture between Greene Publishing Associates, Inc, and John Wiley & Sons, Inc., (1999 Supplement) (Ausubel). Suitable host cells for expression of the recombinant polypeptides are known to those of skill in the art, and include, for example, prokaryotic cells, such as E. coli, and eukaryotic cells including insect mammalian, and fungal cells (e.g., Aspergillus niger).

Examples of protocols sufficient to direct persons of skill through in vitro amplification methods, including the polymerase chain reaction (PCR), the ligase chain reaction (LCR). Q-betareplicase amplification and other RNA polymerase mediated techniques are found in Berger, Sambrook, and Ausubel, as well as Mullis, et al. (1987) U.S. Pat. No. 4,683,202; PCR Protocols A Guide to Methods and Applications (Innis, et al. eds.) Academic Press Inc. San Diego. Calif. (1990) (Innis); Arnheim and Levinson (Oct. 1, 1990) C&EN 36-47; The Journal Of NIH Research (1991) 3:81-94; (Kwoh, et al. (1989) Proc. Nat'l Acad. Sci. USA 86:1173; Guatelli, et al. (1990) Proc. Nat'l Acad. Sci. USA 87:1874; Lomell, et al. (1989) J. Clin. Chem. 35:1826; Landegren, et al. (1988) Science 241:1077-1080; Van Brunt (1990) Biotechnology 8:291-294; Wu and Wallace (1989) Gene 4:560; and Barringer, et al. (1990) Gene 89:117. Improved methods of cloning in vitro amplified nucleic acids are described in Wallace, et al., U.S. Pat. No. 5,426,039.

### IV. Permeability Acting Biologics

The presently disclosed compositions and methods are based partly upon the recognition that certain permeability boundaries prevent the access of antibacterial chemotherapeutics to reach their proper site of action. In particular, for individual cells, the Gram-positive bacteria peptidoglycan layer is a permeability barrier, with properties which may protect the bacteria from the chemotherapeutic. Similarly, in a biofilm, the combination of different cell types may provide a permeability barrier whereby a chemotherapeutic may be physically or otherwise prevented from accessing otherwise susceptible cells.

### A. Bacterial Peptidoglycan Acting Biologics

Biologics which will act on the known structural components making up the Gram-positive bacteria peptidoglycan will typically cleave bonds cross-linking the peptidoglycan linkages therein. These activities will typically be found in the categories of murein degrading proteins. See U.S. Pat. No. 8,202,516 or 8,748,150. Preferred embodiments of the P128 chimeras are those described, but will include variants of the sequences provided therein. Above some sequence identity, most will preserve the function, and below will retain function at a lower probability. Below some other measure of identity, most will have lesser probability of sharing function, but above will typically have greater. Those with highest identity measures might be expected to have greatest likelihood of similar function. However, diversity from natural sources have been subjected to selection, so the distribution of disparity may be focused on noncritical parts of the protein. By looking carefully at sequence alignments, it may be possible to recognize critical functional motifs, which may lead to more accurate sequence evaluation for sequences likely to retain function.

With a biologic having detectable function, the sensitivity of function to changes can be evaluated. The boundaries of the function may be evaluated by truncation constructs removing segments from the N and C terminus of the sequence. Mutagenesis analyses can evaluate where and how sensitive the function is to conservative or other substitutions. Methods for such are well known in the art, and are described in the references listed herein.

Within each category of function, the structural motifs which are characteristic of a function may be evaluated and identified. Such motifs may be used to screen sequence databases for additional biologics which may exhibit the desired functions.

### B. Permeability Assays

Permeability assays across the bacterial peptidoglycan layer can be based upon outside in or inside out. For example, the assay may be designed to detect when a label reaches the cell surface from the extracellular milieu. Conversely, the cells may normally contain or be loaded with indicator, e.g., in the periplasmic space, and release to the extracellular milieu may be evaluated. Details of the kinetics of indicator passive leakage will need to the determined, and the conditions of assay must be compatible with biologic activity of the tested entity. Often different concentrations of biologic are evaluated. The physiological state of the target strain should be carefully monitored to ensure that linkages targeted by the biologic are present in forms comparable to natural infections.

Assays to monitor how quickly cells can be loaded with indicator, which would grossly reflect permeability of the bacteria peptidoglycan layer, may use a dye or indicator which changes color upon reaching the periplasmic space. The periplasmic space typically has a different pH or oxidation state than outside of the cell, and the kinetics of indicator reaching that location may be monitored over time upon exposure of the cells to the cell wall acting biologic. Biologics having high activity will typically allow more indicator past the barrier than biologics having lower activity. Similarly, larger amounts of entities having a set amount of activity will generally allow more indicator to reach the periplasmic space than lesser amounts.

Conversely, assays may be developed which evaluate the rate of leakage of indicators from the periplasmic space to the external milieu. In some embodiments, the indicator will be a dye which is taken up into the periplasmic space, while in other embodiments, certain entities which normally accumulate in the periplasmic space may traced. Often the target cell may be recombinantly generated to produce a traceable indicator into the periplasmic space. The cell may be loaded up with indicator, then washed so free indicator is removed unless it is intimately associated, e.g., inside the bacteria cell wall. Preferably leakage is slow, unless the permeability barrier is compromised. The ability of test biologics to cause release can be the basis for evaluating activity of the biologics to compromise the Gram-positive bacterial peptidoglycan layer barrier.

Assays may be developed to be performed on plates, which provide a spatial separability. Other assays may be in solution, and may be developed with microfluidic strategies for high throughput evaluation. Fluorescent cell sorting technologies can be easily applied with such formats.

Both assay methods, evaluating permeability from outside to in, or inside to out, can be developed into larger scale assays. These may be developed into more qualitative than quantitative, which may be useful when false positive signals are more problematic than false negatives. With higher throughput assays, testing of ten, hundreds, thousands, or more candidates can be performed simultaneously in parallel. With high throughput, the methodology can be used to evaluate larger scale screening efforts, e.g., of mutagenesis efforts using random mutagenesis, to find entities with the preferred or optimal properties. Moreover, large scale efforts may allow for easier screening of large genetic data sources to test many different alternative sequences expressed in different conditions of growth for expression.

Such screening methods allow for application of the screening on large scales. Gene shuffling strategies can be used to generate products for testing and screening for the desired Gram-positive bacterial peptidoglycan layer permeability.

### V. Biofilms; Phage Derived Functionalities

Biofilms are surface adhered phenotypically heterogeneous communities of microorganisms (Costerton, et al. (1999) “Bacterial biofilms: a common cause of persistent infections” Science 284:1318-22), found both in vitro and in vivo in infected tissues. S. aureus is known to form biofilms in a variety of clinical conditions such as osteomyelitis, indwelling medical device associated infections, endocarditis, chronic wound infection, chronic rhinosinusitis and ocular infections. Archer, et al. (2011) “Staphylococcus aureus biofilms: properties, regulation, and roles in human disease” Virulence 2:445-59. In a chronic wound environment, bacterial contamination leads to colonization of bacteria followed by formation of bacterial biofilms on the surface of dead cells in the wounds. Costerton, et al. (1999) “Bacterial biofilms: a common cause of persistent infections” Science 284:1318-22; Donlan and Costerton (2002) “Biofilms: survival mechanisms of clinically relevant microorganisms” Clin. Microbiol. Rev. 15:167-193; and Parsek and Singh (2003) “Bacterial biofilms: an emerging link to disease pathogenesis” Ann. Rev. Microbiol. 57:677-701. The established biofilms are highly recalcitrant to antibiotics and can evade the immune response. Otto (2008) “Staphylococcal biofilms” Curr. Top. Microbiol. Immunol. 322:207-28; and Lewis (2008) “Multidrug tolerance of biofilms and persister cells” Curr. Top. Microbiol. Immunol. 322:107-31. The biofilms can act as reservoirs of infection and are difficult to eradicate, leading to both treatment failure and recurrent episodes of the disease. It has been proven that one of the major reasons for treatment failure in case of chronic wounds is phenotypic resistance of bacteria present in a biofilm to antimicrobials and to the immune system. Donlan and Costerton (2002) “Biofilms: survival mechanisms of clinically relevant microorganisms” Clin. Microbiol. Rev. 15:167-193. There is physiological heterogeneity amongst cells in biofilms (Stewart (2015) “Antimicrobial Tolerance in Biofilms” Microbiol Spectr. 3:3) and many characteristics of the biofilms contribute to their resistance to antibacterials and immunity, including a protective barrier in the form of the biofilm matrix, expression of specific proteins, low metabolic activity, and induction of a persister state in which bacterial resistance to antimicrobial treatment increases. Lewis (2008) “Multidrug tolerance of biofilms and persister cells” Curr. Top. Microbiol. Immunol. 322:107-31; and Fux, et al. (2005) “Survival strategies of infectious biofilms” Trends Microbiol. 13:34-40. Thus, an ideal anti-biofilm agent should be able to destroy and penetrate the biofilm matrix and should be bactericidal to slowly replicating and persister cell populations within the biofilm.

Bacteriophages and phage derived proteins are emerging as viable alternatives for treating drug resistant infections caused by biofilm forming bacteria. Parasion, et al. (2014) “Bacteriophages as an alternative strategy for fighting biofilm development” Pol. J. Microbiol. 63:137-45 and Pastagia, et al. (2013) “Lysins: the arrival of pathogen-directed anti-infectives” J. Med. Microbiol. 62:1506-16. In this study, the antibacterial properties of P128 on S. aureus biofilms have been examined. P128 showed strong inhibition of S. aureus cells growing in biofilms. P128 was equally efficient in eliminating MSSA and MRSA biofilms from the surface of both microtitre plates and catheters. It has been shown that the constitution of biofilms formed by MSSA and by MRSA are different (McCarthy, et al. (2015) “Methicillin resistance and the biofilm phenotype in Staphylococcus aureus” Front. Cell Infect. Microbiol. 28:5:1), and so P128's ability to act equally well on both is thus an important finding. The ability to eradicate biofilms from the surface of catheters suggests that P128 has the potential to control biofilms in device associated infections caused by S. aureus.

Similar to its rapid activity on planktonic cells, P128 could inhibit the growth of S. aureus in biofilms in a rapid manner, demonstrated by the low MBIC values seen in a 2 h assay using sensitive and resistant strains of S. aureus. The MBIC values were only 1-4 fold higher than the planktonic MICs, demonstrating that P128 has potent activity on S. aureus biofilms. The ability of P128 to destroy the biofilm structure of S. aureus as evidenced by SEM suggests that the biofilm matrix might not be a major barrier for the entry of P128. In addition. P128 can kill cells which are metabolically inactive (e.g., in buffers), and this property could be playing a crucial role in killing poorly metabolizing cells trapped inside biofilms. The anti-biofilm activity of P128 observed in various media, surfaces and strains of S. aureus demonstrates that P128 can eliminate biofilms formed under a variety of physiological conditions.

Because eradication of biofilms by single antimicrobial agents is extremely difficult, discovery of agents showing synergy in inhibiting bacteria in biofilms should lead to better clinical treatment outcomes in S. aureus infections involving biofilms. In addition, combination therapy in serious infections can prevent emergence of drug resistance and can also help in reducing the duration of therapy. Although the combinations of P128 and antibiotics showed modest synergy on planktonic cells of S. aureus, the same combinations showed dramatic effects on biofilms. A dramatic lowering of the MBIC of antibiotics resulting in low FIC index values seen in combinations of P128 with antibiotics, especially with gentamycin, which kills planktonic cells efficiently but had no effect on the biofilms, suggests that P128 can potentiate the effect of antibiotics on biofilms. Because P128 kills bacteria by disrupting the peptidoglycan of the bacterial cell wall, the strong synergy seen with antibiotics may result from an increase in permeability of the cells to the antibiotics.

The ability of P128 to prevent biofilm formation in a mixed culture biofilm model by inhibiting growth of S. aureus suggests that S. aureus plays a major role in biofilm formation in this setting. Based on these results, P128 can be used to help in controlling biofilms in chronic wounds which are infected with multiple bacterial species. Burmolle, et al. (2010) “Biofilms in chronic infections—a matter of opportunity—monospecies biofilms in multispecies infections” FEMS Immunol. Med. Microbiol. 59:324-36; and Wolcott, et al. (2013) “The polymicrobial nature of biofilm infection” Clin. Microbiol. Infect. 19:107-12. Strong synergistic killing of biofilm embedded S. aureus including MRSA by P128 in combination with SoC antibiotics, demonstrates that the combination is useful for treating serious S. aureus infections such as chronic wounds, bacteremia, infective endocarditis and device associated infections.

### VI. Commercial Applications

Various applications of the described methods can be immediately recognized. One important application is as antibacterial treatment of articles which may be contaminated in normal use. Locations, equipment, environments, or the like where target bacteria may be public health hazards may be treated using such entities. Locations of interest include public health facilities where the purpose or opportunity exists to deal with target bacteria containing materials. These materials may include waste products, e.g., liquid, solid, or air. Aqueous waste treatment plants may incorporate such to eliminate the target from effluent, whether by treatment with the enzyme entities directly, or by release of cells which produce such. Solid waste sites may introduce such to minimize possibility of target host outbreaks. Conversely, food preparation areas or equipment need to be regularly cleaned, and the presently disclosed compositions and methods can effectively eliminate target bacteria. Medical and other public environments subject to contamination may warrant similar means to minimize growth and spread of target microorganisms. The methods may be used in contexts where sterilization elimination of target bacteria is desired, including air filtration systems for an intensive care unit.

Alternative applications include use in a veterinary or medical context. Means to determine the presence of particular bacteria, or to identify specific targets may utilize the effect of selective agents on the population or culture. Inclusion of bacteriostatic or bactericidal activities to cleaning agents, including washing of animals and pets, may be desired.

The compositions comprising related biologics can be used to treat bacterial infections of, e.g., humans or animals, alone or in combination with bacteria chemotherapeutics. These biologics can be administered alone or in combination with additional chemotherapeutics or can be administered to a subject that has contracted a bacterial infection in the methods described. In some embodiments, biologics are used with antibiotics to treat infections caused by bacteria that replicate slowly as the killing mechanism does not depend so much upon host cell replication. Many antibacterial agents, e.g., antibiotics, are most useful against replicating bacteria. Bacteria that replicate slowly have doubling times of, e.g., about 1-72 hours or more, 1-48 hours, 1-24 hours, 1-12 hours, 1-6 hours, 1-3 hours, or 1-2 hours. Different types may have different susceptibilities to the combinations.

In some embodiments, these biologics are used to treat humans or other animals that are infected with a bacteria species. In some embodiments, the Gram-positive bacterial peptidoglycan layer acting biologics are used, alone or in combination with other antibiotics, to treat humans or other animals that are infected with one or more bacterial species.

### VII. Administration

The route of administration and dosage will vary with the infecting bacteria strain(s), the site and extent of infection (e.g., local or systemic), and the subject being treated. The routes of administration include but are not limited to: oral, aerosol or other device for delivery to the lungs, nasal spray, intravenous (IV), intramuscular, subcutaneous, intraperitoneal, intrathecal, intraocular, vaginal, rectal, topical, lumbar puncture, intrathecal, and direct application to the brain and/or meninges. Excipients which can be used as a vehicle for the delivery of the therapeutic will be apparent to those skilled in the art. For example, the biologic and/or chemotherapeutic could be in lyophilized form and be dissolved just prior to administration by IV injection. The dosage of administration is contemplated to be in the range of about 0.03, 0.1, 0.3, 1, 3, 10, 30, 100, 300, 1000, 3000, 10⁴, 3×10⁴, 10⁵, 3×10⁵, 10⁶, 3×10⁶, 10⁷, 3×10⁷ or more biologic molecules per bacterium in the host infection. Depending upon the size of the biologic, which may itself be tandemly associated, or in multiple subunit form (dimer, trimer, tetramer, pentamer, and the like) or in combination with one or more other entities, e.g., enzymes or fragments of different specificity, the dose may be about 1 million to about 10 trillion/per kg/per day, and preferably about 1 trillion/per kg/per day, and may be from about 10⁶ linkage cleavage units/kg/day to about 10¹³ linkage cleavage units/kg/day.

The chemotherapeutic component of the combination will generally be administered similarly to how it is used when not in combination with the biologic, though preferably in a smaller number of chemotherapeutic entities, at lower dosage, and/or for a shorter period of treatment.

Methods to evaluate bacteria killing capacity of the presently disclosed combinations are similar to methods used to evaluate therapeutic efficacy of standard bacteria therapies. Serial dilutions of bacterial cultures exposed to the compositions can quantify minimum dosages. Alternatively, comparing total bacterial counts with viable colony units can establish how many, or the fraction of bacteria are viable, and how many have been eliminated.

The therapeutic(s) are typically administered until successful elimination of the pathogenic bacteria is achieved, though broad spectrum formulations may be used while specific diagnosis of the infecting strain is being determined. Thus single dosage forms, as well as multiple dosage forms of the presently disclosed compositions are contemplated, as are methods for accomplishing sustained release means for delivery of such single and multi-dosages forms.

With respect to the aerosol administration to the lungs or other mucosal surfaces, the therapeutic composition is incorporated into an aerosol formulation specifically designed for administration. An example of such an aerosol is the Proventil inhaler manufactured by Schering-Plough, the propellant of which contains trichloromonofluoromethane, dichlorodifluoromethane, and oleic acid. Other embodiments include inhalers that are designed for administration to nasal and sinus passages of a subject or patient. The concentrations of the propellant ingredients and emulsifiers are adjusted if necessary based on the specific composition being used in the treatment. The number of peptidoglycan layer acting biologic molecules to be administered per aerosol treatment will typically be in the range of about 10⁶ to 10¹⁷ molecules, and preferably about 10¹².

Typically, the therapy will decrease bacterial replication capacity by at least about 3 fold, and may affect it by about 10, 30, 100, 300, etc., to many orders of magnitude. However, even slowing the rate of bacterial replication without killing may have significant therapeutic or commercial value. Genetic inactivation efficiencies are typically 0.1, 0.2, 0.3, 0.5, 0.8, 1, 1.5, 2.0, 2.5, 3.0, 3.5, 4, 5, 6, 7, 8, or more log units.

### VIII. Formulations

The presently disclosed compositions and methods further include pharmaceutical compositions comprising at least one P128 chimera biologic with the chemotherapeutic(s), provided in a pharmaceutically acceptable excipient. The formulations and pharmaceutical compositions thus include formulations comprising, with or without antibiotic, an isolated biologic specific for the target bacterium, a mixture of two, three, five, ten, or twenty or more biologics that affect the same or typical bacterial host; and a mixture of two, three, five, ten, or twenty or more biologics that affect different bacteria or different strains of the same bacterium, e.g., a cocktail mixture of biologics that collectively increase the permeability of the bacterial cell wall. In this manner, the presently disclosed compositions of can be tailored to the needs of the patient. The compounds or compositions will typically be sterile or near sterile.

The term “therapeutically effective dose” indicates a dose of each component or combination that produces the effect (e.g., bacteriostatic or bactericidal) for which it is administered. The exact dose will depend on the purpose of the treatment, and will be ascertainable by one skilled in the art using known techniques. See, e.g., Ansel, et al. Pharmaceutical Dosage Forms and Drug Delivery; Lieberman (1992) Pharmaceutical Dosage Forms (vols. 1-3). Dekker, ISBN 0824770846, 082476918X, 0824712692, 0824716981; Lloyd (1999) The Art, Science and Technology of Pharmaceutical Compounding; and Pickar and Pickar-Abernethy (2012) Dosage Calculations. Delmar Cengage Learning, ISBN-10: 1439058474, ISBN013: 9781439058473. As is known in the art, adjustments for protein degradation, systemic versus localized delivery, and rate of new protease synthesis, as well as the age, body weight, general health, sex, diet, time of administration, drug interaction, spectrum of bacterial components in the colony, and the severity of the condition may be necessary, and will be ascertainable with some experimentation by those skilled in the art. In particular, relative amounts of the outer membrane acting biologic, other biologic or polypeptide, and chemotherapeutic may be adjusted and tested for optimal combinations. In particular, the combinations may increase the efficacy of various components such that other components may be reduced or eliminated from the combination. Alternatively, the combination may reduce effective treatment time, which allows for termination of the course of therapy after a shorter term.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions with the subject's immune or other systems. Such may include stabilizers, preservatives, salt, or sugar complexes or crystals, and the like.

Exemplary pharmaceutically carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms.

A composition comprising a biologic as described herein can also be lyophilized using means well known in the art, e.g., for subsequent reconstitution and use as disclosed.

Also of interest are formulations for liposomal delivery, and formulations comprising microencapsulated biologics, including sugar crystals. Compositions comprising such excipients are formulated by well-known conventional methods (see, e.g., Remington's Pharmaceutical Sciences. Chapter 43, 14th Ed., Mack Publishing Col, Easton Pa. 18042, USA).

Pharmaceutical compositions can be prepared in various forms, such as granules, tablets, pills, suppositories, capsules (e.g. adapted for oral delivery), microbeads, microspheres, liposomes, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Formulations may incorporate stabilizing agents, wetting and emulsifying agents, salts for varying the osmotic pressure or buffers for securing an adequate pH value.

The pharmaceutical composition can comprise other components in addition to the outer membrane acting biologic. In addition, the pharmaceutical compositions may comprise more than one active ingredient, e.g., two or more, three or more, five or more, or ten or more different biologics, where the different biologics may be specific for the same, different, or accompanying bacteria. For example, the pharmaceutical composition can contain multiple (e.g., at least two or more) defined cell wall acting biologics, wherein at least two of the biologics in the composition have different target bacteria specificity. In this manner, the therapeutic composition can be adapted for treating a mixed infection of different bacteria, or may be a composition selected to be effective against various types of infections found commonly in a particular institutional environment. A select combination may result, e.g., by selecting different groups of Gram-positive peptidoglycan acting entities derived from various sources of differing specificity so as to contain at least one component effective against different or critical bacteria (e.g., strain, species, etc.) suspected of being present in the infection (e.g., in the infected site) or typically accompanying such infection. As noted above, the cell wall acting biologic can be administered in conjunction with other agents or with one or more conventional antibacterial chemotherapeutic, e.g., antibiotic. In some embodiments, it may be desirable to administer the biologic and antibiotic within the same formulation. Alternatively, different therapeutics may be administered in succession.

### IX. Methodology

In some embodiments, the presently disclosed compositions and methods involve well-known methods general clinical microbiology, general methods for handling bacteriophage, and general fundamentals of biotechnology principles and methods. References for such methods are listed below and are herein incorporated by reference for all purposes.

### A. General Clinical Microbiology

General microbiology is the study of the microorganisms. See, e.g., Sonenshein, et al. (eds. 2002) Bacillus subtilis and Its Closest Relatives: From Genes to Cells Amer. Soc. Microbiol., ISBN: 1555812058, Alexander and Strete (2001) Microbiology: A Photographic Atlas for the Laboratory Benjamin/Cummings, ISBN: 0805327320; Cann (2001) Principles of Molecular Virology (Book with CD-ROM; 3d ed.), ISBN: 0121585336; Garrity (ed. 2005) Bergey's Manual of Systematic Bacteriology (2 vol. 2d ed.) Plenum, ISBN: 0387950400; Salyers and Whitt (2001) Bacterial Pathogenesis: A Molecular Approach (2d ed.) Amer. Soc. Microbiol., ISBN: 155581171 X; Tierno (2001) The Secret Life of Germs: Observations and Lessons from a Microbe Hunter Pocket Star, ISBN: 0743421876; Block (ed. 2000) Disinfection, Sterilization, and Preservation (5th ed.) Lippincott Williams & Wilkins Publ., ISBN: 0683307401; Cullimore (2000) Practical Atlas for Bacterial Identification Lewis Pub., ISBN: 1566703921; Madigan, et al. (2000) Brock Biology of Microorganisms (9th ed.) Prentice Hall, ASIN: 0130819220; Maier, et al. (eds. 2000) Environmental Microbiology Academic Pr., ISBN: 0124975704; Tortora, et al. (2000) Microbiology: An Introduction including Microbiology Place™ Website, Student Tutorial CD-ROM, and Bacteria ID CD-ROM (7th ed.), Benjamin/Cummings, ISBN 0805375546; Demain, et al. (eds. 1999) Manual of Industrial Microbiology and Biotechnology (2d ed.) Amer. Soc. Microbiol., ISBN: 1555811280; Flint, et al. (eds. 1999) Principles of Virology: Molecular Biology, Pathogenesis, and Control Amer. Soc. Microbiol., ISBN: 1555811272; Murray, et al. (ed. 1999) Manual of Clinical Microbiology (7th ed.) Amer. Soc. Microbiol., ISBN: 1555811264; Burlage, et al. (eds. 1998) Techniques in Microbial Ecology Oxford Univ. Pr., ISBN: 0195092236; Forbes, et al. (1998) Bailey & Scott's Diagnostic Microbiology (10th ed.) Mosby, ASIN: 0815125356; Schaechter, et al. (ed. 1998) Mechanisms of Microbial Disease (3d ed.) Lippincott, Williams & Wilkins, ISBN: 0683076051; Tomes (1998) The Gospel of Germs: Men, Women, and the Microbe in American Life Harvard Univ. Pr., ISBN: 0674357078; Snyder and Champness (1997) Molecular Genetics of Bacteria Amer. Soc. Microbiol., ISBN: 1555811027; Karlen (1996) MAN AND MICROBES: Disease and Plagues in History and Modern Times Touchstone Books, ISBN: 0684822709; and Bergey (ed. 1994) Bergey's Manual of Determinative Bacteriology (9th ed.) Lippincott, Williams & Wilkins, ISBN: 0683006037.

### B. General Methods for Handling Bacteriophage

General methods for handling bacteriophage are well known, see, e.g., Snustad and Dean (2002) Genetics Experiments with Bacterial Viruses Freeman; O'Brien and Aitken (eds. 2002) Antibody Phage Display: Methods and Protocols Humana; Ring and Blair (eds. 2000) Genetically Engineered Viruses BIOS Sci. Pub.; Adolf (ed. 1995) Methods in Molecular Genetics: Viral Gene Techniques vol. 6, Elsevier; Adolf (ed. 1995) Methods in Molecular Genetics: Viral Gene Techniques vol. 7, Elsevier; and Hoban and Rott (eds. 1988) Molec. Biol. of Bacterial Virus Systems (Current Topics in Microbiology and Immunology No. 136) Springer-Verlag.

### C. General-Fundamentals of Biotechnology, Principles and Methods

General fundamentals of biotechnology, principles and methods are described. e.g., in Alberts, et al. (2002) Molecular Biology of the Cell (4th ed.) Garland ISBN: 0815332181; Lodish, et al. (1999) Molecular Cell Biology (4th ed.) Freeman, ISBN: 071673706X; Janeway, et al. (eds. 2001) Immunobiology (5th ed.) Garland, ISBN: 081533642X; Flint, et al. (eds. 1999) Principles of Virology: Molecular Biology, Pathogenesis, and Control, Am. Soc. Microbiol., ISBN: 1555811272; Nelson, et al. (2000) Lehninger Principles of Biochemistry (3d ed.) Worth, ISBN: 1572599316; Freshney (2000) Culture of Animal Cells: A Manual of Basic Technique (4th ed.) Wiley-Liss; ISBN: 0471348899; Arias and Stewart (2002) Molecular Principles of Animal Development, Oxford University Press, ISBN: 0198792840; Griffiths, et al. (2000) An Introduction to Genetic Analysis (7th ed.) Freeman, ISBN: 071673771X; Kierszenbaum (2001) Histology and Cell Biology, Mosby, ISBN: 0323016391; Weaver (2001) Molecular Biology (2d ed.) McGraw-Hill, ISBN: 0072345179; Barker (1998) At the Bench: A Laboratory Navigator CSH Laboratory, ISBN: 0879695234; Branden and Tooze (1999) Introduction to Protein Structure (2d ed.), Garland Publishing; ISBN: 0815323050; Sambrook and Russell (2001) Molecular Cloning: A Laboratory Manual (3 vol., 3d ed.), CSH Lab. Press, ISBN: 0879695773; Green and Sambrook (2012) Molecular Cloning: A Laboratory Manual (4th ed.) CSH Press, ISBN-10: 1605500569, ISBN-13: 978-1936113422; Ausubel (ed. 2002) Short Protocols in Molecular Biology (5th ed.), Wiley, ISBN-10: 0471250929, ISBN-13: 978-0471250920; Ausubel (ed. 1995) Current Protocols in Molecular Biology, Wiley & Sons, ISBN-10: 047150338X, ISBN-13: 978-0471503385; Ausubel (ed. 1987) Current Protocols in Molecular Biology, Wiley Online Library, ISBN-10: 0471625949, ISBN-13: 978-0471625940; and Scopes (1994) Protein Purification: Principles and Practice (3d ed.) Springer Verlag, ISBN: 0387940723.

### D. Mutagenesis; Site Specific, Random, Shuffling

Based upon the structural and functional descriptions provide herein, homologs and variants may be isolated or generated which may optimize preferred features. Thus, additional catalytic segments of permeability functions may be found by structural homology, or by evaluating entities found in characteristic gene organization motifs. Microbiologic or eukaryotic genes may be identified by gene arrangement characteristic of genes having function, and may be found in particular gene arrangements, and other entities found in the corresponding arrangements can be tested for a Gram-positive bacterial peptidoglycan layer permeabilizing function. These may also serve as the starting points to screen for variants of the structures, e.g., mutagenizing such structures and screening for those which have desired characteristics, e.g., broader substrate specificity. Standard methods of mutagenesis may be used, see, e.g., Johnson-Boaz, et al. (1994) Mol. Microbiol. 13:495-504; U.S. Pat. Nos. 6,506,602, 6,518,065, 6,521,453, 6,579,678, and references cited by or therein.

Binding or targeting segments can be attached (e.g., in a fusion protein) to the presently described biologics. Prevalent or specific target motifs can be screened for binding domains which interact specifically with them. The target can be a highly expressed protein, carbohydrate, or lipid containing structures found on a particular target strains.

The components of the bacterial cell wall may be shared with components of other bacteria cell walls, or possibly with other bacteria or spores. Phage or bacteria sharing structural features are sources to find functions which can degrade such linkages.

A targeting moiety may increase a local concentration of a catalytic fragment, but a linker of appropriate length may also increase the number of cell wall cleavage events locally. Thus, linkers compatible with the target and catalytic motifs or of appropriate length may be useful and increase the permeability enhancing activity leading greater accessibility of the chemotherapeutics, which may contribute to stasis or killing of target bacteria.

### E. Screening

Screening methods can be devised for evaluating mutants or new candidate functional segments. A library of different outer membrane acting biologics could be screened for presence of such gene products. Binding may use crude bacteria cultures, isolated bacteria cell wall components, peptidoglycan preparations, synthetic substrates, or purified reagents to determine the affinity and number of interactions on target cells. Permeability or wall degrading assays may be devised to evaluate integrity of the Gram-positive bacterial peptidoglycan layer of target strains, lawn inhibition assays, viability tests of cultures, activity on cell wall preparations or other substrates, or release of components (e.g., sugars, amino acids, polymers) of the cell wall upon catalytic action.

Linker features may be tested to compare the effects on binding or catalysis of particular linkers, or to compare the various orientations of fragments. Panels of targets may be screened for catalytic fragments which act on a broader or narrower spectrum of target bacteria, and may include other microbes which may share cell wall components, e.g., spores. This may make use of broader panels of related bacteria strains. Strategies may be devised which allow for screening of larger numbers of candidates or variants.

One method to test for a permeabilizing or cell wall degrading activity is to treat source microorganisms with mild detergents to release structurally associated proteins. These proteins are further tested for permeabilizing or wall degrading activity on bacteria cells. The permeability assays may evaluate permeability from outside the cell to in, or inside to out.

### X. Nucleic Acids Encoding Gram-Positive Bacterial Peptidoglycan Layer Acting Biologics

Nucleic acids have been identified that encode the outer membrane or cell wall acting biologics described above, e.g., P128 chimeras and other phage or bacterial LysB-like biologics. Encoded Gram-positive bacterial peptidoglycan layer acting proteins may have outer membrane degrading activity, and those encoding identified Pfam domains are prime candidates, especially those in the listed Pfams. Alternative sources include genomic sequences which possess characteristic features of “lytic” activity containing elements.

Nucleic acids that encode Gram-positive bacterial peptidoglycan layer or cell wall acting biologics are included in the presently disclosed compositions and methods. Methods of obtaining such nucleic acids will be recognized by those of skill in the art. Suitable nucleic acids (e.g., cDNA, genomic, or subsequences (probes)) can be cloned, or amplified by in vitro methods such as the polymerase chain reaction (PCR), the ligase chain reaction (LCR), the transcription-based amplification system (TAS), or the self-sustained sequence replication system (SSR). Besides synthetic methodologies, a wide variety of cloning and in vitro amplification methodologies are well-known to persons of skill. Examples of these techniques and instructions sufficient to direct persons of skill through many cloning exercises are found in Berger and Kimmel. Guide to Molecular Cloning Techniques. Methods in Enzymology 152 Academic Press, Inc., San Diego, Calif. (Berger); Sambrook, et al. (1989) Molecular Cloning-A Laboratory Manual (2nd ed.) Vol. 1-3, Cold Spring Harbor Laboratory, Cold Spring Harbor Press, NY, (Sambrook, et al.); Current Protocols in Molecular Biology, Ausubel, et al., eds., Current Protocols, a joint venture between Greene Publishing Associates, Inc, and John Wiley & Sons, Inc., (1994 Supplement) (Ausubel); Cashion, et al., U.S. Pat. No. 5,017,478; and Carr, European Patent No. 0,246,864.

A DNA that encodes a Gram-positive bacterial peptidoglycan layer or cell wall acting biologic, can be prepared by a suitable method described above, including, e.g., cloning and restriction of appropriate sequences with restriction enzymes. In one preferred embodiment, nucleic acids encoding Gram-positive bacterial peptidoglycan layer permeabilizing polypeptides are isolated by routine cloning methods. A nucleotide sequence of a Gram-positive bacterial peptidoglycan layer or cell wall acting biologic as provided, e.g., P128 chimeras as described can be used to provide probes that specifically hybridize to a gene encoding the polypeptide; or to an mRNA, encoding a Gram-positive bacterial peptidoglycan layer permeabilizing biologic, in a total RNA sample (e.g., in a Southern or Northern blot). Once the target nucleic acid encoding a Gram-positive bacterial peptidoglycan layer or cell wall acting biologic is identified, it can be isolated according to standard methods known to those of skill in the art (see, e.g., Sambrook, et al. (1989) Molecular Cloning: A Laboratory Manual (2d ed., Vols. 1-3) Cold Spring Harbor Laboratory; Berger and Kimmel (1987) Methods in Enzymology. Vol. 152: Guide to Molecular Cloning Techniques, San Diego: Academic Press, Inc.; or Ausubel, et al. (1987) Current Protocols in Molecular Biology, Greene Publishing and Wiley-Interscience, New York). Further, the isolated nucleic acids can be cleaved with restriction enzymes to create nucleic acids encoding the full-length Gram-positive bacterial peptidoglycan layer permeabilizing polypeptide, or subsequences thereof, e.g., containing subsequences encoding at least a subsequence of a catalytic domain of a Gram-positive bacterial peptidoglycan layer permeabilizing polypeptide. These restriction enzyme fragments, encoding a Gram-positive bacterial peptidoglycan layer permeabilizing polypeptide or subsequences thereof, may then be ligated, for example, to produce a nucleic acid encoding a Gram-positive bacterial peptidoglycan layer permeabilizing polypeptide.

Similar methods can be used to generate appropriate cell wall fragments or linkers between fragments. Binding segments with affinity to prevalent surface features on target bacteria can be identified and include those from, e.g., lysostaphin. Linker segments of appropriate lengths and properties can be used to connect binding and catalytic domains. See, e.g., Bae, et al. (2005) “Prediction of protein interdomain linker regions by a hidden Markov model” Bioinformatics 21:2264-2270; and George and Heringa (2003) “An analysis of protein domain linkers: their classification and role in protein folding” Protein Engineering 15:871-879.

A nucleic acid encoding an appropriate biologic (e.g., a P128 chimera, such as SEQ ID NO: 1), or a subsequence thereof, can be characterized by assaying for the expressed product. Assays based on the detection of the physical, chemical, or immunological properties of the expressed polypeptide can be used. For example, one can identify a Gram-positive bacterial peptidoglycan layer or cell wall acting polypeptide by the ability of a polypeptide encoded by the nucleic acid to increase permeability of bacteria, to degrade, or to digest bacteria cells, e.g., as described herein.

Also, a nucleic acid encoding a desired biologic, or a subsequence thereof, can be chemically synthesized. Suitable methods include the phosphotriester method of Narang, et al. (1979) Meth. Enzymol. 68: 90-99; the phosphodiester method of Brown, et al. (1979) Meth. Enzymol. 68:109-151; the diethylphosphoramidite method of Beaucage, et al. (1981) Tetra. Lett. 22:1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill recognizes that while chemical synthesis of DNA is often limited to sequences of about 100 bases, longer sequences may be obtained by the ligation of shorter sequences.

Nucleic acids encoding a desired polypeptide, or subsequences thereof, can be cloned using DNA amplification methods such as polymerase chain reaction (PCR). Thus, for example, the nucleic acid sequence or subsequence is PCR amplified, using a sense primer containing one restriction enzyme site (e.g., NdeI) and an antisense primer containing another restriction enzyme site (e.g., HindIII). This will produce a nucleic acid encoding the desired polypeptide or subsequence and having terminal restriction enzyme sites. This nucleic acid can then be easily ligated into a vector containing a nucleic acid encoding the second molecule and having the appropriate corresponding restriction enzyme sites. Suitable PCR primers can be determined by one of skill in the art using sequence information provided, e.g., in GenBank or other sources. Appropriate restriction enzyme sites can also be added to the nucleic acid encoding the Gram-positive bacterial peptidoglycan layer permeabilizing biologic or polypeptide subsequence thereof by site-directed mutagenesis. The plasmid containing a Gram-positive bacterial peptidoglycan layer permeabilizing biologic-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis, et al. (1987) U.S. Pat. No. 4,683,202; PCR Protocols A Guide to Methods and Applications (Innis, et al., eds.) Academic Press Inc. San Diego, Calif. (1990) (Innis); Arnheim and Levinson (Oct. 1, 1990) C&EN36-47; The Journal Of NIH Research (1991) 3:81-94; (Kwoh, et al. (1989) Proc. Nat'l Acad. Sci. USA 86:1173; Guatelli, et al. (1990) Proc. Nat'l Acad. Sci. USA 87:1874; Lomell, et al. (1989) J. Clin. Chem. 35:1826; Landegren, et al., (1988) Science 241:1077-1080; Van Brunt (1990) Biotechnology 8: 291-294; Wu and Wallace (1989) Gene 4: 560; and Barringer, et al. (1990) Gene 89:117.

Some nucleic acids encoding bacteria peptidoglycan acting biologics can be amplified using PCR primers based on the sequence of the identified polypeptides.

Other physical properties, e.g., of a recombinant Gram-positive bacterial peptidoglycan layer acting biologic expressed from a particular nucleic acid, can be compared to properties of known desired polypeptides to provide another method of identifying suitable sequences or domains, e.g., of the outer membrane acting biologics that are determinants of bacterial specificity, binding specificity, and/or catalytic activity. Alternatively, a putative Gram-positive bacterial peptidoglycan layer acting biologic encoding nucleic acid or recombinant Gram-positive bacterial peptidoglycan layer permeabilizing biologic gene can be mutated, and its role as a permeabilizing biologic, or the role of particular sequences or domains established by detecting a variation in bacteria effect normally enhanced by the unmutated, naturally-occurring, or control Gram-positive bacterial peptidoglycan layer acting biologic. Mutation or modification of the presently disclosed polypeptides can be facilitated by molecular biology techniques to manipulate the nucleic acids encoding the polypeptides, e.g., PCR. Other mutagenesis or gene shuffling techniques can be applied to the functional fragments described herein, including Gram-positive bacterial peptidoglycan layer acting activities, cell wall acting properties, or linker features compatible with chimeric constructs.

Functional domains of newly identified Gram-positive bacterial peptidoglycan layer acting biologics can be identified by using standard methods for mutating or modifying the polypeptides and testing them for activities such as acceptor substrate activity and/or catalytic activity, as described herein. The sequences of functional domains of the various cell wall acting proteins can be used to construct nucleic acids encoding or combining functional domains of one or more cell wall acting polypeptides. These multiple activity polypeptide fusions can then be tested for a desired bactericidal or bacteriostatic activity. Related sequences based on homology to identified “lytic” activities can be identified and screened for activity on appropriate substrates.

In an exemplary approach to cloning nucleic acids encoding Gram-positive bacterial peptidoglycan layer acting polypeptides, the known nucleic acid or amino acid sequences of cloned polypeptides are aligned and compared to determine the amount of sequence identity between them. This information can be used to identify and select polypeptide domains that confer or modulate cell wall acting polypeptide activities, e.g., target bacterial or binding specificity and/or permeabilizing activity based on the amount of sequence identity between the polypeptides of interest. For example, domains having sequence identity between the outer membrane acting polypeptides of interest, and that are associated with a known activity, can be used to construct polypeptides containing that domain and other domains, and having the activity associated with that domain (e.g., bacterial or binding specificity and/or outer membrane permeabilizing activity).

### XI. Expression of Desired Biologics in Host Cells

Antibacterial (or other) biologics can be expressed in a variety of host cells, including E. coli, other bacterial hosts, and yeast. The host cells are preferably microorganisms, such as, e.g., yeast cells, bacterial cells, or filamentous fungal cells. Examples of suitable host cells include, for example, Azotobacter sp. (e.g., A. vinelandii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C, albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H, anomala and H. jadinii), and Brettanomyces (e.g., B. lambicus and B, anomalus). Examples of useful bacteria include, but are not limited to, Escherichia, Enterobacter, Azotobacter, Erwinia, Klebsielia, Bacillus, Pseudomonas, Proteus, and Salmonella.

Once expressed in a host cell, the antibacterial acting biologics can be used to prevent growth of appropriate bacteria, typically in combination with the chemotherapeutics. In some embodiments, a P128 biologic is used to decrease growth of a target bacterium. In some embodiments, the protein is used to decrease growth, or affect Gram-positive bacterial peptidoglycan layer permeability. Fusion constructs combining such fragments can be generated, including fusion proteins comprising a plurality of bacteria membrane or cell wall permeabilizing activities, including both peptidase and esterase catalytic activities, or combining the activity with another segment, e.g., a targeting segment which binds to cell wall structures. Combinations of degrading activities can act synergistically for better bacteriostatic or bactericidal activity by an accompanying chemotherapeutic. A linker can be incorporated to provide additional volume for catalytic sites of high local concentration near the binding target.

Typically, a polynucleotide that encodes the Gram-positive bacterial peptidoglycan layer acting biologics is placed under the control of a promoter that is functional in the desired host cell. An extremely wide variety of promoters is well known, and can be used in expression vectors, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, provided herein are expression cassettes into which the nucleic acids that encode fusion proteins, e.g., combining a catalytic fragment with a binding fragment, are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change, et al. (1977) Nature 198:1056), the tryptophan (trp) promoter system (Goeddel, et al. (1980) Nucleic Acids Res. 8:4057), the tac promoter (DeBoer, et al. (1983) Proc. Nat'l Acad. Sci. USA 80:21-25); and the lambda-derived pL promoter and N-gene ribosome binding site (Shimatake, et al. (1981) Nature 292:128). The particular promoter system is typically not critical; many available promoters that function in prokaryotes can be used. A bacteriophage T7 promoter is used as an example.

For expression of outer membrane acting polypeptides in prokaryotic cells other than E. coli, a promoter that functions in the particular prokaryotic production species is used. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in an expression cassette. An exemplary RBS in E. coli consists of a nucleotide sequence 3-9 nucleotides in length located 3-11 nucleotides upstream of the initiation codon (Shine and Dalgarno (1975) Nature 254:34; Steitz in Goldberger (ed. 1979) Biological regulation and development: Gene expression (vol. 1, p. 349) Plenum Publishing, NY).

For expression of proteins in yeast, convenient promoters include GAL1-10 (Johnson and Davies (1984) Mol. Cell. Biol. 4:1440-1448) ADH2 (Russell, et al. (1983) J. Biol. Chem. 258:2674-2682), PHO5 (EMBO J. (1982) 6:675-680), and MFα (Herskowitz and Oshima (1982) in Strathern, et al. (eds.) The Molecular Biology of the Yeast Saccharomyces Cold Spring Harbor Lab., Cold Spring Harbor, N.Y., pp. 181-209). Another suitable promoter for use in yeast is the ADH2/GAPDH hybrid promoter as described in Cousens, et al. (1987) Gene 61:265-275 (1987). For filamentous fungi such as, for example, strains of the fungi Aspergillus (McKnight, et al., U.S. Pat. No. 4,935,349), examples of useful promoters include those derived from Aspergillus nidulans glycolytic genes, such as the ADH3 promoter (McKnight, et al. (1985) EMBO J. 4:2093-2099) and the tpiA promoter. An example of a suitable terminator is the ADH3 terminator (McKnight, et al.).

Either constitutive or regulated promoters can be used. Regulated promoters can be advantageous because the host cells can be grown to high densities before expression of the fusion proteins is induced. High level expression of heterologous polypeptides slows cell growth in some situations. An inducible promoter is a promoter that directs expression of a gene where the level of expression is alterable by environmental or developmental factors such as, for example, temperature, pH, anaerobic or aerobic conditions, light, transcription factors, and chemicals. Such promoters are referred to herein as “inducible” promoters, which allow one to control the tinting of expression of the desired polypeptide. For E. coli and other bacterial host cells, inducible promoters are known to those of skill in the art. These include, for example, the lac promoter, the bacteriophage lambda pL promoter, the hybrid trp-lac promoter (Amann, et al. (1983) Gene 25:167; de Boer, et al. (1983) Proc. Nat'l Acad. Sci. USA 80:21), and the bacteriophage T7 promoter (Studier, et al. (1986) J. Mol. Biol.; Tabor, et al. (1985) Proc. Nat'l Acad. Sci. USA 82:1074-78). These promoters and their use are discussed in Sambrook, et al., supra.

A construct that includes a polynucleotide of interest (e.g., outer membrane acting biologic) operably linked to gene expression control signals that, when placed in an appropriate host cell, drive expression of the polynucleotide is termed an “expression cassette.” Expression cassettes that encode fusion proteins are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication.

The construction of polynucleotide constructs generally requires the use of vectors able to replicate in bacteria. A plethora of kits are commercially available for the purification of plasmids from bacteria (see, e.g., EasyPrepJ, FlexiPrepJ, both from Pharmacia Biotech; StrataClean, from Stratagene; and, QIAexpress Expression System. Qiagen). The isolated and purified plasmids can then be further manipulated to produce other plasmids, and used to transfect cells. Cloning in Streptomyces or Bacillus is also possible.

Selectable markers are often incorporated into the expression vectors used to express the desired polynucleotides. These genes can encode a gene product, such as a polypeptide, necessary for the survival or growth of transformed host cells grown in a selective culture medium. Host cells not transformed with the vector containing the selection gene will not survive in the culture medium. Typical selection genes encode polypeptides that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook, et al., supra.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel. Guide to Molecular Cloning Techniques Methods in Enzymology, Volume 152. Academic Press, Inc., San Diego, Calif. (Berger); and Current Protocols in Molecular Biology, Ausubel, et al., eds., Current Protocols, a joint venture between Greene Publishing Associates, Inc, and John Wiley & Sons, Inc. (1998 Supplement) (Ausubel).

A variety of common vectors suitable for use as starting materials for constructing the presently disclosed expression vectors are well known in the art. For cloning in bacteria, common vectors include pBR322 derived vectors such as pBLUESCRIPT™, and lambda phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adenovirus, and baculovirus), episomal virus vectors (e.g., bovine papillomavirus), and retroviral vectors (e.g., murine retroviruses).

The methods for introducing the expression vectors into a chosen host cell are typically standard, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Translational coupling can be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et al. (1988) J. Biol. Chem. 263: 16297-16302.

The polypeptides disclosed herein can be expressed intracellularly, or can be secreted from the cell. Intracellular expression often results in high yields. If necessary, the amount of soluble, active fusion polypeptide can be increased by performing refolding procedures (see, e.g., Sambrook, et al., supra; Marston, et al. (1984) Bio/Technology 2:800; Schoner, et al. (1985) Bio/Technology 3:151). In embodiments in which the desired polypeptide are secreted from the cell, either into the periplasm or into the extracellular medium, the DNA sequence is often linked to a cleavable signal peptide sequence. The signal sequence directs translocation of the fusion polypeptide through the cell membrane. An example of a suitable vector for use in E. coli that contains a promoter-signal sequence unit is pTA1529, which has the E. coli phoA promoter and signal sequence (see, e.g., Sambrook, et al., supra; Oka, et al. (1985) Proc. Nat'l Acad. Sci. USA 82:7212; Talmadge, et al. (1980) Proc. Nat'l Acad. Sci. USA 77:3988; Takahara, et al. (1985) J. Biol. Chem. 260:2670). In another embodiment, the fusion polypeptides are fused to a subsequence of protein A or bovine serum albumin (BSA), for example, to facilitate purification, secretion or stability. Affinity methods, e.g., using the target of the binding fragment can be used.

The Gram-positive bacterial peptidoglycan layer permeabilizing biologics described herein can also be further linked to other bacterial polypeptide segments, e.g., targeting fragments or permeability segments. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook, et al., supra). For certain applications, extraneous sequence can be cleaved from the fusion polypeptide after purification. This can be accomplished by any of several methods known in the art, including cleavage by cyanogen bromide, a protease, or by Factor X.sub.a (see, e.g., Sambrook, et al., supra; Itakura, et al. (1977) Science 198:1056; Goeddel, et al. (1979) Proc. Nat'l Acad. Sci. USA 76:106; Nagai, et al. (1984) Nature 309:810; Sung, et al. (1986) Proc. Nat'l Acad. Sci. USA 83:561). Cleavage sites can be engineered into the gene for the fusion polypeptide at the desired point of cleavage.

More than one recombinant polypeptide can be expressed in a single host cell by placing multiple transcriptional cassettes in a single expression vector, or by utilizing different selectable markers for each of the expression vectors which are employed in the cloning strategy.

A suitable system for obtaining recombinant proteins from E. coli which maintains the integrity of their N-termini has been described by Miller, et al. (1989) Biotechnology 7:698-704. In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

### XII. Purification of Desired Polypeptides

The presently disclosed polypeptides (e.g., P128 chimeras) can be expressed as intracellular proteins or as proteins that are secreted from the cell. For example, a crude cellular extract containing the expressed intracellular or secreted polypeptides can be used in the presently disclosed methods.

Alternatively, the polypeptides can be purified according to standard procedures of the art, including ammonium sulfate precipitation, affinity columns, column chromatography, gel electrophoresis and the like (see, generally. Scopes (1982) Protein Purification Springer-Verlag, N.Y.; Deutscher (1990) Methods in Enzymology (vol. 182) Guide to Protein Purification, Academic Press. Inc. NY). Substantially pure compositions of at least about 70, 75, 80, 85, 90% homogeneity are preferred, and about 92, 95, 98 to 99% or more homogeneity are most preferred. The purified polypeptides can also be used, e.g., as immunogens for antibody production, which antibodies can be used in immunoselection purification methods.

To facilitate purification of polypeptides, the nucleic acids that encode them can also include a coding sequence for an epitope or “tag” for which an affinity binding reagent is available, e.g., a purification tag. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the presently disclosed polypeptides, and corresponding detection systems are known to those of skill in the art, and several are commercially available (e.g., FLAG. Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli “Purification of recombinant proteins with metal chelating adsorbents” in Setlow (ed. 1990) Genetic Engineering: Principles and Methods. Plenum Press, NY; commercially available from Qiagen (Santa Clarita, Calif.)). Purification tags also include maltose binding domains and starch binding domains. Purification of maltose binding domain proteins is known to those of skill in the art.

Other haptens that are suitable for use as tags are known to those of skill in the art and are described, for example, in the Handbook of Fluorescent Probes and Research Chemicals (6th ed., Molecular Probes. Inc., Eugene Ore.). For example, dinitrophenol (DNP), digoxigenin, barbiturates (see, e.g., U.S. Pat. No. 5,414,085), and several types of fluorophores are useful as haptens, as are derivatives of these compounds. Kits are commercially available for linking haptens and other moieties to proteins and other molecules. For example, where the hapten includes a thiol, a heterobifunctional linker such as SMCC can be used to attach the tag to lysine residues present on the capture reagent.

One of skill would recognize that certain modifications can be made to the catalytic or functional domains of the polypeptide without diminishing their biological activity. Some modifications can be made to facilitate the cloning, expression, or incorporation of the catalytic domain into a fusion polypeptide. Such modifications are well known to those of skill in the art and include, for example, the addition of codons at either terminus of the polynucleotide that encodes the catalytic domain, e.g., a methionine added at the amino terminus to provide an initiation site, or additional amino acids (e.g., poly His) placed on either terminus to create conveniently located restriction enzyme sites or termination codons or purification sequences.

It must be noted that as used herein and in the appended claims, the singular forms “a”, “and”, and “the” include plural referents unless the context clearly dictates otherwise. Thus, e.g., reference to “a bacteriophage” includes a plurality of such bacteriophage and reference to a “host bacterium” includes reference to one or more host bacteria and equivalents thereof known to those skilled in the art, and so forth.

Publications discussed herein are provided solely for their disclosure prior to the filing date of the present application. All publications, websites, accession numbers, and patent literature cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the present disclosure that certain changes and modifications can be made thereto without departing from the spirit or scope of the appended claims.

### Experimental

### I. Bacterial Strains and Culture Conditions; Standardization of Biofilm Formation Conditions

The strains used in this study are listed in Table 1. S. aureus cultures were routinely grown in Trypticase soy broth (TSB). LB broth or agar at 37° C.

### IIA. Standardization of Biofilm Formation Conditions

Culture conditions were optimized for reproducibly obtaining a robust biofilm of S. aureus ATCC29213 in microtitre plates. For this purpose, biofilms were generated in microtiter plates and the surface-adhered cultures remaining after washing off the planktonic cells were analyzed at the end of 48 and 72 h by MTT dye assay. In S. aureus ATCC29213 biofilms at the end of 48 hours, an average OD₅₇₀ of 0.08 was observed and approx 10⁶ CFU could be recovered from the wells. On further incubation the bacterial counts increased to 10⁸ CFU (OD₅₇₀=2.0) at the end of 72 h. Based on these results, all the cultures were incubated up to 72 h to allow formation of a thick biofilm. The OD₅₇₀ values obtained at the end of 72 h with various Staphylococcus strains used in this study are shown in Table 1. The 72 h grown biofilms of various S. aureus strains contained roughly 10⁸ CFU per well of microtitre plate.

TABLE 1
Strains used in this study

| | | MRSA |
|---|---|---|
| Isolates/Strains | Source | status |
| S. aureus BK1 | PHRI, New Jersey | MRSA |
| S. aureus B9241 | Gulbarga, India | GMRSA |
| S. aureus ATCC 29213 | ATCC | MSSA |
| S. aureus Mu50 | ATCC number: 700699) | MRSA |
| S. aureus MW2(BK31) | PHRI, New Jersey | MRSA |
| Enterococcus faecalis V583 | (ATCC number: 700802) | — |
| Pseudomonas aeruginosa PAO1 | (ATCC number: BAA-47) | — |

### IIB. MIC and Drug Combination Studies by Checkerboard Assays

MIC was determined using a modified Clinical and Laboratory Standards Institute (CLSI) broth microdilution procedure described earlier in Vipra, et al. (2012) “Antistaphylococcal activity of bacteriophage derived chimeric protein P128” BMC Microbiol. 12:41-50; and Clinical and Laboratory Standards Institute (2012) “Methods for Dilution Antimicrobial Susceptibility Tests for Bacteria That Grow Aerobically; Approved Standard” CLSI document M07-A9.9 ed. Wayne, Pa. USA. In order to determine effects of combinations of P128 with antibiotics, combinations of various dilutions of P128 and a second drug were tested for growth inhibition using a microdilution checkerboard technique. See Lu, et al. (2013) “In vitro activity of sodium new houttuyfonate alone and in combination with oxacillin or netilmicin against methicillin-resistant Staphylococcus aureus” PLoS One 8:e68053. Briefly, S. aureus culture at a final cell number of 5×10⁵ CFU/mL was added to wells of 96-well microtiter plates (precoated with 0.5% BSA), containing two-fold dilutions of P128 and the second drug in cation adjusted Mueller Hinton Broth (CAMHB). The plates were incubated at 37° C. for 24 h and the individual MICs and the combination MICs were read. The fractional inhibitory concentration index (FICI) was determined using the following equation: FICI=(MIC of drug A in the combination/MIC of drug A alone)+(MIC of drug B in the combination/MIC of drug B alone). The combination was considered to be synergistic when the FICI was ≤0.5; additive when FICI was 0.5-1.0; indifferent when FICI was 1-4 and antagonistic when FICI≥4. The experiments were performed in triplicate and repeated twice.

For biofilm inhibitory studies, the wells were washed twice with 1×PBS and challenged with various concentrations of P128 or other antibiotic drugs in LB and incubated for 24 h at 37° C. LB supplemented with 50 μg/mL CaCl₂ was used for daptomycin treatment wells. The contents of the well was aspirated out and discarded. The biofilm adhered to the wells was quantified by MTT assay as described above. The MBIC was defined as the minimum concentration of P128 or the drug showing no colour development. For testing if P128 showed synergy with other drugs, combinations of P128 and antibiotics were tested by the checkerboard method described earlier (Lu, et al. (2013) “In vitro activity of sodium new houttuyfonate alone and in combination with oxacillin or netilmicin against methicillin-resistant Staphylococcus aureus” PLoS One 8:e68053). In each experiment, in addition to the combination MBIC, the MBIC of each drug was also determined individually. The fractional MBIC concentrations were determined by MTT dye method as described above. The FICI and synergy was also calculated in a similar manner.

### Results—Synergy of P128 and Antibiotics on Planktonic Cells

In order to find out whether P128 can act in a synergistic manner with commonly used antibiotics, MIC based growth inhibition assays were performed. MIC based synergy was studied by checkerboard method by determining the FIC index using the method of Lu, et at (2013) “In vitro activity of sodium new houttuyfonate alone and in combination with oxacillin or netilmicin against methicillin-resistant Staphylococcus aureus” PLoS One 8:e68053. The synergistic potential of P128 with vancomycin (van), gentamycin (gen) and ciprofloxacin (cip) to kill planktonic S. aureus cells was tested on one sensitive (ATCC29213) and two resistant strains of S. aureus BK1 (MRSA) and B9241 (GMRSA). The MIC of P128 on these strains was found to range from 4-8 μg/mL, while the MIC of vancomycin was 1 μg/mL (Table 2). Similarly the MIC of ciprofloxacin was 0.4 μg/mL on the sensitive strains and 8-32 μg/mL on fluoroquinolone resistant strains. Gentamycin showed a MIC of 0.4 and 62 μg/mL on sensitive and resistant strains respectively. The combination of P128 and vancomycin showed an additive effect as the FIC index for the three strains was found to be between 0.7 and 1. Similarly. P128 and gentamycin used together on three S. aureus strains showed an additive effect independent of whether the strain was resistant to gentamycin (B9241) or not (ATCC29213 and BK1). In contrast to vancomycin and gentamycin, ciprofloxacin in combination with P128 showed a clear synergistic effect, with FIC index ranging from 0.25 to 0.37. The combination MIC of ciprofloxacin on BK1 and B9241 strains dropped to 8 and 0.25 μg/mL as compared to the individual MICs of >16 μg/mL and 8 μg/mL respectively. In summary, when tested on planktonic S. aureus bacteria, vancomycin and gentamycin showed an additive effect in combination with P128 in inhibiting both MSSA and MRSA, while ciprofloxacin showed synergy in combination with P128.

TABLE 2
Synergy of P128 in combination with SoC antibiotics

| | Vancomycin | | | | Ciprofloxacin | | | | Gentamycin | | | |
|---|---|---|---|---|---|---|---|---|---|---|---|---|
| S. | MIC (μg/mL) | | | | MIC (μg/mL) | | | | MIC (μg/mL) | | | |
| aureus | | | P128 + | | | | P128 + | | | | P128 + | |
| strain* | P128 | Van | van | FICI | P128 | cip | cip | FICI | P128 | gen | gen | FICI |
| ATCC | 4 | 1 | 4 + | 1.12 | 4 | 0.4 | ND | ND | 4 | 0.4 | 1.56 + | 1 |
| 29213 | | | 0.09 | | | | | | | | 0.4 | |
| BK1 | 4 | 1 | 4 + | 1.24 | 4 | 32 | 0.25 + | 0.37 | 4 | 0.4 | 1.56 + | 1 |
| | | | 0.18 | | | | 8 | | | | 0.4 | |
| B9241 | 8 | 1 | 4 + | 0.74 | 8 | 8 | 8 + | 0.28 | 8 | 62 | 31.25 + | 1 |
| | | | 0.18 | | | | 0.25 | | | | 2 | |

ND—Not determined
*ATCC 29213—sensitive to vancomycin, ciprofloxacin and gentamycin; BK1—sensitive to vancomycin, gentamycin and resistant to ciprofloxacin; B9241—sensitive to vancomycin, resistant to gentamycin and ciprofloxacin

III. P128 Synergizes with Antibiotics—Additional Data

For biofilm inhibitory: In order to broaden P128 synergy spectrum with commonly used antibiotics, synergy assays were carried out with three additional antibiotics viz. Tigecycline, Co-trimethaxazole, and azithromycin. Method followed for testing synergy is same as described in para 151.

### Results:

TABLE D5
Synergy of P128 in combination with SoC antibiotics
P128 and Tigecycline

| Sl. | | MIC (μg/mL) | | | |
|---|---|---|---|---|---|
| No. | S. aureus strain | P128 | Tigecycline | P128 + Tigecycline | FICI value |
| 1 | BK18 | 4 | 4 | 0.5 + 0.12 | 0.15 |

P128 and Co-trimethaxazole

| | | MIC (μg/mL) | | | |
|---|---|---|---|---|---|
| Sl. | | | | P128 + Co- | |
| No. | S. aureus strain | P128 | Co-trimethaxazole | trimethaxazole | FICI value |
| 1 | BK18 | 2 | 0.4/2 | 0.5 + 0.1/0.6 | 0.4 |

P128 and Azithromycin

| Sl. | | MIC (μg/mL) | | | |
|---|---|---|---|---|---|
| No. | S. aureus strain | P128 | Azithromycin | P128 + Azithromycin | FICI value |
| 1 | BK18 | 2 | 2 | 0.25 + 0.5 | 0.37 |

### IV. Reversal of Drug Resistant Phenotype of Staphylococcus Clinical Strains by Synergistic Action of P128 and Antibiotics

Infections caused by drug resistant strains of S. aureus and coagulase negative staphylococci (CoNS) are leading causes of morbidity and mortality all over the world. To overcome the challenge of drug resistance, various approaches are being followed to either discover new therapeutics with a novel mechanism of action or that potentiate the efficacy of existing drugs.

To determine whether this synergistic effect would extend to drug-resistant strains. P128 was tested in combination with oxacillin, vancomycin linezolid, cephazolin, ciprofloxacin, by checkerboard assays on strains individually resistant to one of these drugs (Table D3).

Method: Checkerboard Assay:

Bacterial cultures at a final cell number of 5×10⁵ CFU/mL was added to wells of 96-well microtiter plates (precoated with 0.5% BSA), containing two-fold dilutions of P128 and the second drug in cation adjusted Mueller Hinton Broth (CAMHB). The plates were incubated at 37° C. for 24 h and the individual MICs and the combination MICs were read. The fractional inhibitory concentration index (FICI) was determined using the following equation: FICI=(MIC of drug A in the combination/MIC of drug A alone)+(MIC of drug B in the combination/MIC of drug B alone). The combination was considered to be synergistic when the FICI was ≤0.5; additive when FICI was 0.5-1.0; indifferent when FICI was 1-4 and antagonistic when FICI≥4.

TABLE D3
P128 and antibiotic synergy on antibiotic resistant strains
P128 and Vancomycin synergy for Vancomycin resistant strains

| | | MIC (μg/mL) | | | |
|---|---|---|---|---|---|
| Sl. No | VRSA strains | P128 | Vancomycin | P128 + Vancomycin | FICI value |
| 1 | VRS 3b | 0.97 | 32 | 0.24 + 0.25 | 0.20 Synergy |
| 2 | VRS 1 | 1.9 | 32 | 0.24 + 0.5 | 0.13 Synergy |
| 3 | VRS 10 | 3.9 | 16 | 0.24 + 1 | 0.12 Synergy |
| 4 | VRS 2 | 1.9 | 32 | 0.48 + 2 | 0.31 Synergy |
| 5 | VRS 3a | 1.9 | 32 | 0.24 + 1 | 0.15 Synergy |
| 6 | VRS 4 | 1.9 | 32 | 0.48 + 2 | 0.31 Synergy |

P128 and Linezolid synergy for linezolid resistant strains

| | | MIC (μg/mL) | | | |
|---|---|---|---|---|---|
| Sl. No | LRSA Strains | P128 | Linezolid | P128 + Linezolid | FICI value |
| 1 | B9456 | 2 | >32 | 0.25 + 2 | 0.15 Synergy |
| 2 | B9457 | 1 | 8 | 0.25 + 2 | 0.5 Synergy |

P128 and Cephazolin synergy for Cephazolin resistant strains

| | | MIC (μg/mL) | | | |
|---|---|---|---|---|---|
| Sl. No. | S. aureus Strains | P128 | Cephazolin | P128 + Cephazolin | FICI value |
| 1 | COL | 2 | >10 | 0.25 + 0.3 | 0.15 Synergy |
| 2 | USA 300 | 4 | >10 | 1 + 0.6 | 0.31 Synergy |
| 3 | MW2 | 2 | 5 | 0.25 + 0.3 | 0.18 Synergy |

P128 and ciprofloxacin synergy for ciprofloxacin resistant strains

| | | MIC (μg/mL) | | | |
|---|---|---|---|---|---|
| Sl. No | S. aureus strain | P128 | Ciprofloxacin | P128 + Ciprofloxacin | FICI value |
| 1 | BK1 | 4 | 32 | 0.25 + 8 | 0.37 Synergy |
| 2 | B9241 | 8 | 8 | 2 + 0.25 | 0.28 Synergy |

P128 and Daptomycin synergy for Daptomycin resistant strains

| | | MIC (μg/mL) | | | |
|---|---|---|---|---|---|
| Sl. No. | S. epidermidis strains | P128 | Daptomycin | P128 + Daptomycin | FICI value |
| 1 | B9471 | 32 | 2 | 1.0 + 0.5 | 0.28 Synergy |
| 2 | B9472 | 16 | 2 | 2.0 + 0.5 | 0.28 Synergy |
| 3 | B9467 | 32 | 2 | 8 + 0.5 | 0.5 Synergy |

P128 and Oxacillin synergy for Methicillin resistant strains

| | | MIC (μg/mL) | | | |
|---|---|---|---|---|---|
| Sl. No. | MRSA strains | P128 | Oxacillin | P128 + Oxacillin | FICI value |
| 1 | COL | 0.9 | >16 | 0.24 + 0.5 | 0.27 Synergy |
| 2 | USA 300 | 0.45 | >16 | 0.025 + 0.5 | 0.05 Synergy |
| 3 | BK22 | 0.45 | >16 | 0.1 + 0.5 | 0.23 Synergy |
| 4 | MW2 | 0.48 | >16 | 0.03 + 0.5 | 0.07 Synergy |

S. epidermidis Strains

| | | | | | |
|---|---|---|---|---|---|
| 5 | B9470 | 8 | >8 | 1.0 + 0.5 | 0.15 Synergy |
| 6 | B9471 | 8 | >8 | 2.0 + 0.5 | 0.28 Synergy |
| 7 | B9472 | 8 | 16 | 1.0 + 0.25 | 0.14 Synergy |
| 8 | B9473 | 16 | 16 | 4.0 + 0.5 | 0.28 Synergy |
| 9 | B9467 | 32 | 8 | 2.0 + 0.25 | 0.09 Synergy |

Taken together, these results suggest that P128 at sub-MIC concentration can potentially lower the MIC of antibiotics on resistant strains to a level where the bacteria show a drug sensitive phenotype (Table D3). In conclusion, combination of P128 and antibiotics can potentially be developed to treat infections caused by drug resistant strains of staphylococci.

V. P128 Inhibits Growth of S. aureus in Established Biofilms

The ability of P128 to inhibit growth of S. aureus in a preformed biofilm was measured by determining minimum biofilm inhibition concentration (MBIC), defined as minimum concentration showing growth inhibition in a MTT based assay. Since P128 kills Staphylococcus cultures rapidly, it was expected to show a rapid effect on biofilms as well. Paul, et al. (2011) “A novel bacteriophage Tail-Associated Muralytic Enzyme (TAME) from Phage K and its development into a potent antistaphylococcal protein” BMC Microbiol. 11:226. Hence, the MBIC values were determined for this protein on various Staphylococci after exposure times ranging from 2 to 24 h.

Results—

As shown in Table 3, P128 showed rapid inhibition of growth in biofilms as the MBIC values obtained in 2 h was comparable to its MIC values on planktonic cells (Table 2). It was observed that P128 inhibited biofilm formation of both MRSA and MSSA with equal efficiency. There was a small increase in MBIC values up to 8 h, however. The MBIC values of P128 increased drastically after incubation periods beyond 8 h. The reasons for the increase in MBIC values upon prolonged incubation are not currently understood. Based on these results it was decided to determine the MBIC values and the synergy of P128 and antibiotics against various, MSSA and MRSA strains after 6 and 24 h exposure.

TABLE 3
MBIC values of P128 on six strains of staphylococci obtained
at various time points

| | MBIC (μg/ml) | | | | |
|---|---|---|---|---|---|
| Isolates | 2 h | 4 h | 6 h | 8 h | 24 h |
| S. aureus ATCC 29213 | 3.9 | 15.6 | 15.6 | 15.6 | 250 |
| S. epidermidis ATCC | — | 7.8 | 7.8 | 7.8 | >1000 |
| 12228 | | | | | |
| S. aureus 8325-4 | 7.8 | 15.6 | 15.6 | 31.25 | 1000 |
| S. aureus BK1 | 15.6 | 31.25 | 31.25 | 31.25 | 1000 |
| S. aureus B9356 | 7.8 | 15.6 | 31.25 | 62 | >1000 |
| S. aureus B9241 | 15.6 | 125 | 125 | 125 | >1000 |

VI. Minimum Biofilm Inhibitory Concentration (MBIC) and Synergy of P128 with Antibiotics

The assay was optimized using the standard S. aureus strain ATCC 29213. An overnight-grown culture of the strain was diluted 1:40 in LB broth 200 μL of diluted culture was aliquoted into microtiter plate wells. Microplates were placed in a shaker-incubator set to 37° C., and 100 rpm for 24 h followed by 48 h incubation under static conditions at 37° C. The contents of one set of four wells were aspirated and discarded. Wells were washed twice with 1×PBS and the presence of biofilm in the wells at the end of this 72-hour period was determined by metabolic dye-reduction assay method using MTT (3-(4, 5-dimethylthiazol-5-diphenyltetrazolium bromide; Himedia). In this assay, live cells reduce the dye leading to color formation which can be read at 570 nm, and the intensity of color can be correlated to number of live cells. 100 μL of PBS was added to the wells along with 10 μL of MTT solution. The plate was incubated for 2 h in the dark. After this, 110 μL of solvent solution solubilizing agent was added and the plate was incubated for 15 minutes at ambient temperature with gentle agitation. The absorbance was read at 570 nm in a microplate reader. Another set of four wells was processed for harvesting the biofilm and determination of CFUs present by plating on solid media. For biofilm inhibitory studies, the wells were washed twice with 1×PBS and challenged with various concentrations of P128 or other antibiotic drugs and incubated for 24 h at 37° C. The contents of the well was aspirated out and discarded. The biofilm adhered to the wells was quantified by MTT assay as described above. The MBIC was defined as the minimum concentration of P128 or the drug showing no colour development. For testing if P128 showed synergy with other drugs, combinations of P128 and antibiotics were tested by the checkerboard method described by Lu, et al. (2013) “In vitro activity of sodium new houttuyfonate alone and in combination with oxacillin or netilmicin against methicillin-resistant Staphylococcus aureus” PLoS One 8:e68053. In each experiment, in addition to the combination MBIC, the MBIC of each drug was also determined individually. The fractional MBIC concentrations were determined by MTT dye method as described above. The FICI and synergy was also calculated in a similar manner.

Results—P128 Shows Strong Synergy with Antibiotics in Inhibiting S. aureus in Biofilms

In order to determine whether P128 could synergize with known anti-Staphylococcus drugs to inhibit S. aureus in biofilms. MBIC values were determined for various antibiotics in combination with P128: gentamycin, vancomycin and ciprofloxacin on three S. aureus strains of different antibiotic sensitivities. ATCC29213 (sensitive strain), BK1 (MRSA, resistant to ciprofloxacin) and B9241 (MRSA, resistant to gentamycin and ciprofloxacin). The assays were performed in a 96 well checkerboard format using various concentrations of P128 and one of the antibiotics.

Amongst the antibiotics tested on biofilms, gentamycin and ciprofloxacin were found to be the least effective (>625-2500 fold increase over planktonic MIC values), while vancomycin showed inhibition especially at 6 h.

As seen in Table 4, the MBIC values of P128 and the antibiotics in combination were much reduced compared to their individual MBIC values on all three strains of S. aureus. Maximum reduction in MBIC in combination was observed in the case of gentamycin (>250 fold), especially with the two gentamycin sensitive strains (ATCC29213 and BK1). In the majority of the cases (except combination of P128 and vancomycin on ATCC29213 and BK1 strains) the FIC indices of P128 combinations with the antibiotics ranged from 0.06 to 0.53, suggesting a strong synergistic mechanism of inhibition. The synergistic effect of P128 could be seen even in drug combinations on strains which were resistant to the particular drugs: P128 and ciprofloxacin, for example, showed synergy on the ciprofloxacin resistant BK1 strain (FIC index 0.43 and 0.16 in 6 h and 24 h assays). Similarly, on S. aureus B9241, which is resistant to ciprofloxacin and gentamycin, a combination of P128 with gentamycin or ciprofloxacin showed synergistic inhibition with FIC index values ranging from 0.07 to 0.39. Interestingly, despite an increase in MBIC value of P128 over 24 h, the combinations of P128 with all three drugs were found to be highly synergistic in inhibiting growth of S. aureus in biofilms even after 24 h (Table 4, last column).

TABLE 4

| | Activity of P128 and antibiotics, individual and in combination on preformed biofilm | | | | | | | | | | |
|---|---|---|---|---|---|---|---|---|---|---|---|
| | | | | | | MBIC of P128 | | | MBIC of P128 | | |
| | | | | | | and antibiotics | | | and antibiotics | | |
| | MBIC of | | MBIC of | | | (μg/ml) | | | (μg/ml) | | |
| | P128 | | Antibiotics | | | in combination | | | in combination | | |
| | (μg/ml)# | | (μg/mL) | | | (6 h) | | | (24 h) | | |
| Strains | 6 h | 24 h | Antibiotic | 6 h | 24 h | P128 | Antibiotic | FICI | P128 | Antibiotic | FICI |
| S. aureus | 6.04 | 200 | Genta | >1000 | >1000 | 0.78 | 3.9 | 0.13 | 12.5 | 3.1 | 0.06 |
| ATCC | | | Vanco | 7.8 | 218.75 | 2.07 | 3.9 | 0.84 | 10.93 | 39.06 | 0.23 |
| 29213 | | | Cipro | >250 | 6.5 | 0.78 | 0.9 | 0.13 | 12.5 | 0.48 | 0.13 |
| S. aureus | 28.95 | 400 | Genta | >1000 | >1000 | 12.5 | 0.45 | 0.43 | 12.5 | 109.35 | 0.14 |
| BK1 | | | Vanco | 31.25 | 250 | 20.83 | 1.9 | 0.77 | 200 | 7.8 | 0.53 |
| | | | Cipro | >250 | >250 | 12.5 | 0.48 | 0.43 | 21.87 | 27.33 | 0.16 |
| S. aureus | 105.15 | >1000 | Genta | >1000 | >1000 | 16.6 | 26.030 | 0.18 | 200 | 109.35 | 0.30 |
| B9241 | | | Vanco | 31.25 | 125 | 16.6 | 15.6 | 0.65 | 25 | 39.06 | 0.33 |
| | | | Cipro | >250 | 250 | 25 | 41.6 | 0.40 | 21.87 | 13.65 | 0.07 |

#In cases where the MBIC values were higher than the highest value tested (P128 or individual drugs, not in combination), the highest concentration was considered for the sake of calculation. This would give an underestimation of synergy and hence actual synergy will be even higher (i.e., lower FIC index) than shown here

Further, in order to find out if P128 would show synergy with SoC drugs possessing other mechanisms of action, we tested the synergy of P128 with linezolid (lin) and daptomycin (dap) on S. aureus MW2. As seen in Table 5, vancomycin and linezolid were poorly effective on biofilms whereas daptomycin showed relatively better activity. However, with all the three drugs there was a huge reduction in MBICs (8 to >128 fold) in the presence of P128 resulting in FIC index values ranging from 0.24 to 0.36.

TABLE 5
Synergy of P128 with vancomycin, linezolid and daptomycin on S. aureus
MW2 72 h preformed biofilms in 6 hr treatment assay

| | Vancomycin | | | | Linezolid | | | | Daptomycin | | | |
|---|---|---|---|---|---|---|---|---|---|---|---|---|
| S. | MIC (μg/mL) | | | | MIC (μg/mL) | | | | MIC (μg/mL) | | | |
| aureus | | | P128 + | | | | P128 + | | | | P128 + | |
| strain | P128 | van | van | FICI | P128 | lin | lin | FICI | P128 | dap | dap | FICI |
| MW2 | 6.25 | 250 | 1.5 + | 0.36 | 6.25 | >500 | 1.5 + | 0.24 | 6.25 | 31.2 | 1.5 + | 0.24 |
| | | | 15.6 | | | | 3.9 | | | | 3.9 | |

### VII. Scanning Electron Microscopy (SEM) of P128 Treated Biofilms

MRSA strain BK1 or MW2 was used for studying biofilm eradication activity of P128 by SEM. For this, biofilms formed in microtitre plates or on the surface of catheters were treated with increasing concentrations of P128 or antibiotics and subjected to SEM. S. aureus BK1 or MW2 was revived on blood agar plates and the culture was grown overnight in TSB supplemented with 20% glucose. The overnight-grown culture was diluted 1:50 in TSB with 2% glucose to achieve OD₆₀₀=˜0.1. 200 μL of the diluted culture was aliquoted into each well of the 96 well plate and incubated at 37° C. under static conditions for 18 h. After incubation the contents were aspirated, washed twice with 200 μL of PBS, and 100 μL of media+100 μL of drug were added to each well and incubated again at 37° C. for 18 h. The contents were aspirated, washed with 200 μL of PBS and allowed to dry at 37° C. for 15 min. The individual wells were cut out and biofilms visualized by SEM.

Results—

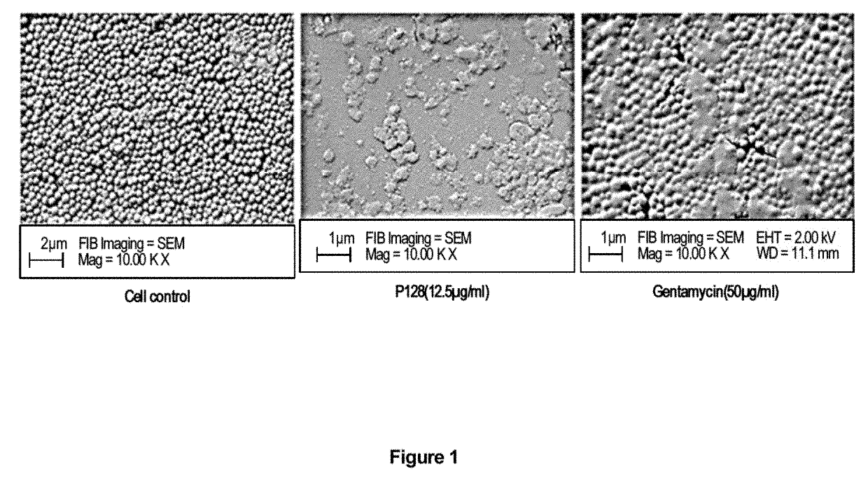

The bactericidal activity of P128 in biofilms was further confirmed by observing the treated biofilms by scanning electron microscopy (SEM). Observations of 72 h old biofilms by SEM showed that S. aureus BK1 formed thick biofilms on the surface of the microtitre plates (FIG. 1). Gentamycin at 50 μg/mL (>100× of planktonic cells MIC) did not have any appreciable effect on the appearance of the biofilm, and both the matrix and the embedded bacterial cells were seen to be intact. In contrast, P128 at the lowest concentration tested (12.5 μg/mL) destroyed the biofilm structure and lysed the bacterial cells completely, and no intact biofilm or cells were visible in a number of fields analyzed.

### VIII. Biofilm Eradication/Biomass Removal Activity of P128: Crystal Violet Staining Method

The protocol described by Schuch, et al. (2014) “Combination therapy with lysinCF-301 and antibiotic is superior to antibiotic alone for treating methicillin-resistant Staphylococcus aureus-induced murine bacteremia” J. Infect. Dis. 209:1469-78 was followed. Briefly, S. aureus MW2 strain grown on blood agar plates was inoculated into TSB with 2% glucose and the culture was grown overnight. The culture was diluted 1:50 in TSB to achieve an OD₆₀₀˜1.200 μL of the diluted culture was added to 1.8 mL of TSB media aliquoted into each well of a 24 well plate (˜5×10⁵ CFU). The plates were kept at 37° C. under static conditions for 18 h, the contents were aspirated, the wells were washed twice with 2.0 mLmL of 1×PBS, and 1 mLmL of media+1 mLmL of the drug was added to each well and incubated at 37° C. for 0, 2, 4 and 24 h. The contents were aspirated out at the stipulated time points and the wells were washed with 2 mL of 1×PBS. The wells were allowed to dry at 37° C. for 15 min and stained with 1 mLmL of 1% CV for 5 min. The wells were washed with 1 mLmL of 1×PBS, air dried and observed for intensity of blue color.

Quantification:

0.1% CV was used for staining biofilms. Post staining and washing steps, 1 mL 30% acetic acid is added to each well, incubated in RT for 5-10 min, contents in the well are mixed thoroughly and OD was read at 570 nm.

Results—

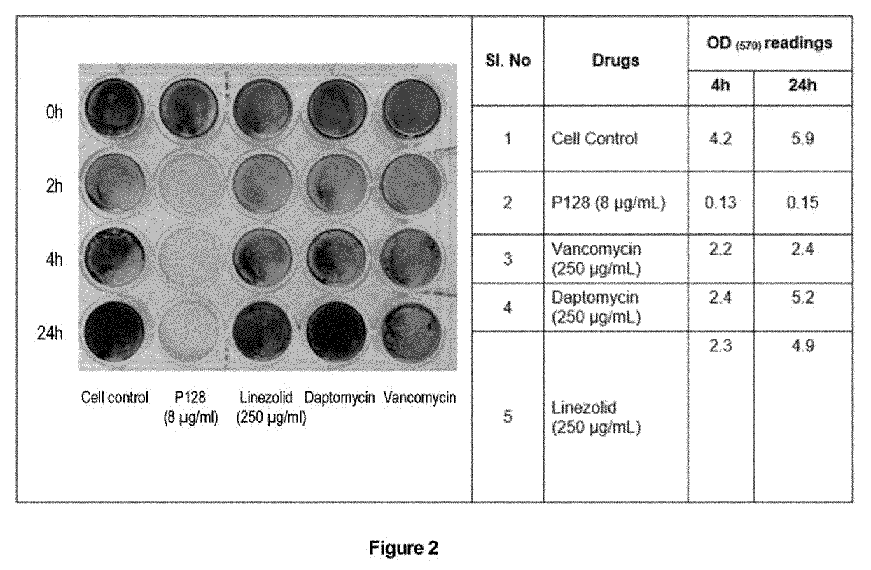

In the crystal violet staining assay, daptomycin, vancomycin and linezolid showed insignificant activity on 24 h preformed biofilm of MSSA and MRSA strains even after treatment at a high concentration (500 μg/mL) for 24 h (FIG. 2). In contrast P128 at 1×MIC (8 μg/mL) was able to eliminate the biofilms of the four strains tested within 2 h of treatment. This confirmed that P128 can eradicate an established biofilm of MRSA strain in a rapid manner. Eradication of biofilm using P128 was quantified by taking OD₅₇₀ readings for P128/Antibiotics treated and untreated wells. P128 treated well showed significant drop in OD as compared to cell control even after 24 h from (FIG. 2) indicating P128 activity on biofilm.

### IX. Biofilm Formation and Drug Treatment of Biofilms on Catheters:

To study the efficacy of P128 on biofilms on catheters, an overnight-grown culture of S. aureus MW2 was diluted 1:40 in TSB containing 5% rabbit plasma. Catheter (JMS Infusion set) pieces of 1-2 cm size were cut, slit into two halves and added to the culture. The cultures with catheter pieces were incubated at 37° C. with shaking at 100 rpm for 24 h. Post incubation, the catheters were removed and rinsed twice in PBS to remove the adhering planktonic cells. The biofilms on catheters were challenged with 8, 4, 2 and 1 μg/mL of P128 or 15, 30 and 90 μg/mL of vancomycin or 10 μg/mL of daptomycin (with 50 μg/mL CaCl₂) by transferring the catheter pieces into tubes containing the drugs. The tubes were incubated at 37° C. under static conditions for 18 hrs. The catheters were then removed from the tubes, rinsed once in PBS, and immersed in 0.1% safranin for 5 min. The stained catheters were washed once in PBS and allowed to dry. After drying, samples were fixed on aluminum stubs with double sided carbon adhesive tape, coated with 5-7 nm thickness gold using a sputter-coating system (Quorum Technologies; Q150T) and examined by SEM (Carl Zeiss; Neon 40) for the presence of biofilm structures.

Results—P128 Eliminates Preformed Biofilms from the Surface of Catheters

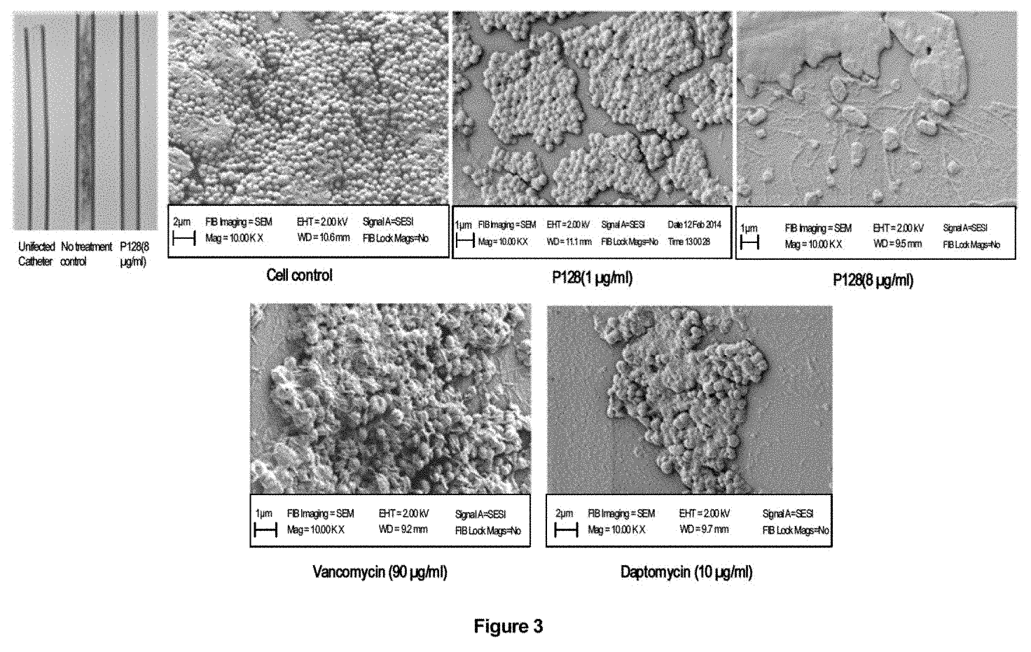

In order to simulate the in vivo conditions for biofilm formation in device associated infections, S. aureus was allowed to form biofilms on the surface of catheters. Because the conditions used for S. aureus biofilm formation in microtitre plates did not yield any biofilms on catheter surface, 5% hemolyzed plasma, a blood component used for getting luxuriant biofilms in vitro (Sun, et al. (2008) “In vitro multispecies Lubbock chronic wound biofilm model” Wound Repair Regen. 16:805-13), was added to the culture medium. This modification led to formation of robust biofilm by the MRSA MW2 strain as detected by safranin staining and by SEM (FIG. 3). Treatment of biofilms at 1×MIC concentration (8 μg/mL) led to eradication of the biofilm as no biofilm was visible upon safranin staining (FIG. 3). Similar observations were made by visualization of P128 treated biofilms by SEM wherein it was observed that P128 used at 1×MIC eradicated biofilms from the surface of catheters whereas vancomycin at 90 μg/mL (90×MIC) had a minimal effect on the structure of the biofilm (FIG. 3). Biofilms treated with daptomycin at 10×MIC (10 μg/mL) showed significant reduction in biofilm mass, though some intact patches of biofilm could be visualized Thus, both safranin staining assays and SEM observations confirmed that P128 possesses potent anti-biofilm activity on MRSA biofilms growing on catheters.

X. Bactericidal Activity of P128 on S. aureus Biofilm

In order to determine whether P128 could also act as a bactericidal agent in biofilms, the CFU reduction were monitored in S. aureus ATCC 29213 72 h preformed biofilms treated with various concentrations of P128 for 6 h. For CFU enumeration in catheter biofilm, the catheters were removed from the tubes, rinsed twice in PBS and placed in eppendorf tubes containing 1 mL PBS. To release the adhered biofilm into PBS, the catheters were scraped using inoculation loop. The samples were vortexed thoroughly and plated on LB agar plates.

Results—

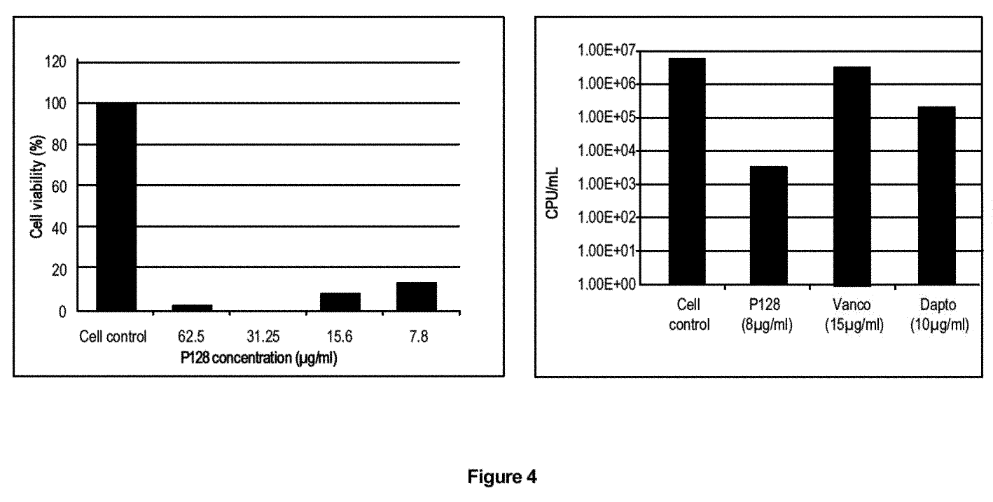

As shown in FIG. 4A, treatment of S. aureus ATCC 29213 biofilm with P128 showed dose dependent killing of S. aureus cells. During the incubation period there was very slow growth of bacteria, resulting in approximately 1 log CFU increase in untreated cultures in six hours. P128 at 7.8 μg/mL killed >90% of the cells, while exposure of biofilms to P128 concentrations greater than 31 μg/mL led to 99.9% cell killing of S. aureus cells. The bactericidal effect of 8 μg/mL (1×MIC) of P128 on S. aureus MW2 cells growing in biofilms on the surface of catheters led to 3 log reduction in CFU counts (FIG. 4B). Under similar conditions daptomycin at 10 μg/mL (10×MIC) showed 1 log CFU reduction, while vancomycin at 15 μg/mL (15×MIC) showed no significant effect on S. aureus viability.

### XI. Lubbock Chronic Wound Pathogenic Biofilm (LCWPB)—Prevention of Biofilm Formation by P128 in a Mixed Culture Model Simulating Chronic Wounds In Vitro

LCWPB media (Bolton broth, Oxoid Ltd, supplemented with 50% Bovine Plasma and 5% hemolyzed horse blood) was used for multi-species biofilm formation according to the procedure described. Sun, et al. (2008) “In vitro multispecies Lubbock chronic wound biofilm model” Wound Repair Regen. 16:805-13. Briefly, P. aeruginosa PAO1, E. faecalis ATCC 29212 and S. aureus ATCC 700699 grown on TSB agar plates were inoculated into TSB broth and grown at 37° C. in a shaker for 16 h. The cultures were individually diluted to 1×10⁶ CFU/mL, mixed in equal volumes, and 10 μL was added to 3 mL of LCWPB media containing a sterile pipette tip. For biofilm formation either two (P. aeruginosa PAO1, and S. aureus ATCC 700699) or all three bacterial species (P. aeruginosa PAO1, E. faecalis ATCC 29212 and S. aureus ATCC 700699) were inoculated into LCWPB media. In this model the pipette tip acts as a surface for biofilm formation. To test the ability of P128 to prevent biofilm formation, P128 at 10, 50 and 250 μg/mL was added to the tubes and the tubes were incubated at 37° C. in a shaker for 24 h with shaking at 150 rpm. Upon completion of incubation, the tips were removed from the tubes and placed on petri plates for observation. In the absence of P128, a confluent and thick mass of biofilm could be seen. The mass of the biofilm was greater in culture tubes with 3 species than in the ones with 2 species. For enumeration of bacteria in biofilms, the biofilm formed on the tips was washed twice in PBS, transferred into clean test tubes, and again washed twice with PBS. The washed biofilm mass was then transferred to a 50 mL conical polypropylene tube and the biofilm was macerated with sterile scissors. In situations when biofilm formation was not visible, the tips alone were processed as described above. The contents were vortexed thoroughly, diluted and plated on TSA plates. The plates were incubated for 24 h at 37° C. followed by incubation at ambient temperature for 24 h to enhance pigment production.

Results—

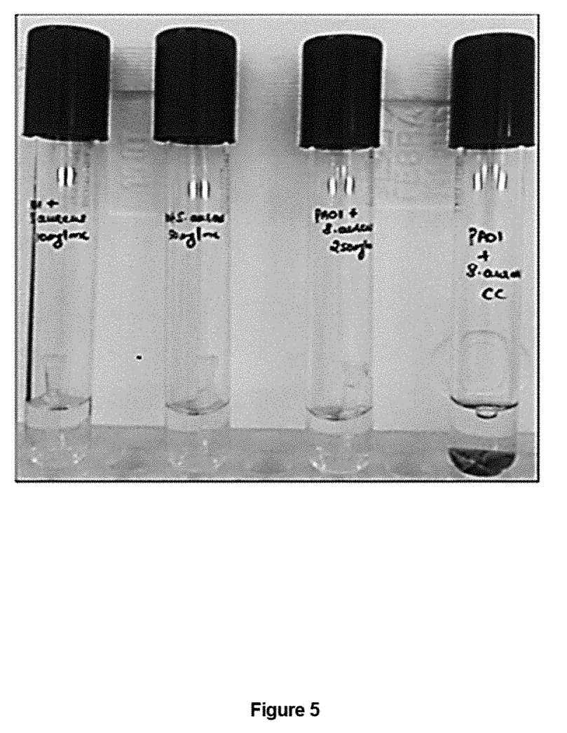

Chronic wounds such as venous leg ulcers are often infected with multiple species of Gram positive and Gram negative bacteria residing in a biofilm. Burmolle, et al. (2010) “Biofilms in chronic infections—a matter of opportunity—monospecies biofilms in multispecies infections” FEMS Immunol. Med. Microbiol. 59:324-36; and Wolcott, et al. (2013) “The polymicrobial nature of biofilm infection” Clin. Microbiol. Infect. 19:107-12. An in vitro model which uses plasma and leaked blood for the growth of S. aureus, P. aeruginosa and E. faecalis either singly or in mixed cultures allowing biofilm formation using a solid support, has been described. See Sun, et al. (2008) “In vitro multispecies Lubbock chronic wound biofilm model” Wound Repair Regen. 16:805-13. This model is supposed to mimic the wound environment under in vitro conditions. The ability of P128 to prevent biofilm formation in the mixed culture biofilm model was tested by the procedure described in materials and methods. The combination of either S. aureus and P. aeruginosa, or of S. aureus, P. aeruginosa and E. faecalis cultures led to the formation of a thick and leathery biofilm (FIG. 5) carrying an approximately equal number (10⁷-10⁸ CFU/mL) of all the organisms (Table 6). P128 at a concentration as low as 1×MIC (10 μg/mL) prevented the formation of biofilms in this model. The lack of biofilm formation was reflected in very low bacterial counts of P. aeruginosa, E. faecalis and S. aureus obtained after processing the pipette tips used for growing biofilms. At 50 and 250 μg/mL of P128, there was further reduction in S. aureus counts, whereas the counts of P. aeruginosa and E. faecalis remained at 10⁴ to 10⁵ CFU/mL. Since P128 does not inhibit the growth of E. faecalis and P. aeruginosa, these results suggest that inhibition of S. aureus growth alone in this model is sufficient to prevent biofilm formation even by P. aeruginosa and E. faecalis. Paul, et al. (2011) “A novel bacteriophage Tail-Associated Muralytic Enzyme (TAME) from Phage K and its development into a potent antistaphylococcal protein” BMC Microbiol. 11:226. This is consistent with the results of earlier studies involving inhibition of S. aureus and P. aeruginosa by various biofilm inhibitors in the same model of in vitro biofilm formation. Dowd, et al. (2009) “Effects of biofilm treatments on the multi-species Lubbock chronic wound biofilm model” J. Wound Care 18:510-12.

TABLE 6
Prevention of multi-species biofilm formation by P128 by inhibiting
growth of S. aureus. The cultures were treated with the indicated
concentrations of P128 and incubated for 24 h. Subsequently, the
pipette tips with or without biofilms were processed as described
in materials and methods and the CFU counts were recorded. The +
and − signs indicate presence and absence of a visible
biofilm on the surface of the pipette tip respectively. The experiment
was repeated three times with similar results and CFU counts from
one of the experiments have been shown here

| | | | |
|---|---|---|---|
| | | | Biofilm |
| P128 conc. μg/mL | Isolates | CFU/mL | formation |

Biofilm formation with P. aeruginosa
PAO1, S. aureus ATCC 700699

| | | | |
|---|---|---|---|
| 0 (Cell | P. aeruginosa | 2.4 × 10⁸ | + |
| control) | S. aureus | 2.1 × 10⁷ | |
| 10 | P. aeruginosa | 2 × 10⁶ | − |
| | S. aureus | 2.2 × 10⁵ | |
| 50 | P. aeruginosa | 1.5 × 10⁵ | − |
| | S. aureus | 1.8 × 10⁵ | |
| 250 | P. aeruginosa | 8 × 10⁵ | − |
| | S. aureus | 2 × 10³ | |

Biofilm formation with P. aeruginosaPAO1,
S. aureus ATCC 700699, E. faecalis ATCC29212

| | | | |
|---|---|---|---|
| 0 (cell | P. aeruginosa | 7 × 10⁸ | + |
| control) | S. aureus | 1 × 10⁸ | |
| | E. faecalis | 3 × 10⁷ | |
| 10 | P. aeruginosa | 2.2 × 10⁵ | − |
| | S. aureus | 2 × 10⁴ | |
| | E. faecalis | 9 × 10⁵ | |
| 50 | P. aeruginosa | 7 × 10⁴ | − |
| | S. aureus | 2 × 10³ | |
| | E. faecalis | 1.8 × 10⁴ | |
| 250 | P. aeruginosa | 1.7 × 10⁵ | − |
| | S. aureus | <10 | |
| | E. faecalis | 1.6 × 10⁴ | |

Activity of P128 on Coagulase Negative Staphylococcus (CoNS) Sp., S. epidermidis, S. lugdunensis, and S. haemolyticus
XII. Synergy of P128 with SOC Antibiotics—Planktonic Bacteria—MIC and Drug Combination Studies by Checkerboard Assays
P128 Synergy with Daptomycin and Oxacillin Using S. epidermidis Strains

S. epidermidis culture at a final cell number of 5×10⁵ CFU/mL was added to wells of 96-well microtiter plates (precoated with 0.5% BSA), containing two-fold dilutions of P128 and either Daptomycin or Oxacillin in cation adjusted Mueller Hinton Broth (CAMHB). CAMHB was supplemented with 50 g/mL Ca⁺⁺ for daptomycin assay. The plates were incubated at 37° C. for 24 h and the individual MICs and the combination MICs were read. The fractional inhibitory concentration index (FICI) was determined using the following equation: FICI=(MIC of drug A in the combination/MIC of drug A alone)+(MIC of drug B in the combination/MIC of drug B alone). The combination was considered to be synergistic when the FICI was ≤0.5; additive when FICI was between 0.5-1.0; indifferent when FICI was between 1-4 and antagonistic when FICI was ≥4. The experiments were performed in triplicate and repeated twice.

Results—

The MIC of P128 on these strains was found to range from 2-32 μg/mL, while the MIC of Daptomycin was 0.5-2 μg/mL (Table 7). Similarly the MIC of Oxacillin was 16 μg/mL 0.12 μg/mL on the sensitive strains and on resistant strains respectively (Table 8). Both Daptomycin and Oxacillin in combination with P128 showed a clear synergistic effect, with FIC index ranging from 0.09 to 0.5.

TABLE 7
P128 & Daptomycin synergy on S. epidermidis strains

| | | MIC(μg/mL) | | | | |
|---|---|---|---|---|---|---|
| Sl. | | | | P128 + | | |
| No. | Isolates | P128 | Daptomycin | Daptomycin | FICI value | |
| 1 | B9471 | 8 | 2 | 1.0 + 0.5 | 0.37 | (Synergy) |
| 2 | B9472 | 16 | 2 | 2.0 + 0.5 | 0.28 | (Synergy) |
| 3 | B9467 | 32 | 2 | 8 + 0.5 | 05 | (Synergy) |
| 4 | B9468 | 2 | 0.5 | 0.12 + 0.25 | 0.5 | (Synergy) |
| 5 | ATCC | 4 | 1 | 1 + 0.25 | 0.5 | (Synergy) |
| | 12228 | | | | | |

TABLE 8
P128 & Oxacillin synergy on S. epidermidis strains

| | | MIC (μg/mL) | | | | |
|---|---|---|---|---|---|---|
| Sl. | | | | P128 + | | |
| No. | Isolates | P128 | Oxacillin | Oxacillin | FICI value | |
| 1 | B9470 | 8 | 16 | 1.0 + 0.5 | 0.15 | (Synergy) |
| 2 | B9471 | 8 | 16 | 2.0 + 0.5 | 0.28 | (Synergy) |
| 3 | B9472 | 8 | 16 | 1.0 + 0.25 | 0.14 | (Synergy) |
| 4 | B9473 | 16 | 16 | 4.0 + 0.5 | 0.28 | (Synergy) |
| 5 | B9467 | 32 | 8 | 2.0 + 0.25 | 0.09 | (Synergy) |
| 6 | B9468 | 2 | 0.12 | 0.5 + 0.03 | 0.5 | (Synergy) |

XIII. Synergy of P128 with SOC Antibiotics—Biofilm Model—MIC and Drug Combination Studies by Checkerboard Assays

### Standardization of Biofilm Formation Conditions

Culture conditions were optimized for reproducibly obtaining a robust biofilm of S. epidermidis in 96 well microtitre plates. For this purpose, biofilms were generated in microtiter plates and the surface-adhered cultures remaining after washing off the planktonic cells were analyzed at the end of 72 h by MTT dye assay. Briefly, an overnight-grown culture of the S. epidermidis strain was diluted 1:40 in LB broth. 200 μL of diluted culture was aliquoted into microtiter plate wells. Microplates were placed in a shaker-incubator set to 37° C., and 100 rpm for 24 h followed by 48 h incubation under static conditions at 37° C. The contents of wells were aspirated and discarded. Wells were washed twice with 1×PBS and the presence of biofilm in the wells at the end of 72-hour period was determined by metabolic dye-reduction assay method using MTT (3-(4,5-dimethylthiazol-2-yl)-2,5-diphenyltetrazolium bromide; Himedia). In this assay, live cells reduce the dye leading to color formation which can be read at 570 nm and the intensity of color can be correlated to number of live cells.

P128 Synergy with Antibiotics—Biofilm Model

For testing if P128 showed synergy with other drugs, combinations of P128 and antibiotics were tested by the checkerboard method. For synergy studies, the wells were challenged with various concentrations of P128 or other antibiotic drugs in LB and incubated for 24 h at 37° C. LB supplemented with 50 μg/mL Ca⁺⁺ was used for daptomycin treatment wells. The contents of the well was aspirated out and discarded. The biofilm adhered to the wells was quantified by MTT assay as described above. In each experiment, in addition to the combination MBIC, the MBIC of each drug was also determined individually. The fractional MBIC concentrations were determined by MTT dye method as described above. The FICI and synergy was calculated.

Results—

The MBIC values of P128 and the antibiotics in combination were much reduced compared to their individual MBIC values on all three strains of S. epidermidis. The FIC indices of P128 combinations with the antibiotics ranged from 0.035 to 0.49, suggesting a strong synergistic mechanism of inhibition (Table 9).

TABLE 9
Synergy of P128 with vancomycin, linezolid and daptomycin on
S. epidermidis 72 h preformed biofilms in 6 hr treatment assay

| | S. epidermidis | | | P128 + Vancomycin μg/mL | | |
|---|---|---|---|---|---|---|
| Sl. No | Strain | P128 μg/mL | Vancomycin μg/mL | P128 | Vancomycin | FICI |
| 1 | B9470 | 32 | 7.8 | 3.9 | 1.9 | 0.36 |
| 2 | B9471 | 32 | 250 | 0.9 | 15.6 | 0.08 |
| 3 | B9472 | 32 | 250 | 7.8 | 62.5 | 0.49 |
| | | | | P128 + Linezolid μg/mL | | |
| | | P128 μg/mL | Linezolid μg/mL | P128 | Linezolid | FICI |
| 1 | B9470 | 32 | 7.8 | 4 | 0.45 | 0.17 |
| 2 | B9471 | 32 | 250 | 4 | 0.9 | 0.12 |
| 3 | B9472 | 32 | 250 | 1 | 0.9 | 0.035 |
| | | | | P128 + Daptomycin μg/mL | | |
| | | P128 μg/mL | Daptomycin μg/mL | P128 | Daptomycin | FICI |
| 1 | B9470 | 16 | 15.6 | 1 | 3.9 | 0.31 |
| 2 | B9471 | 32 | 15.6 | 1 | 1.9 | 0.15 |
| 3 | B9472 | 32 | 7.8 | 1 | 0.9 | 0.14 |

TABLE D1
Synergy of P128 with vancomycin, linezolid and daptomycin
on 72 h preformed biofilms in 6 hr treatment
S. lugdunensis B9510

| | | | | P128 + Linezolid μg/mL | | |
|---|---|---|---|---|---|---|
| Sl. no | Combination | P128 μg/mL | Linezolid μg/mL | P128 | Linezolid | FICI |
| 1 | P128 + linezolid | 16 | 125 | 2 | 1.9 | 0.13 |
| | | | | P128 + Vancomycin μg/mL | | |
| | | P128 μg/mL | Vancomycin μg/mL | P128 | Vancomycin | |
| 2 | P128 + Vancomycin | 32 | 250 | 1 | 7.8 | 0.06 |
| | | | | P128 + Daptomycin μg/mL | | |
| | | P128 μg/mL | Daptomycin μg/mL | P128 | Daptomycin | |
| 3 | P128 + Daptomycin | 16 | 15.6 | 2 | 3.9 | 0.37 |

S. haemolyticus B9511

| | | | | P128 + Linezolid μg/mL | | |
|---|---|---|---|---|---|---|
| Sl. no | Combination | P128 μg/mL | Linezolid μg/mL | P128 | Linezolid | FICI |
| 1 | P128 + linezolid | 6.25 | >250 | 1.56 | 1.9 | 0.24 |
| | | | | P128 + Vancomycin μg/mL | | |
| | | P128 μg/mL | Vancomycin μg/mL | P128 | Vancomycin | |
| 2 | P128 + Vancomycin | 12.5 | >250 | 1.56 | 15.6 | 0.18 |
| | | | | P128 + Daptomycin μg/mL | | |
| | | P128 μg/mL | Daptomycin μg/mL | P128 | Daptomycin | |
| 3 | P128 + Daptomycin | 12.5 | >250 | 1.56 | 15.6 | 0.18 |

### XIV. P128 Eradicates CoNS Biofilms

The protocol described earlier by Schuch, et al. (2014) “Combination therapy with lysinCF-301 and antibiotic is superior to antibiotic alone for treating methicillin-resistant Staphylococcus aureus-induced murine bacteremia” J. Infect. Dis. 209:1469-78 was followed. Briefly, S. epidermidis, S. lugdunensis and S. haemolyticus strains were inoculated into TSB with 2% glucose and the cultures were grown overnight. The cultures were diluted 1:50 in TSB to achieve an OD₆₀₀˜0.1. Two hundred micro liter of the diluted culture was added to 1.8 ml of TSB media aliquoted into each well of a 24 well plate (˜5×10⁵ CFU). The plates were kept at 37° C. under static conditions for 18 h, the contents were aspirated, the wells were washed twice with 2.0 ml of 1×PBS, and 1 mL of media+1 mL of the drug was added to each well and incubated at 37° C. for 0, 2, 4, and 24 h. TSB supplemented with 50 μg/ml Ca⁺⁺ was used for daptomycin treatment wells. The contents were aspirated out at the stipulated time points and the wells were washed with 2 ml of 1×PBS. The wells were allowed to dry at 37° C. for 15 min and stained with 1 mL of 1% CV for 5 min. The wells were washed with 1 mL of 1×PBS, air dried and observed for intensity of blue color.

Quantification—

0.1% CV was used for staining biofilms. Post staining and washing steps, 1 mL 30% acetic acid is added to each well, incubated in RT for 5-10 min, contents in the well are mixed thoroughly and OD was read at 570 nm.

Results—

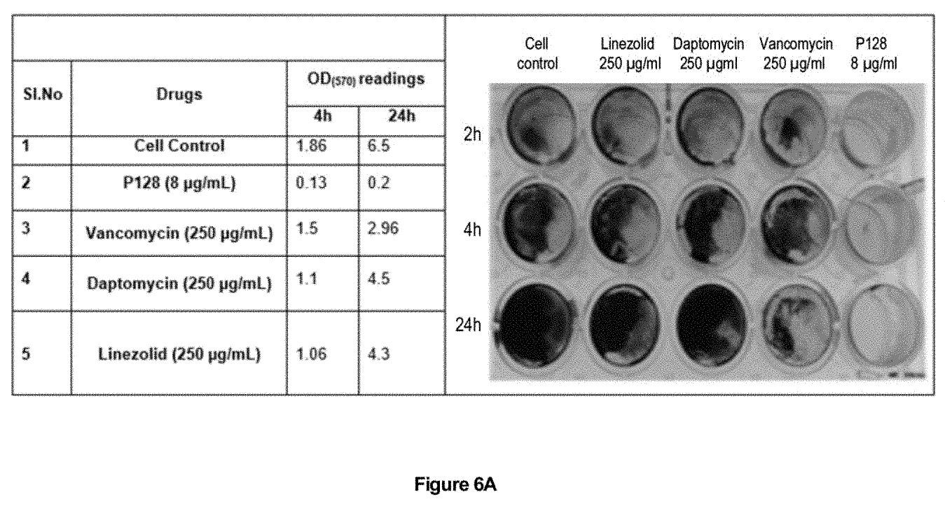

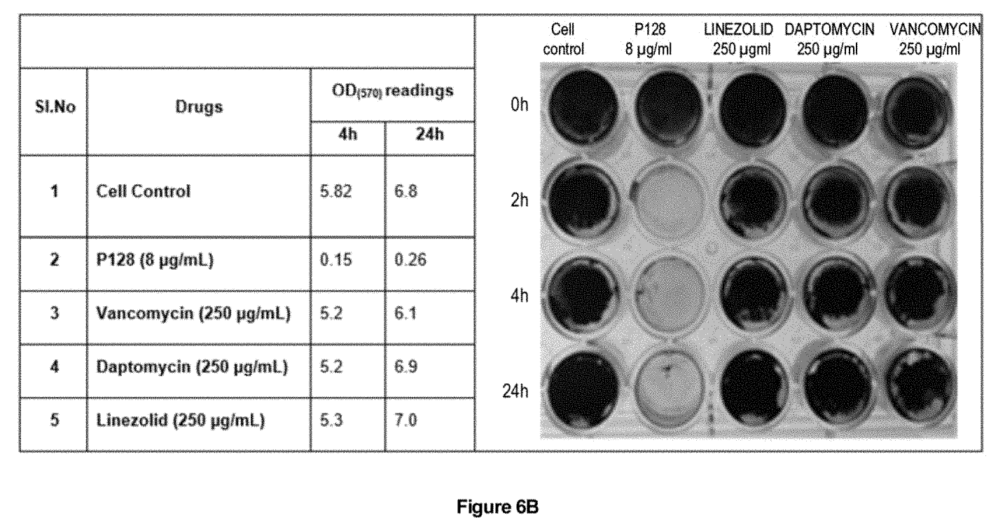

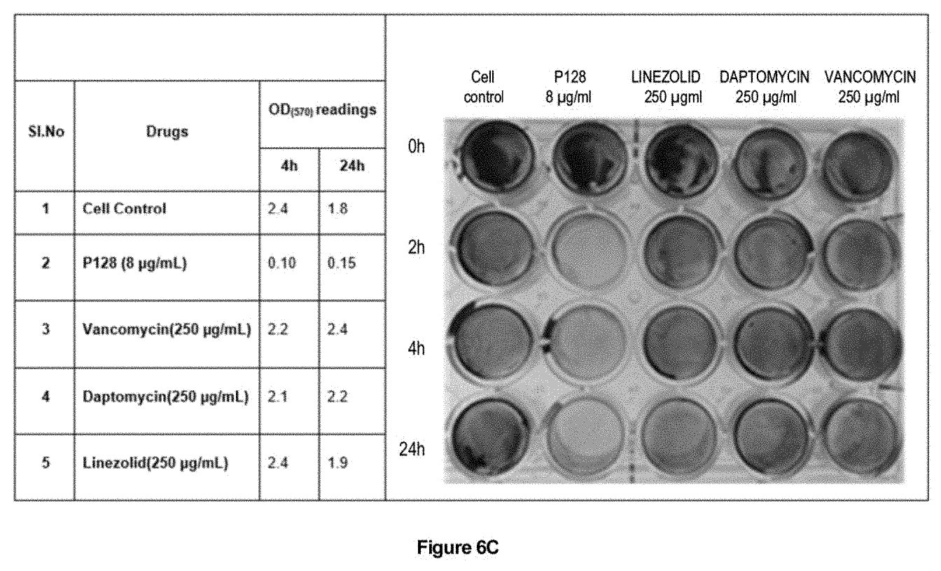

Daptomycin, vancomycin, and linezolid showed poor activity on preformed biofilm of all the three CoNS tested even after treatment of the biofilm at a high concentration (250 and 100 μg/mL) for 4 h. In contrast. P128 at 1×MIC (8 μg/mL) was able to eliminate the biofilms within 2 h of treatment. Eradication of biofilm using P128 was quantified by taking OD₅₇₀ readings for P128/Antibiotics treated and untreated wells. P128 treated well showed significant drop in OD as compared to cell control even after 24 h from (FIGS. 6 A, B, and C) indicating P128 has activity on biofilm. The drop in OD readings was seen with all the three CoNS sp., viz., S. epidermidis, S. lugdunensis, and S. haemolyticus. This confirmed that P128 can eradicate an established biofilm of CoNS strains in a rapid manner.

### XV. Biofilm Formation and Drug Treatment of Biofilms on Catheters

In order to simulate the in vivo conditions for biofilm formation in device associated infections, S. epidermidis B9470 was allowed to form biofilms on the surface of catheters. An overnight-grown culture was diluted 1:40 in TSB containing 4% sodium chloride. Catheter (JMS Infusion set) pieces of 1-2 cm size were cut, slit into two halves and added to the culture. The cultures with catheter pieces were incubated at 37° C. with shaking at 100 rpm for 42 h. Post incubation, the catheters were removed and rinsed twice in PBS to remove the adhering planktonic cells. The biofilms on catheters were challenged with 8 μg/mL of P128 or 30 μg/mL of vancomycin by transferring the catheter pieces into tubes containing the drugs. The tubes were incubated at 37° C. under static conditions for 18 hrs. The catheters were then removed from the tubes, rinsed once in PBS, and immersed in 0.1% safranin [a dye that stains cell wall of bacteria] for 5 min. The stained catheters were washed once in PBS and allowed to dry. After drying, samples were fixed on aluminum stubs with double sided carbon adhesive tape, coated with 5-7 nm thickness gold using a sputter-coating system (Quorum Technologies; Q150T) and examined by SEM (Carl Zeiss. Neon 40) for the presence of biofilm structures.

Results—

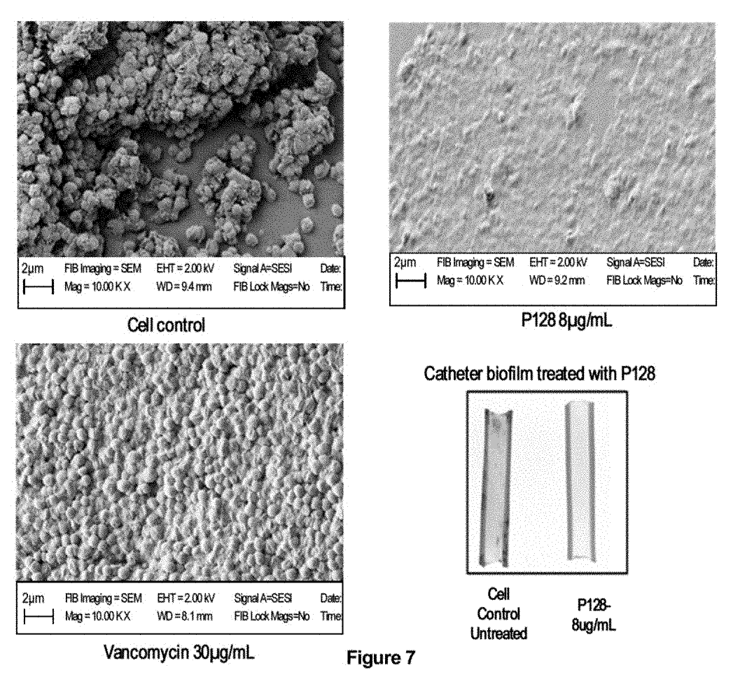

Treatment of biofilms at 1×MIC concentration (8 μg/mL) led to eradication of the biofilm as no biofilm was visible upon safranin staining (FIG. 7). Similar observations were made by visualization of P128 treated biofilms by SEM wherein it was observed that P128 used at 1×MIC eradicated biofilms from the surface of catheters whereas vancomycin at 30 μg/mL (30×MIC) had a minimal effect on the structure of the biofilm (FIG. 7). Thus, both safranin staining assays and SEM observations confirmed that P128 possesses potent anti-biofilm activity on S. epidermidis biofilms growing on catheters.

### Biofilm Formation and Drug Treatment of Biofilms on Catheters—Additional Data:

Method:

In order to simulate the in vivo conditions for biofilm formation in device associated infections, S. haemolyticus B9478, or S. lugdunensis B9510 was allowed to form biofilms on the surface of catheters. An overnight-grown culture was diluted 1:40 in TSB containing 4% sodium chloride for S. lugdunensis B9510 strain and 1% sodium chloride and 3% glucose for S. haemolyticus B9478. Catheter (JMS Infusion set) pieces of 1-2 cm size were cut, slit into two halves and added to the culture. The cultures with catheter pieces were incubated at 37° C. with shaking at 100 rpm for 42 h. Post incubation, the catheters were removed and rinsed twice in PBS to remove the adhering planktonic cells. The biofilms on catheters were challenged with 8 μg/ml of P128 or 30 μg/ml of vancomycin by transferring the catheter pieces into tubes containing the test agents. The tubes were incubated at 37° C. under static conditions for 18 his. The catheters were then removed from the tubes, rinsed once in PBS, and allowed to dry. After drying, samples were fixed on aluminum stubs with double sided carbon adhesive tape, coated with 5-7 nm thickness gold using a sputter-coating system (Quorum Technologies; Q150T) and examined by SEM (Carl Zeiss; Neon 40) for the presence of biofilm structures.

Results:

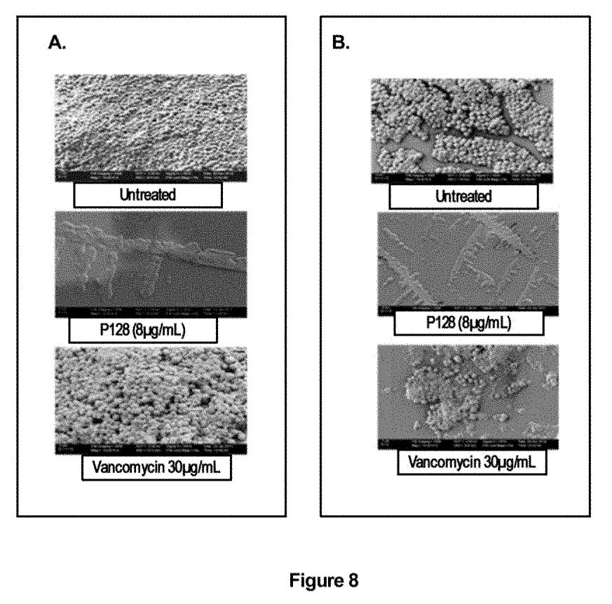

Treatment of biofilms at 1×MIC concentration (8 μg/ml) led to eradication of the biofilm as no biofilm was visible. Observations were made by visualization of P128 treated biofilms by SEM wherein it was observed that P128 used at IX MIC eradicated biofilms from the surface of catheters whereas vancomycin at 30 μg/ml (30×MIC) had a minimal effect on the structure of the biofilm. Thus, SEM observations confirmed that P128 possesses potent anti-biofilm activity on S. lugdunensis (A) and S. haemolyticus (B) biofilms growing on catheters (FIG. 8).

### XVI. Activity of P128 on Antibiotic Persisters of CoPS and CoNS

Persisters are not mutants, but rather dormant cells that can survive the antimicrobial treatments that kill the majority of their genetically identical siblings. They are phenotypic variants of actively dividing cells produced stochastically in the population, and their relative abundance rises—reaching 1%—at the late-exponential phase of growth. Persisters are non-growing dormant cells, which explains their tolerance to bactericidal antibiotics that depend on the presence of active targets for killing the cell.

Generation of Persisters of Daptomycin and Vancomycin for S. aureus Strains BK18 and B9377 and S. epidermidis Strain B9470

Persisters of S. aureus BK18 and B9377 and S. epidermidis strain B9470 were generated as per protocol described by Lechner, et al (Staphylococcus aureus persisters tolerant to bactericidal antibiotics. Lechner, et al. (2012) J Mol Microbiol Biotechnol; 22(4):235-44). Briefly, colonies were suspended in LB broth and allowed to grow at 37° C., 200 rpm for ˜2 hours. The cultures were pelleted, resuspended in MHB and OD₆₀₀ was adjusted to 0.5 to 1.0 OD (˜2 to 5×10⁸ CFU/mL). 2.7 mL of this culture was aliquoted to test tubes and 300 μL of antibiotic was added at specific concentrations (10×, 50×, or 100×MIC). The test tubes were incubated at 37° C., 200 rpm, and 250 μL was sampled out at 4, 8, and 24 hr time points. This aliquot was pelleted, washed in saline and resuspended in equal volume of saline, diluted and plated on LB agar. Rapid decrease in CFUs, followed by rather stable values for up to 24 hrs, indicated the presence of Daptomycin/Vancomycin tolerant persisters.

### Confirmation of Persisters

After sampling for P128 and antibiotic activity, the rest of the sample was pelleted, washed in saline and resuspended into fresh media (LB). Allowed to grow at 37° C., 200 rpm until it reaches 1 OD and then the same steps followed as done for generation of persisters. A biphasic growth curve similar to that obtained during generation of persisters would indicate the tolerance to be phenotypic and not genotypic.

### P128 and Antibiotic Activity on Persisters

Persisters were generated as above were treated with P128 and antibiotics (Ref: Gutiérrez, et al. (2014) Effective Removal of Staphylococcal Biofilms by the Endolysin LysH5 PLoS One 9(9): e107307).

To 450 μL aliquots, P128. Daptomycin and Vancomycin (in saline) were added (individually) each at 1×MIC Conc., and another 450 μL aliquot was taken as control. The persister samples treated with P128 were incubated for 1 h, 37° C., and 200 rpm and those treated with antibiotics were incubated for 6 h, 37° C., and 200 rpm. After incubation samples were serially log diluted and plated.

Results—Generation of Persisters of Daptomycin and Vancomycin for S. aureus Strains BK18 and B9377 and S. epidermidis Strain B9470—

Approximately two log drop in CFU was observed at the end of 4 h treatment with Vancomycin that continued to 24 h. Daptomycin treatment yielded 5 log drop in CFUs at the end of 4 h and continued to be the same to 24 h. This rapid decrease in CFUs, (in presence of antibiotics) followed by rather stable values for up to 24 h, indicated the presence of Daptomycin/Vancomycin tolerant persisters (Table 10).

TABLE 10
Generation of persisters of Daptomycin and Vancomycin

| Time (h) | Cell Control | Vancomycin 100X | Daptomycin 50X |
|---|---|---|---|
| | BK18 (CFU/mL) | | |
| 0 | 2 × 10⁸ | 2 × 10⁸ | 2 × 10⁸ |
| 4 | 4 × 10⁸ | 1 × 10⁷ | 3 × 10³ |
| 8 | 8 × 10⁸ | 2 × 10⁶ | 1 × 10³ |
| 24 | 1 × 10⁹ | 4 × 10⁵ | 5 × 10³ |
| | B9377 (CFU/mL) | | |
| 0 | 2.5 × 10⁸ | 2.5 × 10⁸ | 2.5 × 10⁸ |
| 4 | 5 × 10⁸ | 8 × 10⁶ | 4 × 10³ |
| 8 | 8 × 10⁸ | 1.9 × 10⁶ | 1 × 10³ |
| 24 | 2 × 10⁹ | 1.1 × 10⁵ | 5.5 × 10³ |

| | B9470 (CFU/mL | | |
|---|---|---|---|
| | Cell Control | Vancomycin 50X | Daptomycin 10X |
| 0 | 3.2 × 10⁷ | 3.2 × 10⁷ | 3.2 × 10⁷ |
| 2 | 1.2 × 10⁷ | 1 × 10⁶ | 1 × 10⁷ |
| 4 | 3 × 10⁷ | 3 × 10⁵ | 2.3 × 10⁶ |
| 8 | 1 × 10⁷ | 9 × 10³ | 5 × 10⁵ |
| 24 | 1.4 × 10⁸ | 3 × 10⁴ | 3 × 10⁶ |

### Confirmation of Persisters

Cells after incubation with antibiotics for 24 h, when washed and inoculated in fresh medium, the same death curve resulted—a biphasic growth curve similar to that obtained during the first step of generation of persisters, indicating the tolerance effect of the antibiotics for the cells rather than antibiotic resistance (see Table 11).

TABLE 11
Confirmation of persisters generated - Biphasic growth

| Time (h) | Cell Control | Vancomycin 100X | Daptomycin 50× |
|---|---|---|---|
| | BK18 (CFU/mL) | | |
| 0 | 1 × 10⁸ | 1 × 10⁸ | 1 × 10⁸ |
| 4 | 4 × 10⁸ | 2.5 × 10⁵ | 3 × 10² |
| 8 | 6 × 10⁸ | 1 × 10⁵ | 1 × 10² |
| 24 | 1 × 10⁹ | 1 × 10⁵ | 6 × 10² |
| | B9377 (CFU/mL) | | |
| 0 | 1.5 × 10⁸ | 1.5 × 10⁸ | 2.5 × 10⁸ |
| 4 | 5 × 10⁸ | 2 × 10⁶ | 6 × 10³ |
| 8 | 7 × 10⁸ | 2 × 10⁶ | 1 × 10³ |
| 24 | 1 × 10⁹ | 8 × 10⁵ | 9 × 10² |
| | B9470 (CFU/mL) | | |
| 0 | 1 × 10⁷ | 2 × 10⁷ | 1 × 10⁷ |
| 2 | 2.5 × 10⁶ | 1 × 10⁵ | 9 × 10⁴ |
| 4 | 3 × 10⁷ | 6 × 10⁴ | 4 × 10³ |
| 8 | 3 × 10⁷ | 1.5 × 10⁴ | 6 × 10² |
| 24 | 8 × 10⁷ | 5 × 10³ | 5 × 10³ |

### P128 and Antibiotic Activity on Persisters

P128 was active on antibiotic persisters of S. aureus BK18 and B9377. Vancomycin persisters (1×10⁶ CFU/mL) when treated with P128 showed ˜5 log drop in CFU while <10 CFU/mL were recovered after daptomycin persisters were treated with P128. The antibiotics did not show any activity on these persister cells as expected, except for BK 18 daptomycin persisters. Antibiotic persister cells of S. epidermidis strain B9470 when treated with P128 yielded 3 to 4 log drop in CFU indicating P128 activity on CoNS persisters (Table 12).

TABLE 12
P128 activity on persisters

| | Treated with | | |
|---|---|---|---|
| Celt Control | P128 | Vancomycin | Daptomycin |

P128 activity on BK18 Vancomycin persisters (CFU/mL)

| | | | |
|---|---|---|---|
| 1 × 10⁶ | 5 × 10¹ | 8 × 10⁵ | 7.5 × 10⁵ |

P128 activity on BK18Daptomycin persisters (CFU/mL)

| | | | |
|---|---|---|---|
| 1 × 10² | 1 × 10¹ | 1 × 10¹ | 1 × 10¹ |

P128 activity on B9377 Vancomycin persisters (CFU/mL)

| | | | |
|---|---|---|---|
| 1 × 10⁶ | 2.5 × 10¹ | 4 × 10⁵ | 6 × 10⁵ |

B9377 Daptomycin persisters (CFU/mL)

| | | | |
|---|---|---|---|
| 2.6 × 10² | 1 × 10¹ | 2.6 × 10² | 1 × 10² |

P128 activity on b9470Vancomycin persisted (CFU/mL)

| | | | |
|---|---|---|---|
| 6 × 10⁴ | 7 × 10¹ | 1.5 × 10³ | 2.1 × 10² |

P128 activity on B9470Daptomycin persisters (CFU/mL)

| | | | |
|---|---|---|---|
| 1 × 10⁶ | 2.5 × 10² | 5 × 10⁵ | 1.3 × 10⁶ |

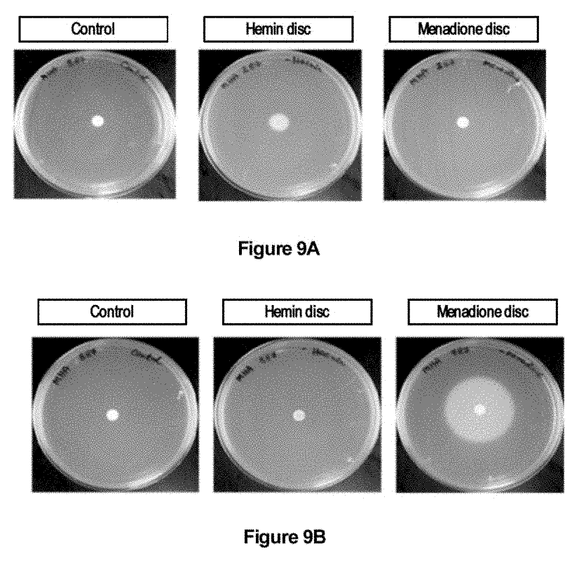

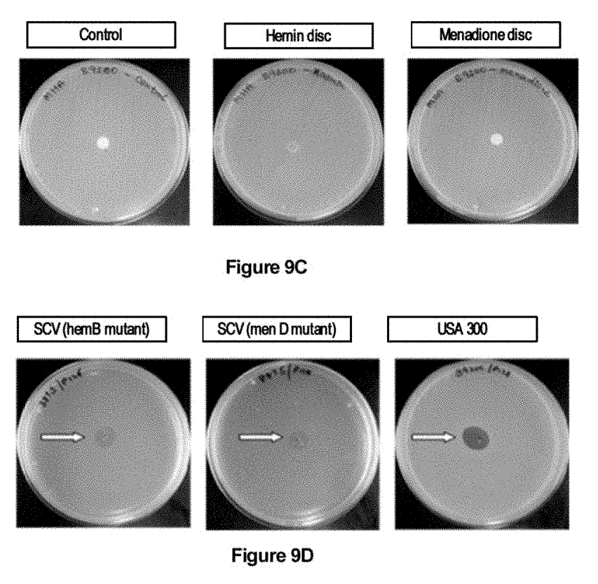

XVII. P128 is Effective in Killing Small Colony Variants (SCVs) of S. aureus (FIG. 9A-D)

One special feature of S. aureus infections is their chronic and recurrent nature despite appropriate antibiotic treatment. Within the last 20 years, many reports have described the association of such recurrent infections with the occurrence of SCVs of S. aureus, a special phenotype with attenuated virulence, thereby facilitating intracellular survival and evasion of the immune system (Kahl, et al. (2016) “Clinical significance and pathogenesis of staphylococcal small colony variants in persistent infections” Clin. Microbiol. Rev. 29:401-427).

Clinical S. aureus SCVs are frequently auxotrophic for menadione or hemin, two compounds that are involved in the biosynthesis of the electron transport chain components menaquinone and cytochromes, respectively (Von Eiff, et al. (2006) “Phenotype Microarray Profiling of Staphylococcus aureus menD and hemB Mutants with the Small-Colony-Variant Phenotype” J. Bacteriol. 188:687-693). We tested P128 activity on small colony variants of S. aureus by lawn inhibition assay.

Method: Characterization of SCV:

Two S. aureus small colony variants viz., hemB mutant and menD mutants were characterized for SCV phenotype and auxotrophy for hemin and menadione. Both the isolates were revived on LB agar with and without Erythromycin (5 μg/mL) and Blood agar medium and incubated at 37° C. for 48 hr.

Result:

Both the isolates showed colony morphology of small colony variants on LB agar with Erythromycin (5 μg/mL) with after 48 hours of incubation.

Auxotrophy Confirmation:

(FIGS. 9 A and B); Both the isolates were cultured in LB broth with Erythromycin (5 μg/mL) at 37° C. for 48 hr. 100 μL of the cultures were then swabbed on Muller Hinton Agar medium separately. A sterile paper disc (Himedia) was placed in the centre of the swabbed medium. 10 μL of Haemin (1 mg/mL in DMSO) or Menadione (1 mg/mL in water) was added separately on the disc. A control plate was maintained with sterile disc 10 μL of DMSO. The plates were incubated at 37° C. for 24 hr.

P128 Activity by Lawn Inhibition Assay:

P128 was tested on both the mutants by lawn inhibition assay and the SCV's were susceptible to P128.

### P128 Activity by CFU Drop Assay.

Method:

Bacterial cultures were grown in LB medium at 37° C. until OD₆₀₀ reached 1.0 and then diluted in LB to obtain 10⁷ CFU. 100 μL of cells (10⁷ CFU) in LB were treated with 100 μL of P128 (in saline) and incubated at 37° C. for 1 h, 200 rpm, cell controls without proteins were maintained. Following incubation, the volume was made up to 1 mL with LB broth, serially log diluted in LB broth and plated on LB agar. Incubated at 37° C. for 16-18 h to enumerate residual CFU and determine cell killing.

Results:

P128 is active on SCV was comparable to wild type strain. P128 showed more than 4 log reduction in CFU of SCV strains (see Table D4).

TABLE D4
CFU drop assay of P128 on SCVs

| Strains | | CFU/mL |
|---|---|---|
| hemB small colony mutant | Cell control | 2.8 × 10⁷ |
| NR48387 | P128 treated | 3.0 × 10¹ |
| menD mutant small colony | Cell control | 5.8 × 10⁷ |
| mutant | P128 treated | 1.2 × 10³ |
| S. aureus USA 300 | Cell control | 2.8 × 10⁸ |
| (wild type) | P128 treated | 4.5 × 10² |

### XVIII. P128 Activity on Stationary Phase Cells

Most in vitro studies examining the activity of antibiotics have been performed on rapidly dividing planktonic bacterial cultures supplemented with rich growth media. However, in infections, bacteria seldom encounter optimal conditions that allow logarithmic growth. It is likely that stationary-phase or non dividing bacteria are common in many persistent Staphylococcus infections such as endocarditis and osteomyelitis and in biofilm-associated infections (e.g., on catheters, grafts, and foreign bodies). Therefore, activity of P128 was determined on stationary phase cells of three S. aureus strains, three S. epidermidis, and two each of S. lugdunensis and S. haemolyticus strains. Briefly, the test cultures were grown overnight in LB (˜10⁹ cells), centrifuged, pellet was washed and resuspended in saline. Optical density (OD₆₀₀) was adjusted to 0.2 (˜10⁸ CFU/mL). 100 μL of these cells were treated with 10 μg/mL P128 (in saline) and incubated at 37° C. for 2 h, 200 rpm, cell controls without proteins were maintained. Following incubation, the volume was made up to 1 mL with saline, serially log diluted in saline and plated on LB agar. Incubated at 37° C. for 18 h to enumerate residual CFU to determine cell killing.

Results—

P128 was active on stationary cells (see Table 13). Three to five log drop in CFU was observed with P128 treated cells of S. aureus and coagulase negative S. epidermidis, S. lugdunensis, and S. haemolyticus demonstrating bactericidal action of P128 against stationary cells.

TABLE 13
P128 activity on stationary phase cells

| | | |
|---|---|---|
| Strains | Cell control | P128 treated |

S. aureus strains (CFU/mL)

| | | |
|---|---|---|
| BK18 | 5.5 × 10⁶ | 1 × 10¹ |
| BK31 | 2.9 × 10⁶ | 3.5 × 10¹ |
| B9377 | 5 × 10⁶ | 3 × 10² |

S. epidermidis strains (CFU/mL)

| | | |
|---|---|---|
| B9467 | 1 × 10⁶ | 3.7 × 10² |
| B9470 | 5 × 10⁶ | 1.5 × 10² |
| B9472 | 1.6 × 10⁶ | 7 × 10¹ |

S. lugdunensis strains (CFU/mL)

| | | |
|---|---|---|
| B9474 | 7.4 × 10⁶ | 4.9 × 10² |
| B9475 | 1.4 × 10⁶ | 2.7 × 10⁴ |

S. haemolyticus strains (CFU/mL)

| | | |
|---|---|---|
| B9478 | 6 × 10⁶ | 1.1 × 10³ |
| B9479 | 2.8 × 10⁶ | 6 × 10² |

### XIX. Serum Potentiates P128 Activity

Serum had a potentiating effect on P128 inhibition as the serum MIC values on 2 strains each of S. aureus, S. epidermidis, S. haemolyticus, and S. lugdunensis were reduced 4 to 64 fold compared to the MIC values in CAMHB (see Table D2).

TABLE D2
Comparison of P128 MIC values on CoNS strains in CAMHB and Serum

| Sl. | | | MIC of P128 in μg/ml (μM) | | | | Fold reduction in |
|---|---|---|---|---|---|---|---|
| No. | Strains | Isolate No. | CAMHB | | Serum | | serum MIC |
| 1 | S. aureus | MW2 | 8 | (0.29) | 0.12 | (0.004) | 64 |
| | | USA 300 | 8 | (0.29) | 0.12 | (0.004) | 64 |
| 2 | S. epidermidis | B9470 | 4 | (0.14) | 0.12 | (0.004) | 32 |
| | | B9473 | 16 | (0.58) | 4 | (0.14) | 4 |
| 3 | S. lugdunensis | B9510 | 16 | (0.58) | 0.5 | (0.017) | 32 |
| | | B9476 | 4 | (0.14) | 0.5 | (0.017) | 8 |
| 4 | S. haemolyticus | B9511 | 8 | (0.29) | 0.25 | (0.008) | 32 |
| | | B9478 | 128 | (4.64) | 8 | (0.29) | 16 |

Kill Kinetics in Serum:

To evaluate concentration-dependent bactericidal activity of P128 on CoNS cultures in serum, time-kill assays were performed in accordance with the modified CLSI guidelines.

Method:

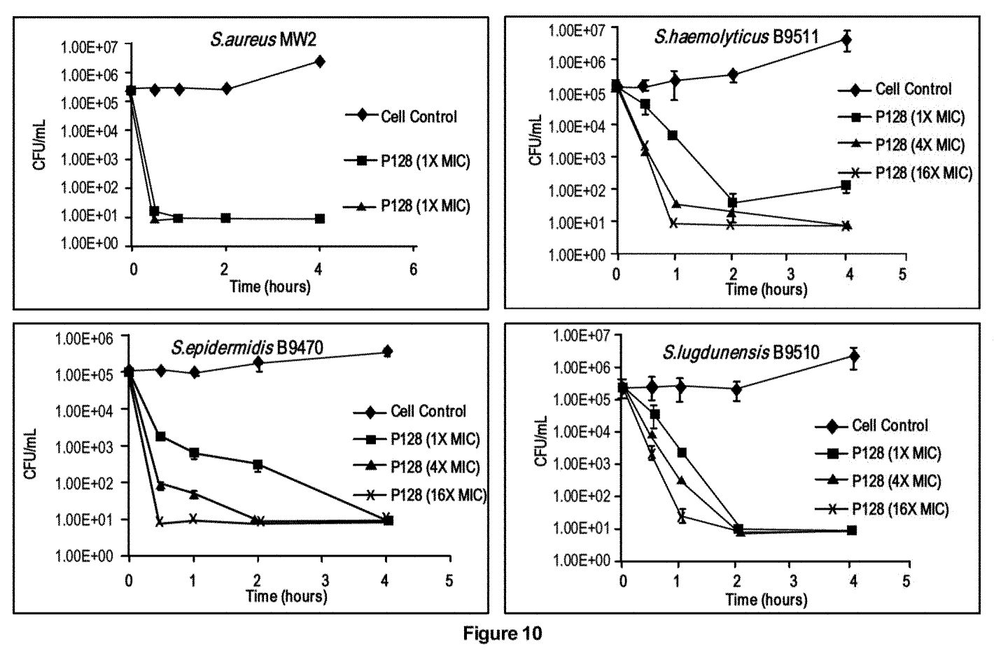

The strains were grown in CAMHB to a density of approximately 1×10⁸ CFU/ml and diluted in CAMHB to obtain 20 ml of 5×10⁵ CFU/ml. This was pelleted and resuspended in fetal calf serum (FCS). A 300 μL aliquot of the cells was sampled for quantification (‘0’ hour reading). From the remaining suspension, 4 samples of 2.7 ml were dispensed in glass vials. One of the vials was left as a control and 300 μL of P128 in serum was added to achieve concentrations corresponding to MIC, 4×MIC, and 16×MIC in the remaining vials. The vials were incubated at 37° C. with shaking at 200 rpm and 300 μL samples were withdrawn at stipulated time points to assess the viability of the cultures. The CFUs in each sample were determined by plating 100 μL of neat and diluted culture suspensions on LB agar plates followed by incubation for 18 h at 37° C. (FIG. 10). The detection limit in time-kill assays was 10 CFU/ml. For serum TKK, the 24 h P128 MIC values on S. epidermidis, S. haemolyticus, and S. lugdunensis were 0.25 μg/ml, 0.25 μg/ml, and 0.50 μg/ml, respectively.

### XX. Efficacy of P128 on In Vivo Biofilm

Observations made in biofilm research have been made mostly in in vitro models. While in vitro biofilm analyses can identify promising anti-biofilm approaches, translation to in vivo situations and on host contribution to the in vivo dynamics of infections on medical devices and indwelling materials would best be validated by in vivo studies. Biofilms represent a niche for microorganisms where they are protected from both the host immune system and typical antimicrobial therapies, features which may lead to significantly enhanced virulence of the bacteria, or causing infections that are difficult to treat without special techniques.

XXI. Evaluation of Effect of P128 and Combinations of P128 with SoC Antibiotics on In Vivo-Biofilms on Catheters

Rat models of central venous catheter associated biofilm infection are reported in literature. See Ebert, et al. (2011) “Development of a rat central venous catheter model for evaluation of vaccines to prevent Staphylococcus epidermidis and Staphylococcus aureus early biofilms” Hum. Vaccines 7:630-638. doi:10.4161/hv.7.6.15407; and Chauhan, et al. (2012) “A Rat Model of Central Venous Catheter to Study Establishment of Long-Term Bacterial Biofilm and Related Acute and Chronic Infections” PLoS ONE 7(5): e37281. doi:10.1371/journal.pone.0037281. A polyurethane catheter is placed into right jugular vein of Wistar albino rats and advanced toward the cranial vena cava. The catheter is held in place by ligating proximally and distally with sterile suture and exteriorized to dorsal surface through a midline scapular incision. Twenty four hours after the catheterization, animals are challenged with S. aureus or S. epidermidis through the tail vein. The lowest level of bacteria that causes an observable biofilm in 72 to 96 hours after challenge is first determined. Subsets of animals are implanted with a catheter in which a biofilm has been allowed to form in vitro. Animals challenged with bacteria or implanted with infected catheters are treated with P128 and combinations of P128 with different Standard of care (SoC) antibiotics (e.g., gentamycin, oxacillin, vancomycin, ciprofloxacin, linezolid, or daptomycin) through suitable parenteral routes. Twenty four hours after the last treatment, blood samples are collected and animals euthanized to surgically remove the catheter for evaluation. The bacterial burden in blood is determined and catheters are evaluated for extent of biofilm formation and bacterial load. Because of rapid and potent antibiofilm activity of P128 coupled with prolonged antibacterial effect of SoC antibiotics, significant reduction in CFUs or complete eradication of biofilms is observed in the case of the combination group, indicating synergy.

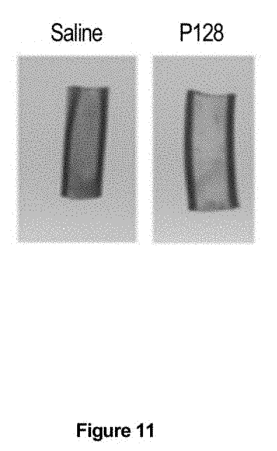

Another in vivo model of biofilm (Kadurugamuwa et al. 2003. “Direct continuous method for monitoring biofilm infection in a mouse model” Infection and immunity, 71(2):882-890) for efficacy evaluation of antibacterial agents involves placement of a catheter in the subcutaneous space and simulating conditions of catheter-associated biofilm formation and infection. P128 was tested in this model. Eight-nine week old female BALB/c mice 25 g were rendered neutropenic with cyclophosphamide and were operated under ketamine-xylazine anesthesia. A 1-cm incision was made in the dorsal neck surface by aseptic technique and subcutaneous pouch was created. Catheter segments of 1 cm length pre-incubated with MRSA MW2 suspension (6×10³ CFUs/mL) for 4 hrs, were placed in the subcutaneous space and the wounds were closed with suture thereafter. As uninfected control, one group of mice were surgically operated and a sterile catheter segment was placed in the cavity. At 24 hours post-catheter implantation, mice were treated with P128 (800 μg per animal, subcutaneously) or with the placebo (saline). Treatments were given thrice a day at 2 hr intervals for three days and 1 hr after the last treatment, the catheters were removed, biofilms were harvested and CFUs recovered were plated on culture media and enumerated. Catheters from a subset of animals were stained with 0.1% safranin, (which stains the bacteria) for 5 min. The CFUs counts obtained are shown in the Table VB1. P128 treatment resulted in greater than 1 log reduction in CFUs compared to the control group. There were no bacteria detected in the catheter recovered from one of the P128-treated animals. Safranin staining also showed eradication of biofilm in catheters from P128 treated animals (FIG. 11).

TABLE VB1
Efficacy of P128 in in vivo biofilm model.
CFUs recovered from catheter biofilms.

| | Animal | CFU | Avg. CFU |
|---|---|---|---|
| Group | No. | (Log10)/ml | Log(10)/ml |
| Saline control; | 1 | 4.54 | 4.5 |
| 3 doses/day for 3 days | 2 | 4.18 | |
| | 3 | 4.28 | |
| | 4 | 5.15 | |
| P128 (800 μg per dose), | 1 | 4.15 | 2.8 |
| 3 doses/day for 3 days | 2 | 3.12 | |
| | 3 | 1.00 | |
| | 4 | Nil | |

For evaluation of synergy with daptomycin, mouse model of biofilm described above was used with few modifications. Eight-nine week old female BALB/c mice 25 g were rendered neutropenic with cyclophosphamide and were operated under ketamine-xylazine anesthesia. A 1-cm incision was made in the dorsal neck surface by aseptic technique and subcutaneous pouch was created. Catheter segments measuring 1 cm were placed in the subcutaneous space, and the wounds closed with suture. At 72 hours post-implantation. MRSA MW2 bacterial inoculum (2.1×10⁷ CFU per animal) was injected subcutaneously onto the catheter. At 24 hrs post-challenge, animals were treated with either P128 (800 μg per animal, subcutaneous), or daptomycin (12.5 mg/kg, subcutaneous) or a combination of both. Daptomycin was given once every day for three days whereas P128 was administered thrice a day at 2 hr interval for three days. 1 hr after the last treatment, animals were euthanized and catheters were collected and recovered CFUs were enumerated. Compared to other treatment groups, no CFUs were obtained in two animals treated with the combination P128 and daptomycin indicating complete eradication of biofilms (Table VB2).

TABLE VB2
Synergy of P128 with vancomycin in in vivo biofilm model.
CFUs recovered from biofilm formed on catheter.

| | Animal | CFU |
|---|---|---|
| Group | No. | (Log10)/ml |
| Saline control | 1 | 4.0 |
| | 2 | 2.8 |
| | 3 | 3.1 |
| Daptomycin (12.5 mg/kg), | 1 | 4.9 |
| 1 dose per day, for 3 days | 2 | 2.6 |
| | 3 | 3.0 |
| | 4 | 2.7 |
| P128 (800 μg per dose), | 1 | 2.5 |
| 3 doses/day for 3 days | 2 | 2.0 |
| | 3 | 2.7 |
| Daptomycin (12.5 mg/kg) + | 1 | 3.5 |
| P128 (800 μg) | 2 | 3.2 |
| | 3 | Nil |
| | 4 | Nil |

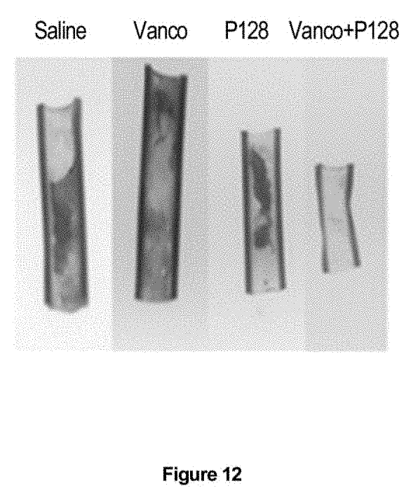

For evaluation of synergy with vancomycin, the mouse model of biofilms described for daptomycin synergy, was used with a few modifications. Eight-nine week old female BALB/c mice, ˜25 g were rendered neutropenic with cyclophosphamide and were operated under ketamine-xylazine anesthesia. A 1-cm incision was made in the dorsal neck surface by aseptic technique and a subcutaneous pouch was created. A 1-cm catheter segment was placed in the subcutaneous space and the wound closed with suture. 24 hours post-implantation, bacterial inoculum (2.5×10⁷ CFU per animal) was injected subcutaneously onto the catheter. 24 hrs post-challenge, animals were treated with either P128 (800 μg per animal per dose, sc), or vancomycin (55 mg/kg, sc), or a combination of both. Three doses of vancomycin were given every 12 hrs whereas P128 was administered thrice a day at 2 hr intervals for two days. Thirty minutes post last-treatment, animals were euthanized and catheters were collected and recovered CFUs were enumerated. Biofilms were also visualized by staining the catheters with 0.1% safranin. Compared to either P128 or vancomycin, there was reduction in CFU counts in two animals and no CFUs were obtained in two animals indicating complete eradication (Table VB3). Safranin staining also indicated complete eradication of biofilm in combination group compared to either drug alone (FIG. 12).

TABLE VB3
Synergy of P128 with vancomycin in in vivo biofilm model.
CFUs recovered from biofilm formed on catheter.

| | Animal | CFU | Avg. CFU |
|---|---|---|---|
| Groups | No. | (Log10)/ml | (Log10)/ml |
| Saline | 1 | 4.5 | 4.9 |
| | 2 | 5.6 | |
| | 3 | 6.1 | |
| | 4 | 3.3 | |
| Vancomycin (55 mg/kg), three | 1 | 5.7 | 4.9 |
| doses at 12 h interval | 2 | 3.5 | |
| | 3 | 5.5 | |
| | 4 | 5.0 | |
| | 5 | 5.0 | |
| P128 (800 μg per dose), 3 | 1 | 3.4 | 3.8 |
| doses/day for 2 days | 2 | 3.7 | |
| | 3 | 3.5 | |
| | 4 | 4.5 | |
| Vancomycin (55 mg/kg) + | 1 | Nil | 2.2 |
| P128 (800 ug per animal) | 2 | 2.3 | |
| | 3 | 2.1 | |
| | 4 | Nil | |

XXII. Synergy of P128 with SoC Antibiotics in Other In Vivo Catheter Biofilm Models

Catheter based biofilm models have been described in the literature. Zhu, et al. (2007). Staphylococcus aureus Biofilm Metabolism and the Influence of Arginine on Polysaccharide Intercellular Adhesin Synthesis, Biofilm Formation, and Pathogenesis. Infect Immun. 75(9): 4219-4226. This in vivo catheter model in mice is used with few modifications. Mice are rendered neutropenic with cyclophosphamide and operated under ketamine and xylazine anaesthesia. 1 cm catheter segments are placed either in subcutaneous or intraperitoneal or intramuscular space. 24 hrs following implantation, bacterial inoculum (MRSA or S. epidermidis) is injected into the respective compartments. In another set of animals catheters are pre-incubated in bacterial cultures so that a biofilm is preformed and then implanted into subcutaneous or intraperitoneal or intramuscular space. In a third set of animals, a catheter is precolonised with bacterial inoculum and then implanted. At different time points following injection of bacterial inoculum or implantation of catheter, animals are treated with P128 by either subcutaneous or intraperitoneal or intravenous or intramuscular route. To evaluate the drug synergy one set of animals are treated with standard of care antibiotics (e.g., gentamycin, oxacillin, vancomycin, ciprofloxacin, linezolid, or daptomycin) along with P128. Animals are sacrificed at appropriate time after the treatment and catheters are removed. To quantify the biofilms, catheters are washed gently with PBS to remove planktonic cells, then the adherent biofilm is scraped with an inoculation loop and recovered bacteria are plated to enumerate CFUs. Because of rapid and potent bactericidal effect of P128 coupled with prolonged effect of SoC antibiotics, a significant reduction in CFUs or eradication of biofilm is observed in animals treated with combination of P128 and antibiotic compared to either drug alone.

XXI. Synergy of P128 with SoC Antibiotics in Infective Endocarditis

A rat model of infective endocarditis is used. Fernandez, et al. (2012). Synergistic activity of ceftobiprole and vancomycin in a rat model of infective endocarditis caused by methicillin-resistant and glycopeptide-intermediate Staphylococcus aureus. Antimicrob Agents Chemother. 56(3):1476-84. Female Wistar albino rats are operated under ketamine and xylazine anaesthesia Right carotid artery is cannulated and polyethylene catheter is placed near the atrio-ventricular valve. Two days later, animals are challenged with MRSA MW2 through tail vein. Twenty four hours post-challenge, animals are treated with appropriate doses of P128 (IV bolus or infusion) or standard of care antibiotics (e.g., vancomycin, daptomycin, linezolid, oxacillin, etc.) or both. Animals are euthanized at appropriate time after the last dosing, vegetations are collected and bacterial load is determined. Significantly higher CFU reduction is observed in vegetations of animals treated with P128, and with combination of P128 and antibiotic.

XXIV. Evaluation of Synergistic Effect of P128 with Antibiotics in Bacteremia

A standard mouse model of S. aureus bacteremia was used. Zuluaga, et al. (2006) “Neutropenia induced in out bred mice by a simplified low-dose cyclophosphamide regimen: Characterization and applicability to diverse experimental models of infectious diseases” BMC Infect. Dis. 6:55-64. Female BALB/c mice of 8-9 weeks age were challenged by intraperitoneal route with S. aureus COL at 10⁹ CFU per animal. After the bacterial challenge, animals were treated through parenteral routes with P128 alone or P128 in combination with standard of care antibiotics (e.g., vancomycin, daptomycin, linezolid, oxacillin, etc.). Animals were monitored for survival for a period of 72 hours. In this model of infection, normal as well as immunocompromised (neutropenia induced by cyclophosphamide) mice were used to evaluate P128 and antibiotic-P128 combinations.

A. In Vivo Efficacy of P128 in Combination with Vancomycin:

Eight to nine weeks old female BALB/c mice were challenged intraperitoneally with 10⁹ CFU of S. aureus COL. At 2 hours post-infection, animals were treated with either P128 at 0.2 mg/kg via intraperitoneal route, or vancomycin at 55 mg/kg by subcutaneous route, or a combination of both by respective routes. Groups treated with only vancomycin or combination of P128 and vancomycin were administered a second dose of vancomycin 12 hours after the first dose. At 72 hours post-infection, untreated animals did not survive in this model of S. aureus infection. As expected, in the untreated control group >85% of animals succumbed to the infection. P128 and vancomycin yielded 30 and 46% survival, respectively by themselves. Treatment with both P128 and vancomycin as a combination resulted in 84% survival demonstrating that P128 and vancomycin combination yields a vastly superior efficacy in comparison to either drug alone (Table 14).

TABLE 14
Efficacy of P128, vancomycin and combination in
rescuing animals from lethal S. aureus infection
in terms of survival at 72 h post infection

| Group | | |
|---|---|---|
| (26 mice per group) | Dose | Survival (%) |
| Infection control | — | 12 |
| P128 | 0.2 mg/kg; single dose | 30 |
| Vancomycin | 55 mg/kg; 2 doses | 46 |
| P128 + Vancomycin | P128-0.2 mg/kg; single dose + | 84 |
| | Vancomycin-55 mg/kg; 2 doses | |

B. In Vivo Efficacy of P128 in Combination with Daptomycin:

In the same model, efficacy of daptomycin in combination with P128 was evaluated. At 2 hours post-infection, animals were treated with either P128 at 0.2 mg/kg administered by intraperitoneal route, or daptomycin at 12.5 mg/kg administered by subcutaneous route; or a combination of both administered by respective routes. Groups treated with only daptomycin or combination of P128 and daptomycin were administered a second dose of daptomycin 12 hours after the first daptomycin dose. At 72 hours post-infection, P128 and daptomycin by themselves resulted in 45% survival, whereas treatment with both P128 and daptomycin as a combination resulted in 88% survival of animals, demonstrating that P128 works effectively at very low concentrations, in combination with daptomycin, and that the combination is superior to either drug alone (see Table 15).

TABLE 15
Efficacy of P128, daptomycin and combination in
rescuing animals from lethal S. aureus infection
in terms of survival at 72 h post infection

| Group | | |
|---|---|---|
| (24 mice per group) | Dose | Survival (%) |
| Infection control | — | 12 |
| P128 | 0.2 mg/kg; single dose | 45 |
| Daptomycin | 12.5 mg/kg; 2 doses | 45 |
| P128 + daptomycin | P128-0.2 mg/kg; single dose + | 88 |
| | Daptomycin-12.5 mg/kg | |

C. Efficacy of P128 and Combination of P128 and Vancomycin in a Mouse Model of S. aureus with Mucin-Enhanced Virulence:

Eight to nine weeks old female BALB/c mice were challenged intraperitoneally with 10⁹ CFU of S. aureus USA300. At 2 hours post-infection, animals were treated with either P128 at 2.5 mg/kg intraperitoneally, or vancomycin at 27.5 mg/kg subcutaneously, or combination of both. Groups treated with only vancomycin or combination of P128 and vancomycin were administered with a second dose of vancomycin at 12 hours after the first treatment. At 72 hours post-infection, P128 and vancomycin yielded 56 and 44% survival, respectively. Treatment with both P128 and vancomycin resulted in 81% survival demonstrating that P128 and vancomycin combination yields superior efficacy in comparison to either drug alone (see Table 16).

TABLE 16
Efficacy of P128, vancomycin and combination in rescuing
animals from mucin-enhanced lethal S. aureus infection
in terms of survival at 72 h post infection

| Group | Dose | Survival (%) |
|---|---|---|
| Infection control | — | 12 |
| P128 | 2.5 mg/kg | 56 |
| Vancomycin | 27.5 mg/kg | 43 |
| P128 + vancomycin | 2.5 mg/kg + 27.5 mg/kg | 81 |

It is understood that the examples and embodiments described herein are for illustrative purposes only and that various modifications or changes in light thereof will be suggested to persons skilled in the art and are to be included within the spirit and purview of this application and scope of the appended claims. All publications, accession numbers, patents, and patent applications cited herein are hereby incorporated by reference in their entirety for all purposes.

INFORMAL SEQUENCE LISTING

```
SEQ ID NO: 1
Met Ser Leu Asp Ser Leu Lys Lys Tyr Asn Gly Lys Leu Pro Lys His
    1 5 10 15
Asp Pro Ser Phe Val Gln Pro Gly Asn Arg His Tyr Lys Tyr Gln Lys
                          20 25 30
Thr Trp Tyr Ala Tyr Asn Arg Arg Gly Gln Leu Gly Ile Pro Val Pro
                  35 40 45
Leu Trp Gly Asp Ala Ala Asp Trp Ile Gly Gly Ala Lys Gly Ala Gly
          50 55 60
Tyr Gly Val Gly Arg Thr Pro Lys Gln Gly Ala Lys Val Ile Trp Gln
  65 70 75 80
Arg Gly Val Gln Gly Gly Ser Pro Gln Tyr Gly His Val Ala Phe Val
                                  85 90 95
Glu Lys Val Leu Asp Gly Gly Lys Lys Ile Phe Ile Ser Glu His Asn
                        100 105 110
Tyr Ala Thr Pro Asn Gly Tyr Gly Thr Arg Thr Ile Asp Met Ser Ser
                115 120 125
Ala Ile Gly Lys Asn Ala Gln Phe Ile Tyr Asp Lys Lys Leu Glu Thr
        130 135 140
Pro Asn Thr Gly Trp Lys Thr Asn Lys Tyr Gly Thr Leu Tyr Lys Ser
145 150 155 160
Glu Ser Ala Ser Phe Thr Pro Asn Thr Asp Ile Ile Thr Arg Thr Thr
                                165 170 175
Gly Pro Phe Arg Ser Met Pro Gln Ser Gly Val Leu Lys Ala Gly Gln
                        180 185 190
Thr Ile His Tyr Asp Glu Val Met Lys Gln Asp Gly His Val Trp Val
                195 200 205
Gly Tyr Thr Gly Asn Ser Gly Gln Arg Ile Tyr Leu Pro Val Arg Thr
        210 215 220
Trp Asn Lys Ser Thr Asn Thr Leu Gly Val Leu Trp Gly Thr Ile Lys
225 230 235 240
```

What is claimed is:

1. A method comprising administering a P128 chimera to a subject, wherein said administering prevents formation of, or destroys, a biofilm comprising Staphylococcus in said subject.

2. The method of claim 1, wherein said administering prevents formation of said biofilm.

3. The method of claim 1, wherein said administering destroys said biofilm.

4. The method of claim 1, wherein said biofilm forms on a catheter, implant, prosthesis, valve, surface, bandage, or foreign body.

5. A method comprising administering to a subject a synergistic therapy or combination composition comprising:
(a) a P128 chimera; and
(b) an antibiotic selected from oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, and azithromycin;
wherein said combination prevents formation of, or destroys, a biofilm comprising Staphylococcus in said subject.

6. The method of claim 5, wherein said antibiotic is:
(a) oxacillin
(b) gentamycin;
(c) vancomycin;
(d) ciprofloxacin;
(e) linezolid;
(f) daptomycin;
(g) cefazolin;
(h) clindamycin;
(i) rifampicin;
(j) tigecycline;
(k) dalbavancin;
(l) telavancin;
(m) ceftobiprole;
(n) co-trimethaxazole; or
(o) azithromycin.

7. The method of claim 5, wherein said combination prevents formation of said biofilm.

8. The method of claim 5, wherein said combination destroys said biofilm.

9. The method of claim 5, wherein said biofilm forms on a catheter, implant, prosthesis, valve, surface, bandage, or foreign body.

10. A method comprising administering a synergistic therapy or combination composition comprising:
(a) a P128 chimera; and
(b) an antibiotic selected from oxacillin, linezolid, daptomycin, gentamycin, vancomycin, ciprofloxacin, cefazolin, clindamycin, rifampicin, tigecycline, dalbavancin, telavancin, ceftobiprole, co-trimethaxazole, and azithromycin;
wherein said synergistic combination prevents formation of, or destroys, a biofilm comprising Staphylococcus.

11. The method of claim 10, wherein said antibiotic is:
(a) oxacillin;
(b) linezolid;
(c) daptomycin;
(d) gentamycin;
(e) vancomycin;
(f) ciprofloxacin;
(g) cefazolin;
(h) clindamycin;
(i) rifampicin;
(j) tigecycline;
(k) dalbavancin;
(l) telavancin,
(m) ceftobiprole;
(n) co-trimethaxazole; or
(o) azithromycin.

12. The method of claim 10, wherein said administering prevents formation of said biofilm.

13. The method of claim 10, wherein said administering destroys said biofilm.

14. The method of claim 10, wherein said biofilm forms on a catheter, implant, prosthesis, valve, surface, bandage, or foreign body.

15. The method of claim 10, wherein said biofilm is in vitro.

16. A method comprising administering a synergistic therapy or combination compound comprising:
(a) a P128 chimera; and
(b) an antibiotic selected from oxacillin, vancomycin, linezolid, daptomycin, gentamycin, ciprofloxacin, cefazolin, clindamycin, rifampicin, tigecycline, dalbavancin, telavancin, ceftobiprole, co-trimethaxazole, and azithromycin;
wherein said synergistic combination reduces growth of planktonic Staphylococcus cells.

17. The method of claim 16, wherein said antibiotic is:
(a) oxacillin;
(b) vancomycin;
(c) linezolid;
(d) daptomycin;
(e) gentamycin;
(f) ciprofloxacin;
(g) cefazolin;
(h) clindamycin;
(i) rifampicin;
(j) tigecycline;
(k) dalbavancin;
(l) telavancin;
(m) ceftobiprole;
(n) co-trimethaxazole; or
(o) azithromycin.

18. The method of claim 16, wherein said reduction in growth is:
(a) at least about 10-40%;
(b) at least about 40-80%; or
(c) at least about 80-99% or more.

19. The method of claim 16, wherein said reduction in growth reduces said cells over a period of said administering.

20. A method comprising administering to a subject a combination therapy comprising administering components:
(a) a P128 chimera; and
(b) an antibiotic selected from oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, and azithromycin;
wherein said combination therapy reduces a target infection where said infection is resistant to either component alone.

21. A method comprising administering to a subject a combination therapy comprising administering:
(a) a P128 chimera; and
(b) an antibiotic selected from oxacillin, gentamycin, vancomycin, ciprofloxacin, linezolid, daptomycin, cefazolin, clindamycin, rifampicin, tigecycline, dalbavancin, telavancin, ceftobiprole, co-trimethaxazole, and azithromycin;
wherein said combination therapy reduces the population or growth of small colony variants of a target infection.
